(19) United States
(12) Patent Application Publication
(10) Pub. No.: US 2024/0150760 A1
(43) Pub. Date: May 9, 2024

(54) ANTISENSE OLIGOMERS FOR TREATMENT OF CONDITIONS AND DISEASES

(71) Applicant: Stoke Therapeutics, Inc., Bedford, MA (US)

(72) Inventors: Isabel AZNAREZ, Jamaica Plain, MA (US); Zhou HAN, Natick, MA (US)

(21) Appl. No.: 18/512,785

(22) Filed: Nov. 17, 2023

Related U.S. Application Data
(62) Division of application No. 17/127,581, filed on Dec. 18, 2020, now Pat. No. 11,873,490.
Application No. 17/127,581 is a continuation of application No. 16/561,952, filed on Sep. 5, 2019, now Pat. No. 10,913,947.
Application No. 16/561,952 is a continuation of application No. PCT/US2018/048031, filed on Aug. 24, 2018.
(60) Provisional application No. 62/550,462, filed on Aug. 25, 2017.
(60) Provisional application No. 62/575,901, filed on Oct. 23, 2017.
(60) Provisional application No. 62/667,356, filed on May 4, 2018.
(60) Provisional application No. 62/671,745, filed on May 15, 2018.

(51) Int. Cl. C12N 15/113; A61K 9/00; A61P 25/00; A61P 25/12

(52) U.S. Cl. CPC C12N 15/113; A61K 9/0019; A61K 9/0085; A61P 25/00; A61P 25/12; C12N 15/1138; C12N 2310/11; C12N 2310/321; C12N 2320/32; C12N 2320/35

(57) ABSTRACT

Alternative splicing events in SCN1A gene can lead to non-productive mRNA transcripts which in turn can lead to aberrant protein expression, and therapeutic agents which can target the alternative splicing events in SCN1A gene can modulate the expression level of functional proteins in Dravet Syndrome patients and/or inhibit aberrant protein expression. Such therapeutic agents can be used to treat a condition caused by SCN1A, SCN8A or SCN5A protein deficiency.

### CROSS-REFERENCE

This application is a divisional of U.S. patent application Ser. No. 17/127,581, filed Dec. 18, 2020, which is a continuation of U.S. patent application Ser. No. 16/561,952, filed on Sep. 5, 2019, now issued as U.S. Pat. No. 10,913,947, which is a continuation of international patent application no. PCT/US2018/048031, filed on Aug. 24, 2018 which claims the benefit of U.S. Provisional Application No. 62/550,462, filed on Aug. 25, 2017, U.S. Provisional Application No. 62/575,901, filed on Oct. 23, 2017, U.S. Provisional Application No. 62/667,356, filed on May 4, 2018, and U.S. Provisional Application No. 62/671,745, filed on May 15, 2018, each of which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 14, 2023, is named 47991_719_401_SL.xml and is 1,189,844 bytes in size.

### BACKGROUND OF THE INVENTION

Nervous system disorders are often associated with channelopathy, characterized by the disturbed function of ion channels that mediate neuronal excitability, neuronal interactions, and brain functions at large. Mutations in the SCN1A gene, which is part of the SCN1A-SCN2A-SCN3A gene cluster that encodes alpha-pore forming subunits of the neuronal voltage gated sodium channel, are associated with development of disease number of diseases and conditions, such as Dravet Syndrome (DS) (Miller, et al., 1993-2015, GeneReviews, Eds. Pagon R A, et al. Seattle (WA): University of Washington, Seattle, Bookshelf ID: NBK1318, and Mulley, et al., 2005, Hum. Mutat. 25: 535-542).

### SUMMARY OF THE INVENTION

Disclosed herein, in certain embodiments, is a method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell. In some embodiments, the therapeutic agent (a) binds to a targeted portion of the NMD exon mRNA encoding SCN1A; (b) modulates binding of a factor involved in splicing of the NMD exon mRNA; or (c) a combination of (a) and (b). In some embodiments, the therapeutic agent interferes with binding of the factor involved in splicing of the NMD exon from a region of the targeted portion. In some embodiments, the targeted portion is proximal to the NMD exon. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon. In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon. In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon. In some embodiments, the targeted portion is located in an intronic region between two canonical exonic regions of the NMD exon mRNA encoding SCN1A, and wherein the intronic region contains the NMD exon. In some embodiments, the targeted portion at least partially overlaps with the NMD exon. In some embodiments, the targeted portion at least partially overlaps with an intron upstream of the NMD exon. In some embodiments, the targeted portion comprises 5′ NMD exon-intron junction or 3′ NMD exon-intron junction. In some embodiments, the targeted portion is within the NMD exon. In some embodiments, the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, the NMD exon mRNA encoding SCN1A comprises a sequence with at least about 80%, 85%, 90%, 95%, 97%, or 100% sequence identity to any one of SEQ ID NOs: 2 or 7-10. In some embodiments, the NMD exon mRNA encoding SCN1A is encoded by a genetic sequence with at least about 80%, 85%, 90%, 95%, 97%, or 100% sequence identity to SEQ ID NOs: 1 or 3-6. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh37/hg19: chr2:166,863,803.

In some embodiments, the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of genomic site GRCh37/hg19: chr2:166,863,803. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh37/hg19: chr2:166,863,740. In some embodiments, the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of genomic site GRCh37/hg19: chr2:166,863,740. In some embodiments, the targeted portion of the NMD exon mRNA encoding SCN1A comprises a sequence with at least 80%, 85%, 90%, 95%, 97%, or 100% sequence identity to a region comprising at least 8 contiguous nucleic acids of SEQ ID NO: SEQ ID NOs: 2 or 7-10. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 21-67, 210-256, or 304-379. In some embodiments, the targeted portion of the NMD exon mRNA encoding SCN1A is within the non-sense mediated RNA decay-inducing exon 20× of SCN1A. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 42-50, or 231-239. In some embodiments, the targeted portion of the NMD exon mRNA encoding SCN1A is upstream or downstream of the non-sense mediated RNA decay-inducing exon 20× of SCN1A.

In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 21-38, 53-67, 210-227, or 242-256. In some embodiments, the targeted portion of the NMD exon mRNA comprises an exon-intron junction of exon 20× of SCN1A. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 39-41, 51, 52, 228-230, 240, or 241. In some embodiments, the therapeutic agent promotes exclusion of the NMD exon from the processed mRNA encoding SCN1A protein. In some embodiments, exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent increases level of the processed mRNA encoding SCN1A protein in the cell. In some embodiments, an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell. In some embodiments, the therapeutic agent increases expression of SCN1A protein in the cell. In some embodiments, an amount of SCN1A produced in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell. In some embodiments, the therapeutic agent inhibits exclusion of the NMD exon from the processed mRNA encoding SCN1A protein. In some embodiments, exclusion of the NMD exon from the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent decreases level of the processed mRNA encoding SCN1A protein in the cell. In some embodiments, an amount of the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell. In some embodiments, the therapeutic agent decreases expression of SCN1A protein in the cell. In some embodiments, an amount of SCN1A produced in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell. In some embodiments, the therapeutic agent is an antisense oligomer (ASO) and wherein the antisense oligomer comprises a backbone modification comprising a phosphorothioate linkage or a phosphorodiamidate linkage. In some embodiments, the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety. In some embodiments, the therapeutic agent is an antisense oligomer (ASO) and wherein the antisense oligomer comprises at least one modified sugar moiety. In some embodiments, each sugar moiety is a modified sugar moiety. In some embodiments, the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. In some embodiments, the therapeutic agent is an antisense oligomer (ASO) and wherein the antisense oligomer is at least 80%, at least 85%, at least 90%, at least 95%, at least 98%, at least 99%, or 100%, complementary to the targeted portion of the NMD exon mRNA encoding the protein. In some embodiments, the method further comprises assessing SCN1A mRNA or protein expression. In some embodiments, the cells are ex vivo.

Disclosed herein, in certain embodiments, is a method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject. In some embodiments, the therapeutic agent (a) binds to a targeted portion of the NMD exon mRNA encoding SCN1A; (b) modulates binding of a factor involved in splicing of the NMD exon mRNA; or (c) a combination of (a) and (b). In some embodiments, the therapeutic agent interferes with binding of the factor involved in splicing of the NMD exon from a region of the targeted portion. In some embodiments, the targeted portion is proximal to the NMD exon. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon. In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon. In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon. In some embodiments, the targeted portion is located in an intronic region between two canonical exonic regions of the NMD exon mRNA encoding SCN1A, and wherein the intronic region contains the NMD exon. In some embodiments, the targeted portion at least partially overlaps with the NMD exon. In some embodiments, the targeted portion at least partially overlaps with an intron upstream of the NMD exon. In some embodiments, the targeted portion comprises 5′ NMD exon-intron junction or 3′ NMD exon-intron junction. In some embodiments, the targeted portion is within the NMD exon.

In some embodiments, the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, the NMD exon mRNA encoding SCN1A comprises a sequence with at least about 80%, 85%, 90%, 95%, 97%, or 100% sequence identity to any one of SEQ ID NOs: 2 or 7-10. In some embodiments, the NMD exon mRNA encoding SCN1A is encoded by a genetic sequence with at least about 80%, 85%, 90%, 95%, 97%, or 100% sequence identity to SEQ ID NOs: 1 or 3-6. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh37/hg19: chr2:166,863,803. In some embodiments, the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of genomic site GRCh37/hg19: chr2:166,863,803. In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh37/hg19: chr2:166,863,740. In some embodiments, the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of genomic site GRCh37/hg19: chr2:166,863,740. In some embodiments, the targeted portion of the NMD exon mRNA encoding SCN1A comprises a sequence with at least 80%, 85%, 90%, 95%, 97%, or 100% sequence identity to a region comprising at least 8 contiguous nucleic acids of SEQ ID NO: SEQ ID NOs: 2 or 7-10. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 21-67, 210-256, or 304-379. In some embodiments, the targeted portion of the NMD exon mRNA encoding SCN1A is within the non-sense mediated RNA decay-inducing exon 20× of SCN1A. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 42-50, or 231-239. In some embodiments, the targeted portion of the NMD exon mRNA encoding SCN1A is upstream or downstream of the non-sense mediated RNA decay-inducing exon 20× of SCN1A.

In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 21-38, 53-67, 210-227, or 242-256. In some embodiments, the targeted portion of the NMD exon mRNA comprises an exon-intron junction of exon 20× of SCN1A. In some embodiments, the therapeutic agent is an antisense oligomer (ASO) and wherein the ASO comprises a sequence that is at least about 80%, 85%, 90%, 95%, 97%, or 100% identity to any one of SEQ ID NOs: 39-41, 51, 52, 228-230, 240, or 241. In some embodiments, the therapeutic agent promotes exclusion of the NMD exon from the processed mRNA encoding SCN1A protein. In some embodiments, exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent increases level of the processed mRNA encoding SCN1A protein in the cell. In some embodiments, an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell. In some embodiments, the therapeutic agent increases expression of SCN1A protein in the cell. In some embodiments, an amount of SCN1A produced in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell. In some embodiments, the therapeutic agent inhibits exclusion of the NMD exon from the processed mRNA encoding SCN1A protein. In some embodiments, exclusion of the NMD exon from the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent decreases level of the processed mRNA encoding SCN1A protein in the cell. In some embodiments, an amount of the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell. In some embodiments, the therapeutic agent decreases expression of SCN1A protein in the cell. In some embodiments, an amount of SCN1A produced in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell. In some embodiments, the therapeutic agent is an antisense oligomer (ASO) and wherein the antisense oligomer comprises a backbone modification comprising a phosphorothioate linkage or a phosphorodiamidate linkage. In some embodiments, the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety. In some embodiments, the therapeutic agent is an antisense oligomer (ASO) and wherein the antisense oligomer comprises at least one modified sugar moiety. In some embodiments, each sugar moiety is a modified sugar moiety. In some embodiments, the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. In some embodiments, the therapeutic agent is an antisense oligomer (ASO) and wherein the antisense oligomer is at least 80%, at least 85%, at least 90%, at least 95%, at least 98%, at least 99%, or 100%, complementary to the targeted portion of the NMD exon mRNA encoding the protein. In some embodiments, the method further comprises assessing SCN1A mRNA or protein expression. In some embodiments, the disease or condition is induced by a loss-of-function mutation in Nay1.1.

In some embodiments, the disease or condition is associated with haploinsufficiency of the SCN1A gene, and wherein the subject has a first allele encoding a functional SCN1A, and a second allele from which SCN1A is not produced or produced at a reduced level, or a second allele encoding a nonfunctional SCN1A or a partially functional SCN1A. In some embodiments, the disease or condition is encephalopathy. In some embodiments, the encephalopathy is epileptic encephalopathy. In some embodiments, the disease or condition is Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or malignant migrating partial seizures of infancy. In some embodiments, GEFS+ is epilepsy, generalized, with febrile seizures plus, type 2. In some embodiments, the Febrile seizure is Febrile seizures, familial, 3A. In some embodiments, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC). In some embodiments, the therapeutic agent promotes exclusion of the NMD exon from the processed mRNA encoding SCN1A protein and increases the expression of SCN1A in the cell. In some embodiments, the therapeutic agent is an antisense oligomer (ASO) and wherein the ASO comprises a sequence that is at least about 80%, 85%, 90%, 95%, 97%, or 100% complimentary to any one of SEQ ID NOs: 22-24, 26, 27, 29-35, 37-62, 64-67, or 304-379. In some embodiments, the disease or condition is induced by a gain-of-function mutation in Nay1.1. In some embodiments, the subject has an allele from which SCN1A is produced at an increased level, or an allele encoding a mutant SCN1A that induces increased activity of Nay1.1 in the cell. In some embodiments, the disease or condition is migraine. In some embodiments, the migraine is migraine, familial hemiplegic, 3. In some embodiments, the disease or condition is a Nay1.1 genetic epilepsy. In some embodiments, the therapeutic agent inhibits exclusion of the NMD exon from the processed mRNA encoding SCN1A protein and decreases the expression of SCN1A in the cell. In some embodiments, the therapeutic agent is an antisense oligomer (ASO) and wherein the ASO comprises a sequence that is at least about 80%, 85%, 90%, 95%, 97%, or 100% complimentary to any one of SEQ ID NOs: 21, 25, 28, 36, or 63. In some embodiments, the subject is a human. In some embodiments, the subject is a non-human animal. In some embodiments, the subject is a fetus, an embryo, or a child. In some embodiments, the therapeutic agent is administered by intrathecal injection, intracerebroventricular injection, intraperitoneal injection, intramuscular injection, subcutaneous injection, intravitreal, or intravenous injection of the subject. In some embodiments, the method further comprises administering a second therapeutic agent to the subject. In some embodiments, the second therapeutic agent is a small molecule. In some embodiments, the second therapeutic agent is an ASO. In some embodiments, the ASO comprises a sequence that is at least about 80%, 85%, 90%, 95%, 97%, or 100% complimentary to any one of SEQ ID NOs: 115-161. In some embodiments, the second therapeutic agent corrects intron retention. In some embodiments, the disease or condition is Alzheimer's Disease, SCN2A encephalopathy, SCN8A encephalopathy, or SCN5A arrhythmia. In some embodiments, the disease or condition is Alzheimer's Disease, SCN2A encephalopathy, SCN8A encephalopathy, or SCN5A arrythmia. In some embodiments, the cells are ex vivo.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C depict a schematic representation of a target mRNA that contains a non-sense mediated RNA decay-inducing exon (NMD exon mRNA) and therapeutic agent-mediated exclusion of the nonsense-mediated mRNA decay-inducing exon to increase expression of the full-length target protein or functional RNA. FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in mRNA, which is in turn translated into higher levels of target protein. FIG. 1C is a schematic representation of therapeutic ASO-mediated exclusion of a nonsense-mediated mRNA decay-inducing exon, which turns a non-productive mRNA into a productive mRNA and increases expression of the full-length target protein from the productive mRNA.

FIG. 2 depicts identification of an exemplary nonsense-mediated mRNA decay (NMD)-inducing exon in the SCN1A gene. The identification of the NMD-inducing exon in the SCN1A gene using comparative genomics is shown, visualized in the UCSC genome browser. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence) (SEQ ID NO: 380). Inclusion of this exon leads to a frameshift and the introduction of a premature termination codon in exon 21 rendering the transcript a target of NMD.

FIG. 3A depicts confirmation of NMD-inducing exon via cycloheximide treatment. RT-PCR analysis using cytoplasmic RNA from DMSO-treated (CHX−) or cycloheximide-treated (CHX+) Neuro 2A (mouse neural progenitor cells) and primers in exon 21 and a downstream exon confirmed the presence of a band corresponding to the NMD-inducing exon (21×). The identity of the product was confirmed by sequencing. Densitometry analysis of the bands was performed to calculate percent exon 21× inclusion of total SCN1A transcript. Treatment of Neuro 2A with cycloheximide (CHX+) to inhibit NMD led to a 2-fold increase of the product corresponding to the NMD-inducing exon 21× in the cytoplasmic fraction (cf. light grey bar, CHX−, to dark grey bar, CHX+).

FIG. 3B depicts confirmation of NMD-inducing exon via cycloheximide treatment. RT-PCR analysis using cytoplasmic RNA from DMSO-treated (CHX−) or cycloheximide-treated (CHX+) RenCell VM (human neural progenitor cells) and primers in exon 20 and exon 23 confirmed the presence of a band corresponding to the NMD-inducing exon (20×). The identity of the product was confirmed by sequencing. Densitometry analysis of the bands was performed to calculate percent exon 20× inclusion of total SCN1A transcript. Treatment of RenCell VM with cycloheximide (CHX+) to inhibit NMD led to a 2-fold increase of the product corresponding to the NMD-inducing exon 20× in the cytoplasmic fraction (cf. light grey bar, CHX−, to dark grey bar, CHX+).

FIG. 4 depicts an exemplary SCN1A exon 20× region ASO walk (SEQ ID NOS 381-383, respectively, in order of appearance). A graphic representation of an ASO walk performed for SCN1A exon 20× region targeting sequences upstream of the 3′ splice site, across the 3′splice site, exon 20×, across the 5′ splice site, and downstream of the 5′ splice site using 2′-MOE ASOs, PS backbone, is shown. ASOs were designed to cover these regions by shifting 5 nucleotides at a time.

FIG. 5A depicts SCN1A exon 20× region ASO walk evaluated by RT-PCR. A representative PAGE shows SYBR-safe-stained RT-PCR products of SCN1A mock-treated (Sham), SMN-control ASO treated (SMN), or treated with a 2′-MOE ASO targeting the exon 20× region as described herein in the Examples and in the description of FIG. 4, at 20 μM concentration in RenCell VM cells by gymnotic uptake. Two products corresponding to exon 20× inclusion (top band) and full-length (exon 20× exclusion, bottom band) were quantified.

FIG. 5B depicts a graph plotting the percent exon 20× inclusion from the data in FIG. 5A. The black line indicates no change with respect to Sham.

FIG. 5C depicts a graph of the full-length products normalized to RPL32 internal control and the fold-change relative to Sham is plotted. The black line indicates a ratio of 1 and no change with respect to Sham.

FIG. 6 depicts an exemplary SCN1A exon 20× region ASO walk evaluated by RT-qPCR. SYBR-green RT-qPCR SCN1A amplification results normalized to RPL32, obtained using the same ASO uptake experiment that were evaluated by SYBR-safe RT-PCR as shown in FIG. 5, are plotted as fold change relative to Sham confirming the SYBR-safe RT-PCR results. The black line indicates a ratio of 1 (no change with respect to sham).

FIG. 7A depicts a table with members of the sodium voltage-gaited channel alpha subunit members. Arrows correspond to bar colors in FIG. 7B. X denotes no expression detected.

FIG. 7B depicts selected ASOs evaluated by Tagman qPCR of SCN1A, SCN2A, SCN3A, SCN8A, and SCN9A to assess target selectivity. Tagman-qPCR amplification results normalized to RPL32, obtained using Ex20×+1, IVS20×+18, and IVS20×+33 ASOs, are plotted as fold change relative to Sham. The black line indicates a ratio of 1 (no change with respect to sham).

FIG. 8A depicts exemplary dose-dependent effect of selected ASO in CXH-treated cells. A representative PAGE showing SYBR-safe-stained RT-PCR products of mouse Scn1a mock-treated (Sham, RNAiMAX alone), or treated with Ex21×+1 2′-MOE ASO targeting the exon 21× (mouse nomenclature, corresponds to human exon 20×), at 30 nM, 80 nM, and 200 nM concentrations in Neuro 2A (mouse neuroblastoma) cells by RNAiMAX transfection is shown.

Ex21×+1 (mouse nomenclature) and Ex20×+1 (human nomenclature) are identical. Two products corresponding to exon 20× inclusion (top band) and full-length (exon 20× exclusion, bottom band) were quantified.

FIG. 8B depicts a graph plotting the percent exon 20× inclusion from the data in FIG. 7A. The black line indicates no change with respect to Sham.

FIG. 8C depicts an exemplary graph of the full-length products normalized to Hprt internal control and fold-change relative to Sham are plotted. The black line indicates a ratio of 1 and no change with respect to Sham.

FIG. 9A depicts exemplary results from intravitreal (IVT) injection of selected ASOs in C57BL6J mice (male, 3 months old). PAGE gels of SYBR-safe-stained RT-PCR products of mouse Scn1a from PBS-injected (1 μL) left eye (−) or IVS20×-21, Ex21×+1, IVS21×+18, IVS21×+33 or Cep290 (negative control ASO; Gerard et al, Mol. Ther. Nuc. Ac., 2015) 2′-MOE ASO-injected (1 μL) right eye (+) at 10 mM concentration are shown. Ex21×+1, IVS21×+18, and IVS21×+33 (mouse nomenclature) and Ex20×+1, IVS20×+18, and IVS20×+33 (human nomenclature) are identical. Two products corresponding to exon 21× inclusion (top band) and full-length (exon 21× exclusion, bottom band) were quantified.

FIG. 9B depicts a graph plotting the percent exon 21× inclusion from the data in FIG. 9A. White bars correspond to ASO-injected eyes and grey bars correspond to PBS-injected eyes, n=5 in each group.

FIG. 9C depicts a graph of the full-length products were normalized to Gapdh internal control and fold-change of ASO-injected eye relative to PBS-injected eye is plotted. The black line indicates a ratio of 1 and no change with respect to PBS, n=5 in each group.

FIG. 10A depicts exemplary results from intracerebroventricular (ICV) injection of selected ASOs in C57BL6J mice (male, 3 months old). PAGE gels of SYBR-safe-stained RT-PCR products of mouse Scn1a from uninjected (−, no ASO control), or 300 μg of Cep290 (negative control ASO; Gerard et al, Mol. Ther. Nuc. Ac., 2015), Ex21×+1, IVS21×+18, IVS21×+33 2′-MOE ASO-injected brains are shown. Ex21×+1, IVS21×+18, and IVS21×+33 (mouse nomenclature) and Ex20×+1, IVS20×+18, and IVS20×+33 (human nomenclature) are identical. Two products corresponding to exon 21× inclusion (top band) and full-length (exon 21× exclusion, bottom band) were quantified.

FIG. 10B depicts a graph plotting the percent exon 21× inclusion from the data in FIG. 10A, n=6 (each targeting ASO), n=5 (Cep290 ASO), n=1 (uninjected, no ASO control).

FIG. 10C depicts a graph from results of a Tagman qPCR assay performed using two different probes spanning exons 21 and 22 junction. The products were normalized to Gapdh internal control and fold-change of ASO-injected relative to Cep290-injected brains is plotted. The black line indicates a ratio of 1 and no change with respect to Cep290, n=6 (each targeting ASO), n=5 (Cep290 ASO), n=1 (uninjected, no ASO control).

FIG. 11A depicts exemplary results from intracerebroventricular (ICV) injection of selected ASOs in C57BL6J mice (male, 3 months old). PAGE gels of SYBR-safe-stained RT-PCR products of mouse Scn1a from 300 ug of Cep290 (negative control ASO; Gerard et al, Mol. Ther. Nuc. Ac., 2015), or 33 ug, 100 ug, and 300 ug of Ex21×+1 2′-MOE ASO-injected brains. Ex21×+1 (mouse nomenclature) and Ex20×+1, (human nomenclature) are identical. Two products corresponding to exon 21× inclusion (top band) and full-length (exon 21× exclusion, bottom band) were quantified.

FIG. 11B depicts a graph plotting the percent exon 21× inclusion from the data in FIG. 11A, n=5 (each group).

FIG. 11C depicts a graph from results of a Tagman qPCR assay performed using two different probes spanning exons 21 and 22 junction. The products were normalized to Gapdh internal control and fold-change of ASO-injected relative to Cep290-injected brains is plotted. The black line indicates a ratio of 1 and no change with respect to Cep290, n=5 (each group).

FIG. 12A depicts exemplary results from intracerebroventricular (ICV) injection of a selected ASO in C57BL6J mice (postnatal day 2). PAGE gels of SYBR-safe-stained RT-PCR products of mouse Scn1a from uninjected (−, no ASO control), or 20 μg Ex21×+1 2′-MOE ASO-injected brains are shown. Two products corresponding to exon 21× inclusion (top band) and full-length (exon 21× exclusion, bottom band) were quantified. Ex21×+1 (mouse nomenclature) and Ex20×+1 (human nomenclature) are identical.

FIG. 12B depicts a graph plotting the percent exon 21× inclusion from the data in FIG. 12A, n=4 (each group).

FIG. 12C depicts a graph from results of a Tagman qPCR assay performed using two different probes spanning exons 21 and 22 junction. The products were normalized to Gapdh internal control and fold-change of ASO-injected relative to no-ASO-control brains is plotted.

The black line indicates a ratio of 1 and no change with respect to no-ASO control, n=4 (each group).

FIG. 13A depicts a graph plotting the percent exon 21× inclusion in the indicated mouse CNS samples.

FIG. 13B depicts a graph plotting the percent exon 20× inclusion in the indicated human CNS samples.

FIG. 14A depicts a graph plotting the percent decrease in exon 21× inclusion at the indicated doses.

FIG. 14B depicts a graph plotting the percent increase in Scn1a mRNA at the indicated doses.

FIG. 14C depicts a graph plotting the percent increase in Nav 1.1 protein levels at the indicated doses.

FIG. 15A depicts a graph plotting the percent decrease in exon 21× inclusion at the indicated doses.

FIG. 15B depicts a graph plotting the percent increase in Scn1a mRNA at the indicated doses.

FIG. 16 depicts a selected Scn1a targeting ASO administered at a 10 ug dose via ICV injection in postnatal day 2 mice evaluated at day 5 post-injection by Tagman qPCR of SCN1A, SCN2A, SCN3A, SCN4A, SCN5A, SCN7A, SCN8A, SCN9A, SCN10A, and SCN11A to assess target selectivity. Tagman-qPCR amplification results normalized to Gapdh, obtained using Ex20×+1 ASO, are plotted as fold change relative to PBS injected mice.

FIGS. 17A and 17B depict exemplary results from intracerebroventricular (ICV) injection at postnatal day 2 of a selected ASO at the indicated dose in wild type (WT) or heterozygous Dravet mice (HET) F1 mice from 129S-Scn1a^{tm1Kea}×C57BL/6J crosses at 3 days post-injection. FIG. 17A depicts a graph from results of a Tagman qPCR assay performed using a probe spanning exons 21 and 22. The products were normalized to Gapdh internal control and fold-change of ASO-injected relative to PBS-injected brains is plotted. FIG. 17B depicts a graph from results of a western blot performed using an anti-Nav1.1 antibody. The products were normalized to Ponceau-stained bands and fold-change of ASO-injected relative to PBS-injected brains is plotted.

FIG. 18 depicts exemplary results of a SCN1A exon 20× region ASO microwalk in RenCells via free uptake. ASOs were designed to cover regions around three previously identified targeting ASOs in FIG. 6 (marked by stars) by shifting 1 nucleotides at a time (6-41) or by decreasing the length of ASO 17 (1-5). The graph depicts the percent exon 20× inclusion as measured by SYBR-green qPCR. The black line indicates no change with respect to no ASO (−).

FIG. 19 is a graph plotting increase in Scn1a mRNA level in coronal brain slices of mice over the time post injection of a SCN1A targeting ASO. As depicted, increase in Scn1a mRNA level was maintained for at least 80 days post-injection.

FIG. 20 is an exemplary survival curve demonstrating 100% survival benefit provided by a SCN1A targeting ASO in Dravet mouse model. +/+ stands for WT genotype, and +/− stands for 129S-scn1a^{tm1Kea} heterozygous genotype (Dravet mouse model); A stands for PBS treatment, and B stands for ASO treatment. As depicted, mice in A +/− group (Dravet mice receiving PBS treatment) started to die from about postnatal day 16, whereas all mice of other three groups, including B +/−(Drave mice receiving ASO treatment) group, survived through at least postnatal day 35.

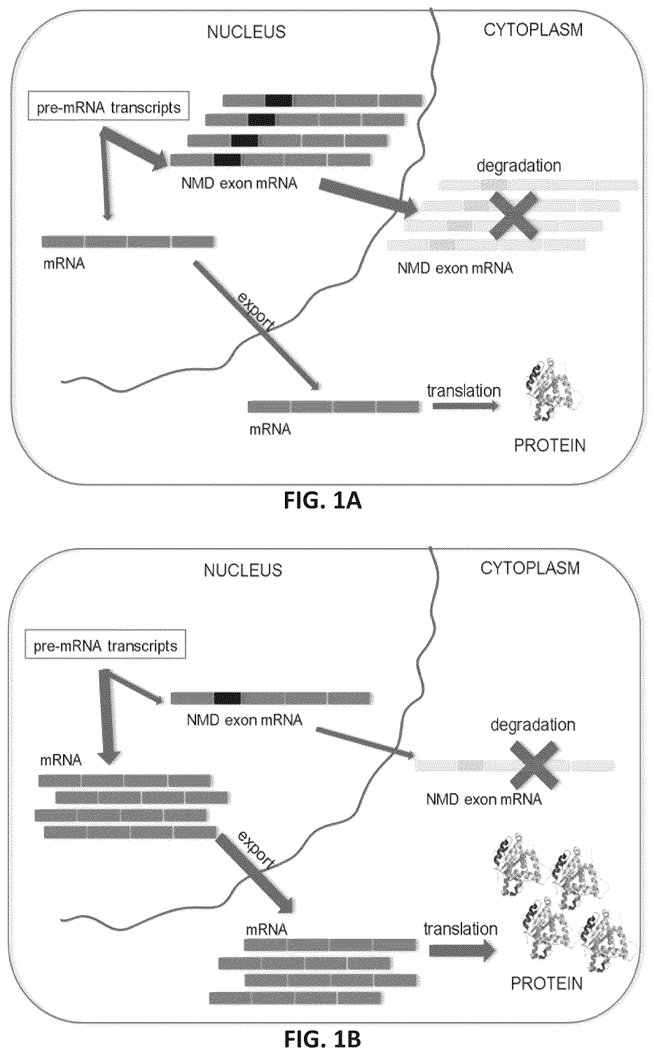

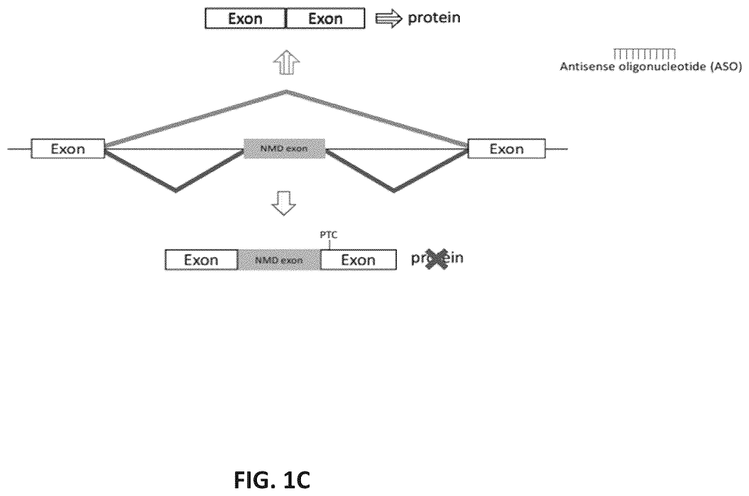

### DETAILED DESCRIPTION OF THE INVENTION

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins.

Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD.

NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

A NMD-inducing exon (NIE) is an exon or a pseudo-exon that is a region within an intron and can activate the NMD pathway if included in a mature RNA transcript. In the constitutive splicing events, the intron containing an NIE is usually spliced out, but the intron or a portion thereof (e.g. NIE) can be retained during alternative or aberrant splicing events. Mature mRNA transcripts containing such an NIE can be non-productive due to frame shift which induce NMD pathway. Inclusion of a NIE in mature RNA transcripts can downregulate gene expression. mRNA transcripts containing an NIE can be referred as “NIE containing mRNA” or “NMD exon mRNA” in the current disclosure.

Cryptic (or pseudo-splice sites) have the same splicing recognition sequences as genuine splice sites but are not used in the splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (http://www.ncbi.nlm.nih.gov/pmc/articles/PMC2095810/pdf/gkm680.pdf)

The cryptic splice sites or splicing regulatory sequences may compete for RNA-binding proteins such as U2AF with a splice site of the NIE. In one embodiment, an agent may bind to the cryptic splice site or splicing regulatory sequences to prevent the binding of RNA-binding proteins and thereby favoring utilization of the NIE splice sites.

In one embodiment, the cryptic splice site may not comprise the 5′ or 3′ splice site of the NIE. The cryptic splice site may be at least 10 nucleotides upstream of the NIE 5′ splice site.

The cryptic splice site may be at least 20 nucleotides upstream of the NIE 5′ splice site. The cryptic splice site may be at least 50 nucleotides upstream of the NIE 5′ splice site. The cryptic splice site may be at least 100 nucleotides upstream of the NIE 5′ splice site. The cryptic splice site may be at least 200 nucleotides upstream of the NIE 5′ splice site.

The cryptic splice site may be at least 10 nucleotides downstream of the NIE 3′ splice site. The cryptic splice site may be at least 20 nucleotides downstream of the NIE 3′ splice site.

The cryptic splice site may be at least 50 nucleotides downstream of the NIE 3′ splice site. The cryptic splice site may be at least 100 nucleotides downstream of the NIE 3′ splice site. The cryptic splice site may be at least 200 nucleotides downstream of the NIE 3′ splice site.

### Target Transcripts

In some embodiments, the methods of the present disclosure exploit the presence of NIE in the pre-mRNA transcribed from the SCN1A gene. Splicing of the identified SCN1A NIE pre-mRNA species to produce functional mature SCN1A mRNA can be induced using a therapeutic agent such as an ASO that stimulates exon skipping of an NIE. Induction of exon skipping can result in inhibition of an NMD pathway. The resulting mature SCN1A mRNA can be translated normally without activating NMD pathway, thereby increasing the amount of SCN1A protein in the patient's cells and alleviating symptoms of a condition associated with SCN1A deficiency, such as Dravet Syndrome (DS); Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP.

In various embodiments, the present disclosure provides a therapeutic agent which can target SCN1A mRNA transcripts to modulate, e.g., enhance or inhibit, splicing or protein expression level. The therapeutic agent can be a small molecule, polynucleotide, or polypeptide. In some embodiments, the therapeutic agent is an ASO. Various regions or sequences on the SCN1A pre-mRNA can be targeted by a therapeutic agent, such as an ASO. In some embodiments, the ASO targets a SCN1A pre-mRNA transcript containing an NIE. In some embodiments, the ASO targets a sequence within an NIE of a SCN1A pre-mRNA transcript. In some embodiments, the ASO targets a sequence upstream (or 5′) from the 5′ end of an NIE (3′ss) of a SCN1A pre-mRNA transcript. In some embodiments, the ASO targets a sequence downstream (or 3′) from the 3′ end of an NIE (5′ss) of a SCN1A pre-mRNA transcript. In some embodiments, the ASO targets a sequence that is within an intron flanking on the 5′ end of the NIE of a SCN1A pre-mRNA transcript. In some embodiments, the ASO targets a sequence that is within an intron flanking the 3′ end of the NIE of a SCN1A pre-mRNA transcript. In some embodiments, the ASO targets a sequence comprising an NIE-intron boundary of a SCN1A pre-mRNA transcript. An NIE-intron boundary can refer to the junction of an intron sequence and an NIE region. The intron sequence can flank the 5′ end of the NIE, or the 3′ end of the NIE. In some embodiments, the ASO targets a sequence within an exon of a SCN1A pre-mRNA transcript. In some embodiments, the ASO targets a sequence within an intron of a SCN1A pre-mRNA transcript. In some embodiments, the ASO targets a sequence comprising both a portion of an intron and a portion of an exon.

In some embodiments, a therapeutic agent described herein modulates binding of a factor involved in splicing of the NMD exon mRNA.

In some embodiments, a therapeutic agent described herein interferes with binding of a factor involved in splicing of the NMD exon mRNA.

In some embodiments, a therapeutic agent described herein prevents binding of a factor involved in splicing of the NMD exon mRNA.

In some embodiments, a therapeutic agent targets a targeted portion located in an intronic region between two canonical exonic regions of the NMD exon mRNA encoding SCN1A, and wherein the intronic region contains the NMD exon.

In some embodiments, a therapeutic agent targets a targeted portion at least partially overlaps with the NMD exon.

In some embodiments, a therapeutic agent targets a targeted portion that is at least partially overlaps with an intron upstream of the NMD exon.

In some embodiments, a therapeutic agent targets a targeted portion within the NMD exon.

In some embodiments, a therapeutic agent targets a targeted portion comprising at least about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, a therapeutic agent targets a targeted portion comprising at most about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, a therapeutic agent targets a targeted portion comprising about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, a therapeutic agent targets a targeted portion proximal to the NMD exon.

In some embodiments, the ASO targets a sequence from about 4 to about 300 nucleotides upstream (or 5′) from the 5′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from the 5′ end of the NIE region. In some embodiments, the ASO may target a sequence more than 300 nucleotides upstream from the 5′ end of the NIE. In some embodiments, the ASO targets a sequence from about 4 to about 300 nucleotides downstream (or 3′) from the 3′ end of the NIE.

In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from the 3′ end of the NIE. In some embodiments, the ASO targets a sequence more than 300 nucleotides downstream from the 3′ end of the NIE.

In some embodiments, the ASO targets a sequence from about 4 to about 300 nucleotides upstream (or 5′) from the 5′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from the 5′ end of the NIE region. In some embodiments, the ASO targets a sequence about 4 to about 300 nucleotides downstream (or 3′) from the 3′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from the 3′ end of the NIE. In some embodiments, the ASO targets a sequence more than 300 nucleotides downstream from the 3′ end of the NIE.

In some embodiments, the ASO targets a sequence from about 4 to about 300 nucleotides upstream (or 5′) from the 5′ end of the NIE. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from the 5′ end of the NIE region. In some embodiments, the ASO targets a sequence about 4 to about 300 nucleotides downstream (or 3′) from the 3′ end of the NIE. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from the 3′ end of the NIE. In some embodiments, the ASO targets a sequence more than 300 nucleotides downstream from the 3′ end of the NIE.

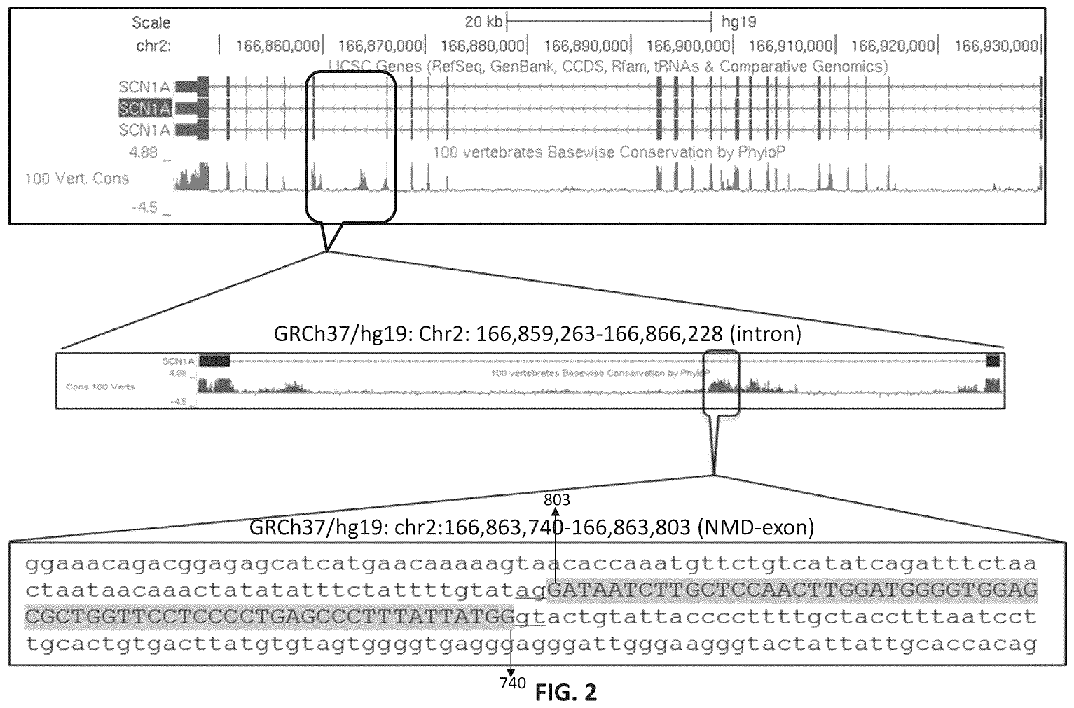

In some embodiments, the NIE as described herein is located between GRCh37/hg19: chr2:166,863,740 and GRCh37/hg19: chr2:166,863,803, as depicted in FIG. 2. In some embodiments, the 5′ end of the NIE is located at GRCh37/hg19: chr2:166,863,803. In some embodiments, the 3′ end of the NIE is located at GRCh37/hg19: chr2:166,863,740.

In some embodiments, In some embodiments, the ASO targets a sequence from about 4 to about 300 nucleotides upstream (or 5′) from genomic site GRCh37/hg19: chr2:166,863,803.

In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from genomic site GRCh37/hg19: chr2:166,863,803. In some embodiments, the ASO may target a sequence more than 300 nucleotides upstream from genomic site GRCh37/hg19: chr2:166,863,803. In some embodiments, the ASO targets a sequence from about 4 to about 300 nucleotides downstream (or 3′) from GRCh37/hg19: chr2:166,863,740. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from GRCh37/hg19: chr2:166,863,740. In some embodiments, the ASO targets a sequence more than 300 nucleotides downstream from GRCh37/hg19: chr2:166,863,740.

In some embodiments, the ASO targets a sequence from about 4 to about 300 nucleotides upstream (or 5′) from genomic site GRCh37/hg19: chr2:166,863,803. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from genomic site GRCh37/hg19: chr2:166,863,803. In some embodiments, the ASO targets a sequence from about 4 to about 300 nucleotides downstream (or 3′) from GRCh37/hg19: chr2:166,863,740. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from GRCh37/hg19: chr2:166,863,740. In some embodiments, the ASO targets a sequence more than 300 nucleotides downstream from GRCh37/hg19: chr2:166,863,740.

In some embodiments, the ASO targets a sequence from about 4 to about 300 nucleotides upstream (or 5′) from genomic site GRCh37/hg19: chr2:166,863,803. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from genomic site GRCh37/hg19: chr2:166,863,803. In some embodiments, the ASO targets a sequence from about 4 to about 300 nucleotides downstream (or 3′) from GRCh37/hg19: chr2:166,863,740. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from GRCh37/hg19: chr2:166,863,740. In some embodiments, the ASO targets a sequence more than 300 nucleotides downstream from GRCh37/hg19: chr2:166,863,740.

As described herein in the Examples, the SCN1A gene (SEQ ID NO. 1) was analyzed for NIE and inclusion of a portion of intron 20 (SEQ ID NO. 4) (this portion is referred as exon 20× throughout the present disclosure) was observed. In some embodiments, the ASOs disclosed herein target a NIE containing pre-mRNA (SEQ ID NO. 2) transcribed from a SCN1A genomic sequence. In some embodiments, the ASO targets a NIE containing pre-mRNA transcript from a SCN1A genomic sequence comprising a portion of intron 20. In some embodiments, the ASO targets a NIE containing pre-mRNA transcript from a SCN1A genomic sequence comprising exon 20× (SEQ ID NO. 6). In some embodiments, the ASO targets a NIE containing pre-mRNA transcript of SEQ ID NO. 2 or 12. In some embodiments, the ASO targets a NIE containing pre-mRNA transcript of SEQ ID NO. 2 or 12 comprising an NIE. In some embodiments, the ASO targets a NIE containing pre-mRNA transcript of SEQ ID NO. 2 comprising exon 20× (SEQ ID NO. 10). In some embodiments, the ASOs disclosed herein target a SCN1A pre-mRNA sequence (SEQ ID NO. 2 or 12). In some embodiments, the ASO targets a SCN1A pre-mRNA sequence comprising an NIE (SEQ ID NO. 10 or 20). In some embodiments, the ASO targets a SCN1A pre-mRNA sequence according to any one of SEQ ID NOs: 7-10 or 17-20. In some embodiments, the ASO has a sequence according to any one of SEQ ID NOs: 21-67. In some embodiments, the ASO has a sequence according to any one of SEQ ID NOs: 68-114. In some embodiments, the ASO has a sequence according to any one of SEQ ID NOs: 115-209. In some embodiments, the ASO has a sequence according to any one of SEQ ID NOs: 210-256. In some embodiments, the ASO has a sequence according to any one of SEQ ID NOs: 257-303. In some embodiments, the ASO has a sequence according to any one of SEQ ID NOs: 304-341. In some embodiments, the ASO has a sequence according to any one of SEQ ID NOs: 342-379.

In some embodiments, the SCN1A NIE containing pre-mRNA transcript is encoded by a genetic sequence with at least about 80%, 85%, 90%, 95%, 96%, 97%, 98%, 99% or 100% sequence identity to SEQ ID NO.: 1 or 11. In some embodiments, the SCN1A NIE pre-mRNA transcript comprises a sequence with at least about 80%, 85%, 90%, 95%, 96%, 97%, 98%, 99% or 100% sequence identity to any one of SEQ ID NOs.: 2-10 and 12-20.

In some embodiments, the SCN1A NIE containing pre-mRNA transcript (or NMD exon mRNA) comprises a sequence with at least about 80%, 85%, 90%, 95%, 97%, or 100% sequence identity to any one of SEQ ID NOs: 2, 7-10, 12, and 17-20. In some embodiments, SCN1A NIE containing pre-mRNA transcript (or NMD exon mRNA) is encoded by a sequence with at least about 80%, 85%, 90%, 95%, 97%, or 100% sequence identity to SEQ ID NOs: 1, 3-6, 11, and 13-16. In some embodiments, the targeted portion of the NMD exon mRNA comprises a sequence with at least 80%, 85%, 90%, 95%, 97%, or 100% sequence identity to a region comprising at least 8 contiguous nucleic acids of SEQ ID NOs: 2, 7-10, 12, and 17-20.

In some embodiments, the ASO targets exon 20 of a SCN1A NIE containing pre-mRNA comprising NIE exon 20×. In some embodiments, the ASO targets an exon 21 sequence downstream (or 3′) of NIE exon 20×. In some embodiments, the ASO targets a sequence about 4 to about 300 nucleotides upstream (or 5′) from the 5′ end of exon 20×. In some embodiments, the ASO targets a sequence about 4 to about 300 nucleotides downstream (or 3′) from the 3′ end of exon 20×. In some embodiments, the ASO has a sequence according to any one of SEQ ID NOs: 21-67. In some embodiments, the ASO has a sequence according to any one of SEQ ID NOs: 210-256.

In some embodiments, the ASO targets a sequence upstream from the 5′ end of an NIE. For example, ASOs targeting a sequence upstream from the 5′ end of an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 21-38. For another example, ASOs targeting a sequence upstream from the 5′ end of an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 68-85. In some embodiments, the ASOs target a sequence containing a exon-intron boundary (or junction). For example, ASOs targeting a sequence containing an exon-intron boundary can comprise a sequence with at least 80%, 85%, 90%, 95%, 97%, or 100% sequence identity to any one of SEQ ID NOs: 39-41, 51, 52, 228-230, 240, or 241. For another example, ASOs targeting a sequence containing an exon-intron boundary can comprise a sequence with at least 80%, 85%, 90%, 95%, 97%, or 100% sequence identity to any one of SEQ ID NOs: 86-88 and 98-99. In some embodiments, the ASOs target a sequence downstream from the 3′ end of an NIE. For example, ASOs targeting a sequence downstream from the 3′ end of an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 53-67. For another example, ASOs targeting a sequence downstream from the 3′ end of an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 100-114. In some embodiments, ASOs target a sequence within an NIE. For example, ASOs targeting a sequence within an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 42-50, or 231-239. For another example, ASOs targeting a sequence within an NIE (e.g. exon 20× in human SCN1A, or exon 21× in mouse SCN1A) can comprise a sequence with at least 80%, 85%, 90%, 95%, 97%, or 100% sequence identity to any one of SEQ ID NOs: 89-97.

In some embodiments, the ASO targets exon 20× in a SCN1A NIE containing pre-mRNA comprising exon 20×. In some embodiments, the ASO targets an exon 20× sequence downstream (or 3′) from the 5′ end of the exon 20× of a SCN1A pre-mRNA. In some embodiments, the ASO targets an exon 20× sequence upstream (or 5′) from the 3′ end of the exon 20× of a SCN1A pre-mRNA.

In some embodiments, the targeted portion of the SCN1A NIE containing pre-mRNA is in intron 1, 2, 3, 4, 5, 6, 7, 8, 9, 10, 11, 12, 13, 14, 15, 16, 17, 18, 19, 20, 21, 22, 23, 24, or 25 (intron numbering corresponding to the mRNA sequence at NM_006920). In some embodiments, hybridization of an ASO to the targeted portion of the NIE pre-mRNA results in exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently increases SCN1A protein production.

In some embodiments, hybridization of an ASO to the targeted portion of the NIE pre-mRNA inhibits or blocks exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently decreases SCN1A protein production. In some embodiments, the targeted portion of the SCN1A NIE containing pre-mRNA is in intron 20. One of skill in the art can determine the corresponding intron number in any isoform based on an intron sequence provided herein or using the number provided in reference to the mRNA sequence at NM_006920, NM_001202435, NM_001165964, or NM_001165963. One of skill in the art also can determine the sequences of flanking exons in any SCN1A isoform for targeting using the methods of the invention, based on an intron sequence provided herein or using the intron number provided in reference to the mRNA sequence at NM_006920, NM_001202435, NM_001165964, or NM_001165963.

In some embodiments, the methods and compositions of the present disclosure are used to modulate, e.g., increase or decrease, the expression of SCN1A by inducing or inhibiting exon skipping of a pseudo-exon of an SCN1A NIE containing pre-mRNA. In some embodiments, the pseudo-exon is a sequence within any of introns 1-25. In some embodiments, the pseudo-exon is a sequence within any of introns 2, 4, 6, 13, 14, 15, 16, 17, 18, 20, 21, 22, 23, 24, and 25. In some embodiments, the pseudo-exon is a sequence within any of introns 15, 18, and 19. In some embodiments, the pseudo-exon can be any SCN1A intron or a portion thereof. In some embodiments, the pseudo-exon is within intron 20. The SCN1A intron numbering used herein corresponds to the mRNA sequence at NM_006920. It is understood that the intron numbering may change in reference to a different SCN1A isoform sequence.

### SCN1A Protein

The SCN1A gene can encode SCN1A (sodium channel, voltage-gated, type I, alpha subunit) protein, which can also be referred to as alpha-subunit of voltage-gated sodium channel Nay1.1. Also described above, SCN1A mutations in DS are spread across the entire protein. More than 100 novel mutations have been identified throughout the gene with the more debilitating arising de novo. These comprise of truncations (47%), missense (43%), deletions (3%), and splice site mutations (7%). The percentage of subjects carrying SCN1A mutations varies between 33 and 100%. The majority of mutations are novel changes (88%).

In some embodiments, the methods described herein are used to modulate, e.g., increase or decrease, the production of a functional SCN1A protein. As used herein, the term “functional” refers to the amount of activity or function of a SCN1A protein that is necessary to eliminate any one or more symptoms of a treated condition, e.g., Dravet syndrome; Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP. In some embodiments, the methods are used to increase the production of a partially functional SCN1A protein. As used herein, the term “partially functional” refers to any amount of activity or function of the SCN1A protein that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SCN1A protein by cells of a subject having a NIE containing pre-mRNA encoding the SCN1A protein, wherein the subject has Dravet syndrome caused by a deficient amount of activity of SCN1A protein, and wherein the deficient amount of the SCN1A protein is caused by haploinsufficiency of the SCN1A protein. In such an embodiment, the subject has a first allele encoding a functional SCN1A protein, and a second allele from which the SCN1A protein is not produced. In another such embodiment, the subject has a first allele encoding a functional SCN1A protein, and a second allele encoding a nonfunctional SCN1A protein. In another such embodiment, the subject has a first allele encoding a functional SCN1A protein, and a second allele encoding a partially functional SCN1A protein. In any of these embodiments, the antisense oligomer binds to a targeted portion of the NIE containing pre-mRNA transcribed from the second allele, thereby inducing exon skipping of the pseudo-exon from the pre-mRNA, and causing an increase in the level of mature mRNA encoding functional SCN1A protein, and an increase in the expression of the SCN1A protein in the cells of the subject.

In related embodiments, the method is a method of using an ASO to increase the expression of a protein or functional RNA. In some embodiments, an ASO is used to increase the expression of SCN1A protein in cells of a subject having a NIE containing pre-mRNA encoding SCN1A protein, wherein the subject has a deficiency, e.g., Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or autism, in the amount or function of a SCN1A protein. In some embodiments, an ASO is used to increase the expression of SCN1A protein in cells of a subject, wherein the subject has a deficiency, e.g., Epileptic encephalopathy, early infantile, 13; in the amount or function of a SCN8A protein. In some embodiments, an ASO is used to increase the expression of SCN1A protein in cells of a subject, wherein the subject has a deficiency, e.g., Sick sinus syndrome 1; in the amount or function of a SCN5A protein.

In some embodiments, the NIE containing pre-mRNA transcript that encodes the protein that is causative of the disease or condition is targeted by the ASOs described herein. In some embodiments, a NIE containing pre-mRNA transcript that encodes a protein that is not causative of the disease is targeted by the ASOs. For example, a disease that is the result of a mutation or deficiency of a first protein in a particular pathway may be ameliorated by targeting a NIE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele from which
   (i) the SCN1A protein is produced at a reduced level compared to production from a wild-type allele,
   (ii) the SCN1A protein is produced in a form having reduced function compared to an equivalent wild-type protein, or
   (iii) the SCN1A protein or functional RNA is not produced; and
(b) a second mutant allele from which
   (i) the SCN1A protein is produced at a reduced level compared to production from a wild-type allele,
   (ii) the SCN1A protein is produced in a form having reduced function compared to an equivalent wild-type protein, or
   (iii) the SCN1A protein is not produced, and
      wherein the NIE containing pre-mRNA is transcribed from the first allele and/or the second allele. In these embodiments, the ASO binds to a targeted portion of the NIE containing pre-mRNA transcribed from the first allele or the second allele, thereby inducing exon skipping of the pseudo-exon from the NIE containing pre-mRNA, and causing an increase in the level of mRNA encoding SCN1A protein and an increase in the expression of the target protein or functional RNA in the cells of the subject. In these embodiments, the target protein or functional RNA having an increase in expression level resulting from the exon skipping of the pseudo-exon from the NIE containing pre-mRNA is either in a form having reduced function compared to the equivalent wild-type protein (partially-functional), or having full function compared to the equivalent wild-type protein (fully-functional).

In some embodiments, the level of mRNA encoding SCN1A protein is increased 1.1 to 10-fold, when compared to the amount of mRNA encoding SCN1A protein that is produced in a control cell, e.g., one that is not treated with the antisense oligomer or one that is treated with an antisense oligomer that does not bind to the targeted portion of the SCN1A NIE containing pre-mRNA.

In some embodiments, a subject treated using the methods of the present disclosure expresses a partially functional SCN1A protein from one allele, wherein the partially functional SCN1A protein is caused by a frameshift mutation, a nonsense mutation, a missense mutation, or a partial gene deletion. In some embodiments, a subject treated using the methods of the invention expresses a nonfunctional SCN1A protein from one allele, wherein the nonfunctional SCN1A protein is caused by a frameshift mutation, a nonsense mutation, a missense mutation, a partial gene deletion, in one allele. In some embodiments, a subject treated using the methods of the invention has a SCN1A whole gene deletion, in one allele.

In some embodiments, the method is a method of decreasing the expression of the SCN1A protein by cells of a subject having a NIE containing pre-mRNA encoding the SCN1A protein, and wherein the subject has a gain-of-function mutation in Nay1.1. In such an embodiment, the subject has an allele from which the SCN1A protein is produced in an elevated amount or an allele encoding a mutant SCN1A that induces increased activity of Nay1.1 in the cell. In some embodiments, the increased activity of Nay1.1 is characterized by a prolonged or near persistent sodium current mediated by the mutant Nay1.1 channel, a slowing of fast inactivation, a positive shift in steady-state inactivation, higher channel availability during repetitive stimulation, increased non-inactivated depolarization-induced persistent sodium currents, delayed entry into inactivation, accelerated recovery from fast inactivation, and/or rescue of folding defects by incubation at lower temperature or co-expression of interacting proteins. In any of these embodiments, the antisense oligomer binds to a targeted portion of the NIE containing pre-mRNA transcribed from the second allele, thereby inhibiting or blocking exon skipping of the pseudo-exon from the pre-mRNA, and causing a decrease in the level of mature mRNA encoding functional SCN1A protein, and a decrease in the expression of the SCN1A protein in the cells of the subject.

In related embodiments, the method is a method of using an ASO to decrease the expression of a protein or functional RNA. In some embodiments, an ASO is used to decrease the expression of SCN1A protein in cells of a subject having a NIE containing pre-mRNA encoding SCN1A protein. In some embodiments, the subject has a gain-of-function mutation in Nay1.1, e.g., migraine. In some embodiments, an ASO is used to decrease the expression of SCN1A protein in cells of a subject, the subject has a gain-of-function mutation in Nay1.1, e.g., migraine, familial hemiplegic, 3.

In some embodiments, the level of mRNA encoding SCN1A protein is decreased 1.1 to 10-fold, when compared to the amount of mRNA encoding SCN1A protein that is produced in a control cell, e.g., one that is not treated with the antisense oligomer or one that is treated with an antisense oligomer that does not bind to the targeted portion of the SCN1A NIE containing pre-mRNA.

In some embodiments, a subject treated using the methods of the present disclosure expresses a mutant SCN1A protein from one allele, wherein the mutant SCN1A protein is caused by a frameshift mutation, a nonsense mutation, a missense mutation, or a partial gene deletion, and wherein the mutant SCN1A protein causes an elevated activity level of Nay1.1. In some embodiments, a subject treated using the methods of the present disclosure expresses an elevated amount of SCN1A protein from one allele due to a frameshift mutation, a nonsense mutation, a missense mutation, or a partial gene deletion.

In embodiments of the present invention, a subject can have a mutation in SCN1A. Mutations in SCN1A can be spread throughout said gene. SCN1A protein can consist of four domains. Said SCN1A domains can have transmembrane segments. Mutations in said SCN1A protein may arise throughout said protein. Said SCN1A protein may consist of at least two isoforms. Mutations in SCN1A may comprise of R931C, R946C, M934I, R1648C, or R1648H.

In some cases, mutations may be observed in a C-terminus of a SCN1A protein. Mutations in a SCN1A protein may also be found in loops between segments 5 and 6 of the first three domains of said SCN1A protein. In some cases, mutations may be observed in an N-terminus of a SCN1A protein. Exemplary mutations within SCN1A include, but are not limited to, R222X, R712X, I227S, R1892X, W952X, R1245X, R1407X, W1434R, c.4338+1G>A, S1516X, L1670fsX1678, or K1846fsX1856. Mutations that can be targeted with the present invention may also encode a pore of an ion channel.

In some embodiments, the methods and compositions described herein can be used to treat DS. In other embodiments, the methods and compositions described herein can be used to treat severe myclonic epilepsy of infancy (SMEI). In other embodiments, the methods and compositions described herein can be used to treat borderline Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP. The methods and compositions described herein can also be used to treat borderline SMEI. Additionally, the methods and compositions described herein can be used to treat generalized epilepsy with febrile seizures plus (GEFS+). GEFS+ may be associated with mutations in epilepsy-associated ion channel subunits such as SCN1B or GABRG2. The methods and compositions described herein can also be used to treat sodium channelopathies. Sodium channelopathies may be associated with mutations in SCN1A. Sodium channelopathies may also be associated with subunits of SCN1A, such as the beta subunit, SCN1B. In some cases, additional diseases associated with SCN1A mutations may also be treated with the present disclosure. Related SCN1A diseases associated with SCN1A mutations include, but are not limited to, atypical myotonia congenita, hyperkalemic periodic paralysis, and paramyotonia congenita.

In some embodiments, a subject having any SCN1A mutation known in the art and described in the literature referenced above (e.g., by Hamdan, et al., 2009, Mulley, et al., 2005) can be treated using the methods and compositions described herein. In some embodiments, the mutation is within any SCN1A intron or exon.

### Exon Inclusion

As used herein, a “NIE containing pre-mRNA” is a pre-mRNA transcript that contains at least one pseudo-exon. Alternative or aberrant splicing can result in inclusion of the at least one pseudo-exon in the mature mRNA transcripts. The terms “mature mRNA,” and “fully-spliced mRNA,” are used interchangeably herein to describe a fully processed mRNA. Inclusion of the at least one pseudo-exon can be non-productive mRNA and lead to NMD of the mature mRNA. NIE containing mature mRNA may sometimes lead to aberrant protein expression.

In some embodiments, the included pseudo-exon is the most abundant pseudo-exon in a population of NIE containing pre-mRNAs transcribed from the gene encoding the target protein in a cell. In some embodiments, the included pseudo-exon is the most abundant pseudo-exon in a population of NIE containing pre-mRNAs transcribed from the gene encoding the target protein in a cell, wherein the population of NIE containing pre-mRNAs comprises two or more included pseudo-exons. In some embodiments, an antisense oligomer targeted to the most abundant pseudo-exon in the population of NIE containing pre-mRNAs encoding the target protein induces exon skipping of one or two or more pseudo-exons in the population, including the pseudo-exon to which the antisense oligomer is targeted or binds. In embodiments, the targeted region is in a pseudo-exon that is the most abundant pseudo-exon in a NIE containing pre-mRNA encoding the SCN1A protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included pseudo-exon is an exon that is identified as an included pseudo-exon based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included pseudo-exon is an exon that is identified as a included pseudo-exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying exon inclusion.

In some embodiments, contacting cells with an ASO that is complementary to a targeted portion of a SCN1A pre-mRNA transcript results in an increase in the amount of SCN1A protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SCN1A protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in a decrease in the amount of SCN1A protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SCN1A protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in an increase in the amount of mRNA encoding SCN1A, including the mature mRNA encoding the target protein. In some embodiments, the amount of mRNA encoding SCN1A protein, or the mature mRNA encoding the SCN1A protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SCN1A protein, or the mature mRNA encoding SCN1A protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

In some embodiments, contacting cells with an ASO that is complementary to a targeted portion of a SCN1A pre-mRNA transcript results in a decrease in the amount of mRNA encoding SCN1A, including the mature mRNA encoding the target protein. In some embodiments, the amount of mRNA encoding SCN1A protein, or the mature mRNA encoding the SCN1A protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SCN1A protein, or the mature mRNA encoding SCN1A protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

The NIE can be in any length. In some embodiments, the NIE comprises a full sequence of an intron, in which case, it can be referred to as intron retention. In some embodiments, the NIE can be a portion of the intron. In some embodiments, the NIE can be a 5′ end portion of an intron including a 5′ss sequence. In some embodiments, the NIE can be a 3′ end portion of an intron including a 3′ss sequence. In some embodiments, the NIE can be a portion within an intron without inclusion of a 5′ss sequence. In some embodiments, the NIE can be a portion within an intron without inclusion of a 3′ss sequence. In some embodiments, the NIE can be a portion within an intron without inclusion of either a 5′ss or a 3′ss sequence. In some embodiments, the NIE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NIE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NIE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NIE may be longer than 1,000 nucleotides in length.

Inclusion of a pseudo-exon can lead to a frameshift and the introduction of a premature termination codon (PIC) in the mature mRNA transcript rendering the transcript a target of NMD. Mature mRNA transcript containing NIE can be non-productive mRNA transcript which does not lead to protein expression. The PIC can be present in any position downstream of an NIE. In some embodiments, the PIC can be present in any exon downstream of an NIE. In some embodiments, the PIC can be present within the NIE. For example, inclusion of exon 20× in an mRNA transcript encoded by the SCN1A gene can induce a PIC in the mRNA transcript, e.g., a PIC in exon 21 of the mRNA transcript.

### Therapeutic Agents

In various embodiments of the present disclosure, compositions and methods comprising a therapeutic agent are provided to modulate protein expression level of SCN1A. In some embodiments, provided herein are compositions and methods to modulate alternative splicing of SCNA1 pre-mRNA. In some embodiments, provided herein are compositions and methods to induce exon skipping in the splicing of SCN1A pre-mRNA, e.g., to induce skipping of a pseudo-exon during splicing of SCN1A pre-mRNA. In other embodiments, therapeutic agents may be used to induce the inclusion of an exon in order to decrease the protein expression level.

In some embodiment, a therapeutic agent disclosed herein is a small molecule, a polypeptide, or a polynucleic acid polymer. In some instances, the therapeutic agent is a small molecule. In some instances, the therapeutic agent is a polypeptide. In some instances, the therapeutic agent is a polynucleic acid polymer. In some cases, the therapeutic agent is a repressor agent. In additional cases, the therapeutic agent is an enhancer agent.

A therapeutic agent disclosed herein can be a NIE repressor agent. A therapeutic agent may comprise a polynucleic acid polymer.

According to one aspect of the present disclosure, provided herein is a method of treatment or prevention of a condition associated with a functional-SCN1A protein deficiency, comprising administering a NIE repressor agent to a subject to increase levels of functional SCN1A protein, wherein the agent binds to a region of the pre-mRNA transcript to decrease inclusion of the NIE in the mature transcript. For example, provided herein is a method of treatment or prevention of a condition associated with a functional-SCN1A protein deficiency, comprising administering a NIE repressor agent to a subject to increase levels of functional SCN1A protein, wherein the agent binds to a region of an intron containing an NIE (e.g., intron 20 in human SCN1A gene) of the pre-mRNA transcript or to a NIE-activating regulatory sequence in the same intron.

Where reference is made to reducing NIE inclusion in the mature mRNA, the reduction may be complete, e.g., 100%, or may be partial. The reduction may be clinically significant. The reduction/correction may be relative to the level of NIE inclusion in the subject without treatment, or relative to the amount of NIE inclusion in a population of similar subjects. The reduction/correction may be at least 10% less NIE inclusion relative to the average subject, or the subject prior to treatment. The reduction may be at least 20% less NIE inclusion relative to an average subject, or the subject prior to treatment. The reduction may be at least 40% less NIE inclusion relative to an average subject, or the subject prior to treatment. The reduction may be at least 50% less NIE inclusion relative to an average subject, or the subject prior to treatment. The reduction may be at least 60% less NIE inclusion relative to an average subject, or the subject prior to treatment. The reduction may be at least 80% less NIE inclusion relative to an average subject, or the subject prior to treatment. The reduction may be at least 90% less NIE inclusion relative to an average subject, or the subject prior to treatment.

Where reference is made to increasing active-SCN1A protein levels, the increase may be clinically significant. The increase may be relative to the level of active-SCN1A protein in the subject without treatment, or relative to the amount of active-SCN1A protein in a population of similar subjects. The increase may be at least 10% more active-SCN1A protein relative to the average subject, or the subject prior to treatment. The increase may be at least 20% more active-SCN1A protein relative to the average subject, or the subject prior to treatment. The increase may be at least 40% more active-SCN1A protein relative to the average subject, or the subject prior to treatment. The increase may be at least 50% more active-SCN1A protein relative to the average subject, or the subject prior to treatment. The increase may be at least 80% more active-SCN1A protein relative to the average subject, or the subject prior to treatment. The increase may be at least 100% more active-SCN1A protein relative to the average subject, or the subject prior to treatment. The increase may be at least 200% more active-SCN1A protein relative to the average subject, or the subject prior to treatment. The increase may be at least 500% more active-SCN1A protein relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 21-67. The polynucleic acid polymer may have a sequence with 100% sequence identity to a sequence selected from the group consisting of SEQ ID NOs: 21-67. In some instances, the polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 68-114. In some cases, the polynucleic acid polymer may have a sequence with 100% sequence identity to a sequence selected from the group consisting of SEQ ID NOs: 68-114.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence.

References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a SCN1A NIE containing pre-mRNA. As used herein, the terms “ASO” and “antisense oligomer” are used interchangeably and refer to an oligomer such as a polynucleotide, comprising nucleobases that hybridizes to a target nucleic acid (e.g., a SCN1A NIE containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a NIE containing pre-mRNA. Typically such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity.

In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a targeted portion of a NIE containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In embodiments, the stereochemistry at each of the phosphorus internucleotide linkages of the ASO backbone is random. In embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In embodiments, an ASO used in the methods of the invention, including, but not limited to, any of the ASOs set forth herein in Tables 5 and 6, comprises an ASO having phosphorus internucleotide linkages that are not random. In embodiments, a composition used in the methods of the invention comprises a pure diastereomeric ASO. In embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In embodiments, an ASO used in the methods of the invention, including, but not limited to, any of the ASOs set forth herein in SEQ ID NOs: 21-114, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In embodiments, an ASO used in the methods of the invention, including, but not limited to, any of the ASOs set forth herein in SEQ ID NOs: 21-114, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In embodiments, an ASO used in the methods of the invention, including, but not limited to, any of the ASOs set forth herein in SEQ ID NOs: 21-114, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the invention, including, but not limited to, any of the ASOs set forth herein in SEQ ID NOs: 21-114, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA).

Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SCN1A NIE containing pre-mRNA that is downstream (in the 3′ direction) of the 5′ splice site (or 3′ end of the NIE) of the included exon in a SCN1A NIE containing pre-mRNA (e.g., the direction designated by positive numbers relative to the 5′ splice site). In some embodiments, the ASOs are complementary to a targeted portion of the SCN1A NIE containing pre-mRNA that is within the region about +1 to about +500 relative to the 5′ splice site (or 3′ end) of the included exon. In some embodiments, the ASOs may be complementary to a targeted portion of a SCN1A NIE containing pre-mRNA that is within the region between nucleotides+6 and +496 relative to the 5′ splice site (or 3′ end) of the included exon. In some aspects, the ASOs are complementary to a targeted portion that is within the region about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of a SCN1A NIE containing pre-mRNA that is upstream (in the 5′ direction) of the 5′ splice site (or 3′ end) of the included exon in a SCN1A NIE containing pre-mRNA (e.g., the direction designated by negative numbers relative to the 5′ splice site). In some embodiments, the ASOs are complementary to a targeted portion of the SCN1A NIE containing pre-mRNA that is within the region about −4 to about −270 relative to the 5′ splice site (or 3′end) of the included exon. In some embodiments, the ASOs may be complementary to a targeted portion of a SCN1A NIE containing pre-mRNA that is within the region between nucleotides −1 and −264 relative to the 5′ splice site (or 3′ end) of the included exon. In some aspects, the ASOs are complementary to a targeted portion that is within the region about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon. In some aspects, the ASOs are complementary to a targeted portion that is within the region from about −1 to about −50, from about −50 to about −100, from about −100 to about −150, from about −150 to about −200, or from about −200 to about −250 relative to 5′ splice site (or 3′ end) of the included exon.

In some embodiments, the ASOs are complementary to a targeted region of a SCN1A NIE containing pre-mRNA that is upstream (in the 5′ direction) of the 3′ splice site (or 5′ end) of the included exon in a SCN1A NIE containing pre-mRNA (e.g., in the direction designated by negative numbers). In some embodiments, the ASOs are complementary to a targeted portion of the SCN1A NIE containing pre-mRNA that is within the region about −1 to about −500 relative to the 3′ splice site (or 5′ end) of the included exon. In some embodiments, the ASOs are complementary to a targeted portion of the SCN1A NIE containing pre-mRNA that is within the region −1 to −496 relative to the 3′ splice site of the included exon. In some aspects, the ASOs are complementary to a targeted portion that is within the region about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, or about −1 to about −30 relative to 3′ splice site of the included exon.

In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of a SCN1A NIE containing pre-mRNA that is downstream (in the 3′ direction) of the 3′ splice site (5′ end) of the included exon in a SCN1A NIE containing pre-mRNA (e.g., in the direction designated by positive numbers). In some embodiments, the ASOs are complementary to a targeted portion of the SCN1A NIE containing pre-mRNA that is within the region of about +1 to about +100 relative to the 3′ splice site of the included exon. In some aspects, the ASOs are complementary to a targeted portion that is within the region about +1 to about +90, about +1 to about +80, about +1 to about +70, about +1 to about +60, about +1 to about +50, about +1 to about +40, about +1 to about +30, about +1 to about +20, or about +1 to about +10 relative to 3′ splice site of the included exon.

In some embodiments, the targeted portion of the SCN1A NIE containing pre-mRNA is within the region+100 relative to the 5′ splice site (3′ end) of the included exon to −100 relative to the 3′ splice site (5′ end) of the included exon. In some embodiments, the targeted portion of the SCN1A NIE containing pre-mRNA is within the NIE. In some embodiments, the targeted portion of the SCN1A NIE containing pre-mRNA comprises a pseudo-exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the NIE containing pre-mRNA are used. In some embodiments, two or more ASOs that are complementary to different targeted portions of the NIE containing pre-mRNA are used.

In embodiments, the antisense oligonucleotides of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a SCN1A NIE containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present invention includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In embodiments, a surfactant is included in the pharmaceutical formulation or compositions. The use of surfactants in drug products, formulations and emulsions is well known in the art. In embodiments, the present invention employs a penetration enhancer to effect the efficient delivery of the antisense oligonucleotide, e.g., to aid diffusion across cell membranes and/or enhance the permeability of a lipophilic drug. In embodiments, the penetration enhancers are a surfactant, fatty acid, bile salt, chelating agent, or non-chelating nonsurfactant.

In embodiments, the pharmaceutical formulation comprises multiple antisense oligonucleotides. In embodiments, the antisense oligonucleotide is administered in combination with another drug or therapeutic agent.

### Combination Therapies

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention. In some embodiments, the one or more other agents are selected from the ASOs listed in Table 1a or Table 1b.

TABLE 1a
Exemplary ASOs to correct intron retention

| SEQ ID | | | Retained |
|---|---|---|---|
| NO: | Name | Sequence (5′-3′) | Intron |
| 115 | SCN1A-IVS21+6 | CAGAGAAAAUAGUGUUCA | 21 |
| 116 | SCN1A-IVS21+11 | AUAUUCAGAGAAAAUAGU | 21 |
| 117 | SCN1A-IVS21+16 | UAAAAAUAUUCAGAGAAA | 21 |
| 118 | SCN1A-IVS21+21 | AACAAUAAAAAUAUUCAG | 21 |
| 119 | SCN1A-IVS21+26 | UUCCAAACAAUAAAAAUA | 21 |
| 120 | SCN1A-IVS21+31 | UAUUAUUCCAAACAAUAA | 21 |
| 121 | SCN1A-IVS21+36 | UUUGUUAUUAUUCCAAAC | 21 |
| 122 | SCN1A-IVS21+41 | AUUAUUUUGUUAUUAUUC | 21 |
| 123 | SCN1A-IVS21+46 | AUGUCAUUAUUUUGUUAU | 21 |
| 124 | SCN1A-IVS21+51 | GAUGUAUGUCAUUAUUUU | 21 |
| 125 | SCN1A-IVS21+56 | UAAUAGAUGUAUGUCAUU | 21 |
| 126 | SCN1A-IVS21+61 | CUAAAUAAUAGAUGUAUG | 21 |
| 127 | SCN1A-IVS21+66 | AGGAACUAAAUAAUAGAU | 21 |
| 128 | SCN1A-IVS21+71 | UUCUUAGGAACUAAAUAA | 21 |
| 129 | SCN1A-IVS21+76 | ACUUUUUCUUAGGAACUA | 21 |
| 130 | SCN1A-IVS21+81 | UAUAUACUUUUUCUUAGG | 21 |
| 131 | SCN1A-IVS21−16 | UGCAUGUUUUACUUUGGA | 21 |
| 132 | SCN1A-IVS21−21 | GUUUUACUUUGGAGUAAA | 21 |
| 133 | SCN1A-IVS21−26 | ACUUUGGAGUAAAAAUAA | 21 |
| 134 | SCN1A-IVS21−31 | GGAGUAAAAAUAAUUUAG | 21 |
| 135 | SCN1A-IVS21−36 | AAAAAUAAUUUAGACCUG | 21 |
| 136 | SCN1A-IVS21−41 | UAAUUUAGACCUGAUGUU | 21 |
| 137 | SCN1A-IVS21−46 | UAGACCUGAUGUUUAAUA | 21 |
| 138 | SCN1A-IVS21−51 | CUGAUGUUUAAUAAAUAU | 21 |
| 139 | SCN1A-IVS21−56 | GUUUAAUAAAUAUUCUUA | 21 |
| 140 | SCN1A-IVS21−61 | AUAAAUAUUCUUACUGAU | 21 |
| 141 | SCN1A-IVS21−66 | UAUUCUUACUGAUAUAAU | 21 |
| 142 | SCN1A-IVS21−71 | UUACUGAUAUAAUUUUCA | 21 |
| 143 | SCN1A-IVS21−76 | GAUAUAAUUUUCAAAAGG | 21 |
| 144 | SCN1A-IVS21−81 | AAUUUUCAAAAGGGAAUA | 21 |
| 145 | SCN1A-IVS21−27 | CUUUGGAGUAAAAAUAAU | 21 |
| 146 | SCN1A-IVS21−28 | UUUGGAGUAAAAAUAAUU | 21 |
| 148 | SCN1A-IVS21−29 | UUGGAGUAAAAAUAAUUU | 21 |
| 149 | SCN1A-IVS21−30 | UGGAGUAAAAAUAAUUUA | 21 |
| 150 | SCN1A-IVS21−32 | GAGUAAAAAUAAUUUAGA | 21 |
| 151 | SCN1A-IVS21−33 | AGUAAAAAUAAUUUAGAC | 21 |
| 152 | SCN1A-IVS21−34 | GUAAAAAUAAUUUAGACC | 21 |
| 153 | SCN1A-IVS21−35 | UAAAAAUAAUUUAGACCU | 21 |
| 154 | SCN1A-IVS21−72 | UACUGAUAUAAUUUUCAA | 21 |
| 155 | SCN1A-IVS21−73 | ACUGAUAUAAUUUUCAAA | 21 |
| 156 | SCN1A-IVS21−74 | CUGAUAUAAUUUUCAAAA | 21 |
| 157 | SCN1A-IVS21−75 | UGAUAUAAUUUUCAAAAG | 21 |
| 158 | SCN1A-IVS21−77 | AUAUAAUUUUCAAAAGGG | 21 |
| 159 | SCN1A-IVS21−78 | UAUAAUUUUCAAAAGGGA | 21 |
| 160 | SCN1A-IVS21−79 | AUAAUUUUCAAAAGGGAA | 21 |
| 161 | SCN1A-IVS21−80 | UAAUUUUCAAAAGGGAAU | 21 |
| 162 | | CAAGGAUUAAAGGUAGCA | 21 |

TABLE 1b
Exemplary ASOs to correct intron retention

| SEQ | | | Retained |
|---|---|---|---|
| ID NO: | Name | SeqTence (5′-3′) | Intron |
| 163 | SCN1A-IVS21+6 | CAGAGAAAATAGTGTTCA | 21 |
| 164 | SCN1A-IVS21+11 | ATATTCAGAGAAAATAGT | 21 |
| 165 | SCN1A-IVS21+16 | TAAAAATATTCAGAGAAA | 21 |
| 166 | SCN1A-IVS21+21 | AACAATAAAAATATTCAG | 21 |
| 167 | SCN1A-IVS21+26 | TTCCAAACAATAAAAATA | 21 |
| 168 | SCN1A-IVS21+31 | TATTATTCCAAACAATAA | 21 |
| 169 | SCN1A-IVS21+36 | TTTGTTATTATTCCAAAC | 21 |
| 170 | SCN1A-IVS21+41 | ATTATTTTGTTATTATTC | 21 |
| 171 | SCN1A-IVS21+46 | ATGTCATTATTTTGTTAT | 21 |
| 172 | SCN1A-IVS21+51 | GATGTATGTCATTATTTT | 21 |
| 173 | SCN1A-IVS21+56 | TAATAGATGTATGTCATT | 21 |
| 174 | SCN1A-IVS21+61 | CTAAATAATAGATGTATG | 21 |
| 175 | SCN1A-IVS21+66 | AGGAACTAAATAATAGAT | 21 |
| 176 | SCN1A-IVS21+71 | TTCTTAGGAACTAAATAA | 21 |
| 177 | SCN1A-IVS21+76 | ACTTTTTCTTAGGAACTA | 21 |
| 178 | SCN1A-IVS21+81 | TATATACTTTTTCTTAGG | 21 |
| 179 | SCN1A-IVS21−16 | TGCATGTTTTACTTTGGA | 21 |
| 180 | SCN1A-IVS21−21 | GTTTTACTTTGGAGTAAA | 21 |
| 181 | SCN1A-IVS21−26 | ACTTTGGAGTAAAAATAA | 21 |
| 182 | SCN1A-IVS21−31 | GGAGTAAAAATAATTTAG | 21 |
| 183 | SCN1A-IVS21−36 | AAAAATAATTTAGACCTG | 21 |
| 184 | SCN1A-IVS21−41 | TAATTTAGACCTGATGTT | 21 |
| 185 | SCN1A-IVS21−46 | TAGACCTGATGTTTAATA | 21 |
| 186 | SCN1A-IVS21−51 | CTGATGTTTAATAAATAT | 21 |
| 187 | SCN1A-IVS21−56 | GTTTAATAAATATTCTTA | 21 |
| 188 | SCN1A-IVS21−61 | ATAAATATTCTTACTGAT | 21 |
| 189 | SCN1A-IVS21−66 | TATTCTTACTGATATAAT | 21 |
| 190 | SCN1A-IVS21−71 | TTACTGATATAATTTTCA | 21 |
| 191 | SCN1A-IVS21−76 | GATATAATTTTCAAAAGG | 21 |
| 192 | SCN1A-IVS21−81 | AATTTTCAAAAGGGAATA | 21 |
| 193 | SCN1A-IVS21−27 | CTTTGGAGTAAAAATAAT | 21 |
| 194 | SCN1A-IVS21−28 | TTTGGAGTAAAAATAATT | 21 |
| 195 | SCN1A-IVS21−29 | TTGGAGTAAAAATAATTT | 21 |
| 196 | SCN1A-IVS21−30 | TGGAGTAAAAATAATTTA | 21 |
| 197 | SCN1A-IVS21−32 | GAGTAAAAATAATTTAGA | 21 |
| 198 | SCN1A-IVS21−33 | AGTAAAAATAATTTAGAC | 21 |
| 199 | SCN1A-IVS21−34 | GTAAAAATAATTTAGACC | 21 |
| 200 | SCN1A-IVS21−35 | TAAAAATAATTTAGACCT | 21 |
| 201 | SCN1A-IVS21−72 | TACTGATATAATTTTCAA | 21 |
| 202 | SCN1A-IVS21−73 | ACTGATATAATTTTCAAA | 21 |
| 203 | SCN1A-IVS21−74 | CTGATATAATTTTCAAAA | 21 |
| 204 | SCN1A-IVS21−75 | TGATATAATTTTCAAAAG | 21 |
| 205 | SCN1A-IVS21−77 | ATATAATTTTCAAAAGGG | 21 |
| 206 | SCN1A-IVS21−78 | TATAATTTTCAAAAGGGA | 21 |
| 207 | SCN1A-IVS21−79 | ATAATTTTCAAAAGGGAA | 21 |
| 208 | SCN1A-IVS21−80 | TAATTTTCAAAAGGGAAT | 21 |
| 209 | | CAAGGATTAAAGGTAGCA | 21 |

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present invention may vary depending on cell type to which delivery of the ASOs is desired. Multiple tissues and organs are affected by Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP, with the brain being the most significantly affected tissue. The ASOs of the present invention may be administered to patients parenterally, for example, by intrathecal injection, intracerebroventricular injection, intraperitoneal injection, intramuscular injection, subcutaneous injection, intravitreal injection, or intravenous injection.

In some embodiments, the disease or condition is induced by a mutation in Nay1.1 (a protein encoded by the SCN1A gene). In some instances, the mutation is a loss-of-function mutation in Nay1.1. In some cases, the loss-of-function mutation in Nay1.1 comprises one or more mutations that decreases or impairs the function of Nay1.1 (e.g., by 10%, 20%, 30%, 40%, 50%, 60%, 70%, 80%, 90%, 95%, or more) relative to the function of a wild-type Nay1.1. In some cases, the loss-of-function mutation in Nay1.1 comprises one or more mutations that result in a disease phenotype. Exemplary loss-of-function mutations include, but are not limited to, R859C, T875M, V1353L, I1656M, R1657C, A1685V, M1841T, and R1916G.

In other instances, the mutation is a gain-of-function mutation in Nay1.1. In such cases, the gain-of-function mutation comprises one or more mutations that prolongs activation of Nay1.1 (e.g., by 10%, 20%, 30%, 40%, 50%, 60%, 70%, 80%, 90%, 95%, or more) relative to the function of a wild-type Nay1.1. In such cases, the gain-of-function mutation in Nay1.1 comprises one or more mutations that result in a disease phenotype. Exemplary gain-of-function mutations include, but are not limited to, D188V, W1204R, R1648H, and D1866Y.

In some embodiments, the disease or condition is an encephalopathy. In some cases, the encephalopathy is induced by a loss-of-function mutation in Nay1.1.

In some embodiments, the encephalopathy is epileptic encephalopathy. Exemplary epileptic encephalopathies include, but are not limited to, Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); early infantile SCN1A encephalopathy; early infantile epileptic encephalopathy (EIEE); or sick sinus syndrome 1. In some embodiments, the disease or condition is epileptic encephalopathy, optionally selected from Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); and sick sinus syndrome 1.

In some instances, GEFS+ is epilepsy, generalized, with febrile seizures plus, type 2.

In some instances, the Febrile seizure is Febrile seizures, familial, 3A.

In some instances, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

In some embodiments, the diseases or conditions induced by a loss-of-function mutation in Nay1.1 include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; or malignant migrating partial seizures of infancy.

In some embodiments, the disease or condition is induced by a gain-of-function mutation in Nay1.1. Exemplary diseases or conditions associated with a gain-of-function mutation in Nay1.1 include, but are not limited to, migraine. In some instances, the disease or condition induced by a gain-of-function mutation in Nay1.1 is migraine.

In some instances, the migraine is migraine, familial hemiplegic, 3.

In some embodiments, the disease or condition is a Nay1.1 genetic epilepsy. The Nay1.1 genetic epilepsy can include a loss-of-function mutation in Nay1.1 or a gain-of-function mutation in Nay1.1. In some cases, the Nay1.1 genetic epilepsy includes one or more hereditary mutations.

In other cases, the Nay1.1 genetic epilepsy includes one or more de novo mutations. In some cases, the Nay1.1 genetic epilepsy includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); or malignant migrating partial seizures of infancy. In some cases, the Nay1.1 genetic epilepsy associated with a loss-of-function mutation in Nay1.1 includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); malignant migrating partial seizures of infancy.

In some embodiments, the disease or condition is associated with a haploinsufficiency of the SCN1A gene. Exemplary diseases or conditions associated with a haploinsufficiency of the SCN1A gene include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy. In some cases, the disease or condition is Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy.

In some cases, the disease or condition is Dravet Syndrome (DS).

Dravet syndrome (DS), otherwise known as severe myoclonic epilepsy of infancy (SMEI), is an epileptic encephalopathy presenting in the first year of life. Dravet syndrome is an increasingly recognized epileptic encephalopathy in which the clinical diagnosis is supported by the finding of sodium channel gene mutations in approximately 70-80% of patients. Mutations of ion channel genes play a major role in the pathogenesis of a range of epilepsy syndromes, resulting in some epilepsies being regarded as channelopathies. Voltage-gated sodium channels (VGSCs) play an essential role in neuronal excitability; therefore, it is not surprising that many mutations associated with DS have been identified in the gene encoding a VGSC subunit. The disease is described by, e.g., Mulley, et al., 2005, and the disease description at OMIM #607208 (Online Mendelian Inheritance in Man, Johns Hopkins University, 1966-2015), both incorporated by reference herein.

Between 70% and 80% of patients carry sodium channel al subunit gene (SCN1A) abnormalities, and truncating mutations account for about 40%, and have a significant correlation with an earlier age of seizures onset. Sequencing mutations are found in about 70% of cases and comprise truncating (40%) and missense mutations (40%) with the remaining being splice-site changes. Most mutations are de novo, but familial mutations occur in 5-10% of cases and are usually missense in nature. The remaining SCN1A mutations comprise splice-site and missense mutations, most of which fall into the pore-forming region of the sodium channel. At present, over 500 mutations have been associated with DS and are randomly distributed along the gene (Mulley, et al., Neurol. 2006, 67, 1094-1095).

The SCN1A gene is located in the cluster of sodium channel genes on human chromosome 2q24 and encodes the α-pore forming subunits known as Nav1.1 of the neuronal voltage gated sodium channel. The SCN1A gene spans approximately 100 kb of genomic DNA and comprises 26 exons. The SCN1A protein consists of four domains, each with six-transmembrane segments. Two splice variants have been identified that result in a long and short isoform that differ in the presence or absence of 11 amino acids in the cytoplasmic loop between domains 1 and 2, in exon 11 (Miller, et al., 1993-2015, and Mulley, et al., 2005, 25, 535-542, incorporated herein by reference).

Alternative splicing events in SCN1A gene can lead to non-productive mRNA transcripts which in turn can lead to aberrant protein expression, and therapeutic agents which can target the alternative splicing events in SCN1A gene can modulate the expression level of functional proteins in DS patients and/or inhibit aberrant protein expression. Such therapeutic agents can be used to treat a condition caused by SCN1A protein deficiency.

One of the alternative splicing events that can lead to non-productive mRNA transcripts is the inclusion of an extra exon in the mRNA transcript that can induce non-sense mediated mRNA decay. The present disclosure provides compositions and methods for modulating alternative splicing of SCN1A to increase the production of protein-coding mature mRNA, and thus, translated functional SCN1A protein. These compositions and methods include antisense oligomers (ASOs) that can cause exon skipping and promote constitutive splicing of SCN1A pre-mRNA. In various embodiments, functional SCN1A protein can be increased using the methods of the disclosure to treat a condition caused by SCN1A protein deficiency.

In some cases, the disease or condition is SMEB.

In some cases, the disease or condition is GEFS+.

In some cases, the disease or condition is a Febrile seizure (e.g., Febrile seizures, familial, 3A).

In some cases, the disease or condition is autism (also known as autism spectrum disorder or ASD).

In some cases, the disease or condition is migraine (e.g., migraine, familial hemiplegic, 3).

In some cases, the disease or condition is Alzheimer's disease.

In some embodiments, the disease or condition is SCN2A encephalopathy.

In some embodiments, the disease or condition is SCN8A encephalopathy.

In some embodiments, the disease or condition is SCN5A arrhythmia.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In embodiments, an ASO of the invention is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In embodiments, subjects treated using the methods and compositions are evaluated for improvement in condition using any methods known and described in the art.

Methods of Identifying Additional ASOs that Induce Exon Skipping

Also within the scope of the present disclosure are methods for identifying or determining ASOs that induce exon skipping of a SCN1A NIE containing pre-mRNA. For example, a method can comprise identifying or determining ASOs that induce pseudo-exon skipping of a SCN1A NIE containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., pseudo-exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the included exon (e.g., a portion of sequence of the exon located upstream of the target/included exon) to approximately 100 nucleotides downstream of the 3′ splice site of the target/included exon and/or from approximately 100 nucleotides upstream of the 5′ splice site of the included exon to approximately 100 nucleotides downstream of the 5′ splice site of the target/included exon (e.g., a portion of sequence of the exon located downstream of the target/included exon). For example, a first ASO of 15 nucleotides in length may be designed to specifically hybridize to nucleotides+6 to +20 relative to the 3′ splice site of the target/included exon. A second ASO may be designed to specifically hybridize to nucleotides+11 to +25 relative to the 3′ splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NIE containing pre-mRNA described herein). The exon skipping effects of each of the ASOs may be assessed by any method known in the art, for example by reverse transcriptase (RT)-PCR using primers that span the splice junction, as described in Example 4. A reduction or absence of a longer RT-PCR product produced using the primers spanning the region containing the included exon (e.g. including the flanking exons of the NIE) in ASO-treated cells as compared to in control ASO-treated cells indicates that splicing of the target NIE has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NIE).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NIE, as described herein (see, e.g., Example 4). A reduction or absence of a longer RT-PCR product produced using the primers spanning the NIE in ASO-treated cells as compared to in control ASO-treated cells indicates that exon skipping (or splicing of the target intron containing an NIE) has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NIE) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, intravitreal injection, or intravenous injection. Following administration, the cells, tissues, and/or organs of the model animals may be assessed to determine the effect of the ASO treatment by for example evaluating splicing (efficiency, rate, extent) and protein production by methods known in the art and described herein. The animal models may also be any phenotypic or behavioral indication of the disease or disease severity.

As described herein in various examples, exon 20× in human SCN1A gene is equivalent to exon 21× in mouse SCN1A gene.

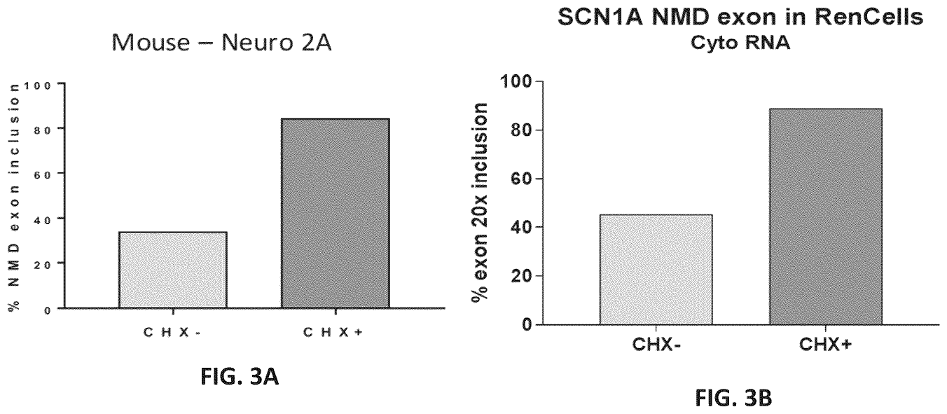

Also within the scope of the present disclosure is a method to identify or validate an NMD-inducing exon in the presence of an NMD inhibitor, for example, cycloheximide. An exemplary method is provided in FIG. 3 and Example 2.

### Specific Embodiments

Embodiment 1. A method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell.

Embodiment 2. A method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject.

Embodiment 3. The method of embodiment 1 or 2, wherein the therapeutic agent
(a) binds to a targeted portion of the NMD exon mRNA encoding SCN1A;
(b) modulates binding of a factor involved in splicing of the NMD exon mRNA; or
(c) a combination of (a) and (b).

Embodiment 4. The method of embodiment 3, wherein the therapeutic agent interferes with binding of the factor involved in splicing of the NMD exon from a region of the targeted portion.

Embodiment 5. The method of embodiment 3 or 4, wherein the targeted portion is proximal to the NMD exon.

Embodiment 6. The method of any one of embodiments 3 to 5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

Embodiment 7. The method of any one of embodiments 3 to 6, wherein the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

Embodiment 8. The method of any one of embodiments 3 to 5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

Embodiment 9. The method of any one of embodiments 3 to 5 or 8, wherein the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

Embodiment 10. The method of any one of embodiments 3 to 9, wherein the targeted portion is located in an intronic region between two canonical exonic regions of the NMD exon mRNA encoding SCN1A, and wherein the intronic region contains the NMD exon.

Embodiment 11. The method of any one of embodiments 3 to 10, wherein the targeted portion at least partially overlaps with the NMD exon.

Embodiment 12. The method of any one of embodiments 3 to 11, wherein the targeted portion at least partially overlaps with an intron upstream of the NMD exon.

Embodiment 13. The method of any one of embodiments 3 to 12, wherein the targeted portion comprises 5′ NMD exon-intron junction or 3′ NMD exon-intron junction.

Embodiment 14. The method of any one of embodiments 3 to 13, wherein the targeted portion is within the NMD exon.

Embodiment 15. The method of any one of embodiments 3 to 14, wherein the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

Embodiment 16. The method of any one of embodiments 1 to 15, wherein the NMD exon mRNA encoding SCN1A comprises a sequence with at least about 80%, 85%, 90%, 95%, 97%, or 100% sequence identity to any one of SEQ ID NOs: 2 or 7-10.

Embodiment 17. The method of any one of embodiments 1 to 16, wherein the NMD exon mRNA encoding SCN1A is encoded by a genetic sequence with at least about 80%, 85%, 90%, 95%, 97%, or 100% sequence identity to SEQ ID NOs: 1 or 3-6.

Embodiment 18. The method of any one of embodiments 3 to 17, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh37/hg19: chr2:166,863,803.

Embodiment 19. The method of any one of embodiments 3 to 18, wherein the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of genomic site GRCh37/hg19: chr2:166,863,803.

Embodiment 20. The method of any one of embodiments 3 to 17, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh37/hg19: chr2:166,863,740.

Embodiment 21. The method of any one of embodiments 3 to 17 or 20, wherein the targeted portion is about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of genomic site GRCh37/hg19: chr2:166,863,740.

Embodiment 22. The method of any one of embodiments 3 to 21, wherein the targeted portion of the NMD exon mRNA encoding SCN1A comprises a sequence with at least 80%, 85%, 90%, 95%, 97%, or 100% sequence identity to a region comprising at least 8 contiguous nucleic acids of SEQ ID NO: SEQ ID NOs: 2 or 7-10.

Embodiment 23. The method of embodiment 22, wherein the therapeutic agent is an antisense oligomer (ASO) and wherein the ASO comprises a sequence that is at least about 80%, 85%, 90%, 95%, 97%, or 100% identity to any one of SEQ ID NOs: 21-67, 210-256, or 304-379.

Embodiment 24. The method of any one of embodiments 3 to 21, wherein the targeted portion of the NMD exon mRNA encoding SCN1A is within the non-sense mediated RNA decay-inducing exon 20× of SCN1A.

Embodiment 25. The method of embodiment 24, wherein the therapeutic agent is an antisense oligomer (ASO) and wherein the ASO comprises a sequence that is at least about 80%, 85%, 90%, 95%, 97%, or 100% identity to any one of SEQ ID NOs: 42-50, or 231-239.

Embodiment 26. The method of any one of embodiments 3 to 21, wherein the targeted portion of the NMD exon mRNA encoding SCN1A is upstream or downstream of the non-sense mediated RNA decay-inducing exon 20× of SCN1A.

Embodiment 27. The method of embodiment 26, wherein the therapeutic agent is an antisense oligomer (ASO) and wherein the ASO comprises a sequence that is at least about 80%, 85%, 90%, 95%, 97%, or 100% identity to any one of SEQ ID NOs: 21-38, 53-67, 210-227, or 242-256.

Embodiment 28. The method of any one of embodiments 3 to 21, wherein the targeted portion of the NMD exon mRNA comprises an exon-intron junction of exon 20× of SCN1A.

Embodiment 29. The method of embodiment 28, wherein the therapeutic agent is an antisense oligomer (ASO) and wherein the ASO comprises a sequence that is at least about 80%, 85%, 90%, 95%, 97%, or 100% identity to any one of SEQ ID NOs: 39-41, 51, 52, 228-230, 240, or 241.

Embodiment 30. The method of any one of embodiments 1 to 29, wherein the therapeutic agent promotes exclusion of the NMD exon from the processed mRNA encoding SCN1A protein.

Embodiment 31. The method of embodiment 30, wherein exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell.

Embodiment 32. The method of embodiment 30 or 31, wherein the therapeutic agent increases level of the processed mRNA encoding SCN1A protein in the cell.

Embodiment 33. The method of any one of embodiments 30 to 32, wherein an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell.

Embodiment 34. The method of any one of embodiments 30 to 33, wherein the therapeutic agent increases expression of SCN1A protein in the cell.

Embodiment 35. The method of any one of embodiments 30 to 34, wherein an amount of SCN1A produced in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell.

Embodiment 36. The method of any one of embodiments 2 to 35, wherein the disease or condition is induced by a loss-of-function mutation in Nav1.1.

Embodiment 37. The method of any one of embodiments 2 to 36, wherein the disease or condition is associated with haploinsufficiency of the SCN1A gene, and wherein the subject has a first allele encoding a functional SCN1A, and a second allele from which SCN1A is not produced or produced at a reduced level, or a second allele encoding a nonfunctional SCN1A or a partially functional SCN1A.

Embodiment 38. The method of any one of embodiments 2 to 37, wherein the disease or condition is encephalopathy.

Embodiment 39. The method of embodiment 38, wherein the encephalopathy is epileptic encephalopathy.

Embodiment 40. The method of any one of embodiments 2 to 37, wherein the disease or condition is Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or malignant migrating partial seizures of infancy.

Embodiment 41. The method of embodiment 40, wherein GEFS+ is epilepsy, generalized, with febrile seizures plus, type 2.

Embodiment 42. The method of embodiment 40, wherein the Febrile seizure is Febrile seizures, familial, 3A.

Embodiment 43. The method of embodiment 40, wherein SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

Embodiment 44. The method of any one of embodiments 1 to 43, wherein the therapeutic agent promotes exclusion of the NMD exon from the processed mRNA encoding SCN1A protein and increases the expression of SCN1A in the cell.

Embodiment 45. The method of any one of embodiments 1 to 44, wherein the therapeutic agent is an antisense oligomer (ASO) and wherein the ASO comprises a sequence that is at least about 80%, 85%, 90%, 95%, 97%, or 100% complimentary to any one of SEQ ID NOs: 22-24, 26, 27, 29-35, 37-62, 64-67, or 304-379.

Embodiment 46. The method of any one of embodiments 1 to 29, wherein the therapeutic agent inhibits exclusion of the NMD exon from the processed mRNA encoding SCN1A protein.

Embodiment 47. The method of embodiment 46, wherein exclusion of the NMD exon from the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell.

Embodiment 48. The method of embodiment 46 or 47, wherein the therapeutic agent decreases level of the processed mRNA encoding SCN1A protein in the cell.

Embodiment 49. The method of any one of embodiments 46 to 48, wherein an amount of the processed mRNA encoding SCN1A protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell.

Embodiment 50. The method of any one of embodiments 46 to 49, wherein the therapeutic agent decreases expression of SCN1A protein in the cell.

Embodiment 51. The method of any one of embodiments 46 to 50, wherein an amount of SCN1A produced in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of SCN1A produced in a control cell.

Embodiment 52. The method of any one of embodiments 2 to 29 or 46 to 49, wherein the disease or condition is induced by a gain-of-function mutation in Nav1.1.

Embodiment 53. The method of embodiment 52, wherein the subject has an allele from which SCN1A is produced at an increased level, or an allele encoding a mutant SCN1A that induces increased activity of Nav1.1 in the cell.

Embodiment 54. The method of embodiment 52 or 53, wherein the disease or condition is migraine.

Embodiment 55. The method of embodiment 54, wherein the migraine is migraine, familial hemiplegic, 3.

Embodiment 56. The method of any one of embodiments 2 to 49, wherein the disease or condition is a Nav1.1 genetic epilepsy.

Embodiment 57. The method of any one of embodiments 46 to 56, wherein the therapeutic agent inhibits exclusion of the NMD exon from the processed mRNA encoding SCN1A protein and decreases the expression of SCN1A in the cell.

Embodiment 58. The method of any one of embodiments 46 to 57, wherein the therapeutic agent is an antisense oligomer (ASO) and wherein the ASO comprises a sequence that is at least about 80%, 85%, 90%, 95%, 97%, or 100% complimentary to any one of SEQ ID NOs: 21, 25, 28, 36, or 63.

Embodiment 59. The method of any one of previous embodiments, wherein the therapeutic agent is an antisense oligomer (ASO) and wherein the antisense oligomer comprises a backbone modification comprising a phosphorothioate linkage or a phosphorodiamidate linkage.

Embodiment 60. The method of any one of previous embodiments, wherein the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment 61. The method of any one of previous embodiments, wherein the therapeutic agent is an antisense oligomer (ASO) and wherein the antisense oligomer comprises at least one modified sugar moiety.

Embodiment 62. The method of embodiment 61, wherein each sugar moiety is a modified sugar moiety.

Embodiment 63. The method of any one of previous embodiments, wherein the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment 64. The method of any one of embodiments 3 to 63, wherein the therapeutic agent is an antisense oligomer (ASO) and wherein the antisense oligomer is at least 80%, at least 85%, at least 90%, at least 95%, at least 98%, at least 99%, or 100%, complementary to the targeted portion of the NMD exon mRNA encoding the protein.

Embodiment 65. The method of any one of previous embodiments, wherein the method further comprises assessing SCN1A mRNA or protein expression.

Embodiment 66. The method of any one of embodiments 2 to 65, wherein the subject is a human.

Embodiment 67. The method of any one of embodiments 2 to 65, wherein the subject is a non-human animal.

Embodiment 68. The method of any one of embodiments 2 to 65, wherein the subject is a fetus, an embryo, or a child.

Embodiment 69. The method of any one of previous embodiments, wherein the cells are ex vivo.

Embodiment 70. The method of any one of embodiments 2 to 69, wherein the therapeutic agent is administered by intrathecal injection, intracerebroventricular injection, intraperitoneal injection, intramuscular injection, subcutaneous injection, intravitreal, or intravenous injection of the subject.

Embodiment 71. The method of any one of embodiments 2 to 65, wherein the method further comprises administering a second therapeutic agent to the subject.

Embodiment 72. The method of embodiment 71, wherein the second therapeutic agent is a small molecule.

Embodiment 73. The method of embodiment 71, wherein the second therapeutic agent is an ASO.

Embodiment 74. The method of embodiment 73, wherein the ASO comprises a sequence that is at least about 80%, 85%, 90%, 95%, 97%, or 100% complimentary to any one of SEQ ID NOs: 115-161.

Embodiment 75. The method of embodiment 71, wherein the second therapeutic agent corrects intron retention.

Embodiment 76. The method of any one of embodiments 2 to 65, wherein the disease or condition is Alzheimer's Disease, SCN2A encephalopathy, SCN8A encephalopathy, or SCN5A arrythmia.

Embodiment 77. The method of embodiment 30, 32 or 34, wherein the disease or condition is Alzheimer's Disease, SCN2A encephalopathy, SCN8A encephalopathy, or SCN5A arrythmia.

Embodiment 78. A method of treating Dravet Syndrome (DS); Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or sudden unexpected death in epilepsy (SUDEP) in a subject in need thereof, by increasing the expression of a target protein or functional RNA by a cell of the subject, wherein the cell has an mRNA that contains a non-sense mediated RNA decay-inducing exon (NMD exon mRNA), and wherein the NMD exon mRNA encodes the target protein or functional RNA, the method comprising contacting the cell of the subject with a therapeutic agent that binds to a targeted portion of the NMD exon mRNA encoding the target protein or functional RNA, whereby the non-sense mediated RNA decay-inducing exon is excluded from the NMD exon mRNA encoding the target protein or functional RNA, thereby increasing the level of processed mRNA encoding the target protein or functional RNA, and increasing the expression of the target protein or functional RNA in the cell of the subject.

Embodiment 79. The method of embodiment 78, wherein the target protein is SCN1A.

Embodiment 80. A method of increasing expression of SCN1A protein by a cell having an mRNA that contains a non-sense mediated RNA decay-inducing exon (NMD exon mRNA) and encodes SCN1A protein, the method comprising contacting the cell an agent that binds to a targeted portion of the NMD exon mRNA encoding SCN1A protein, whereby the non-sense mediated RNA decay-inducing exon is excluded from the NMD exon mRNA encoding SCN1A protein, thereby increasing the level of processed mRNA encoding SCN1A protein, and increasing the expression of SCN1A protein in the cell.

Embodiment 81. A method of treating a disease or condition in a subject in need thereof by increasing the expression of SCN1A protein in a cell of the subject, comprising: contacting the cell of the subject with a therapeutic agent that binds to a targeted portion of a non-sense mediated RNA decay-inducing exon mRNA encoding the SCN1A protein or functional SCN1A RNA, whereby the non-sense mediated RNA decay-inducing exon is excluded from the NMD exon mRNA encoding the SCN1A protein or functional SCN1A RNA, thereby increasing the level of processed mRNA encoding the SCN1A protein or functional SCN1A RNA, and increasing the expression of the SCN1A protein or functional SCN1A RNA in the cell of the subject; wherein the disease or condition is associated with a mutation of a gene other than an SCN1A gene, aberrant expression of a protein encoded by a gene other than an SCN1A gene or aberrant expression of an RNA encoded by a gene other than an SCN1A gene.

Embodiment 82. The method of embodiment 81, wherein a symptom of the disease or condition is reduced by about 2-fold, 3-fold, 4-fold, 5-fold, 6-fold, 7-fold, 8-fold, 9-fold, 10-fold, or more.

Embodiment 83. The method of embodiment 81 or 82, wherein a symptom of the disease or condition is reduced by about 20%, 30%, 40%, 50%, 60%, 70%, 80%, 90%, 95%, or 100% with an increase in expression of the SCN1A protein.

Embodiment 84. The method of any one of embodiments 81 to 83, wherein progression of the disease or condition is reduced by about 2-fold, 3-fold, 4-fold, 5-fold, 6-fold, 7-fold, 8-fold, 9-fold, 10-fold, or more with an increase in expression of the SCN1A protein.

Embodiment 85. The method of any one of embodiments 81 to 84, wherein progression of the disease or condition is reduced by about 20%, 30%, 40%, 50%, 60%, 70%, 80%, 90%, 95%, or 100% with an increase in expression of the SCN1A protein.

Embodiment 86. The method of any one of embodiments 81 to 85, wherein increasing the expression of the SCN1A protein or functional SCN1A RNA compensates for the mutation of a gene other than an SCN1A gene, the aberrant expression of a protein encoded by a gene other than an SCN1A gene or the aberrant expression of an RNA encoded by a gene other than an SCN1A gene.

Embodiment 87. The method of any one of embodiments 81 to 86, wherein the disease or condition is epileptic encephalophathy, early infantile, 13.

Embodiment 88. The method of any one of embodiments 81 to 87, wherein the subject has a mutation in the SCN8A gene.

Embodiment 89. The method of any one of embodiments 81 to 86, wherein the disease or condition is sick sinus syndrome 1.

Embodiment 90. The method of any one of embodiments 81 to 86 or 88, wherein the subject has a mutation in the SCN5A gene

Embodiment 91. The method of any one of embodiments 81 to 86, wherein the disease or condition is Alzheimer's disease.

Embodiment 92. A method of treating a disease or condition in a subject in need thereof, comprising administering to the subject a composition comprising an antisense oligomer, the antisense oligomer comprising a sequence of at least 8 contiguous nucleotides that is at least 80%, 85%, 90%, 95%, 97%, or 100% complementary to intron 20 of SCN1A.

Embodiment 93. A method of treating a disease or condition in a subject in need thereof, comprising administering to the subject a composition comprising an antisense oligomer, the antisense oligomer comprising a sequence of at least 8 contiguous nucleotides that is at least 80%, 85%, 90%, 95%, 97%, or 100% complementary to any one of SEQ ID NOs: 7-10.

Embodiment 94. The method of any one of embodiments 78 to 93, wherein the non-sense mediated RNA decay-inducing exon is spliced out from the NMD exon mRNA encoding the target protein or functional RNA.

Embodiment 95. The method of any one of embodiments 78 to 94, wherein the target protein does not comprise an amino acid sequence encoded by the non-sense mediated RNA decay-inducing exon.

Embodiment 96. The method of any one of embodiments 78 to 95, wherein the target protein is a full-length target protein.

Embodiment 97. The method of any one of embodiments 78 to 96, wherein the agent is an antisense oligomer (ASO) complementary to the targeted portion of the NMD exon mRNA.

Embodiment 98. The method of any one of embodiments 78 to 97, wherein the mRNA is pre-mRNA.

Embodiment 99. The method of any one of embodiments 78 to 98, wherein the contacting comprises contacting the therapeutic agent to the mRNA, wherein the mRNA is in a nucleus of the cell.

Embodiment 100. The method of any one of embodiments 78 to 99, wherein the target protein or the functional RNA corrects a deficiency in the target protein or functional RNA in the subject.

Embodiment 101. The method of any one of embodiments 78 to 100, wherein the cells are in or from a subject with a condition caused by a deficient amount or activity of SCN1A protein.

Embodiment 102. The method of any one of embodiments 78 to 101, wherein the deficient amount of the target protein is caused by haploinsufficiency of the target protein, wherein the subject has a first allele encoding a functional target protein, and a second allele from which the target protein is not produced or produced at a reduced level, or a second allele encoding a nonfunctional or partially functional target protein, and wherein the antisense oligomer binds to a targeted portion of a NMD exon mRNA transcribed from the first allele.

Embodiment 103. The method of any one of embodiments 78 to 101, wherein the subject has a condition caused by a disorder resulting from a deficiency in the amount or function of the target protein, wherein the subject has
(a) a first mutant allele from which
   (i) the target protein is produced at a reduced level compared to production from a wild-type allele,
   (ii) the target protein is produced in a form having reduced function compared to an equivalent wild-type protein, or
   (iii) the target protein is not produced, and
(b) a second mutant allele from which
   (i) the target protein is produced at a reduced level compared to production from a wild-type allele,
   (ii) the target protein is produced in a form having reduced function compared to an equivalent wild-type protein, or
   (iii) the target protein is not produced, and wherein when the subject has a first mutant allele (a)(iii), the second mutant allele is (b)(i) or (b)(ii) and wherein when the subject has a second mutant allele (b)(iii), the first mutant allele is (a)(i) or (a)(ii), and wherein the NMD exon mRNA is transcribed from either the first mutant allele that is (a)(i) or (a)(ii), and/or the second allele that is (b)(i) or (b)(ii).

Embodiment 104. The method of embodiment 103, wherein the target protein is produced in a form having reduced function compared to the equivalent wild-type protein.

Embodiment 105. The method of embodiment 103, wherein the target protein is produced in a form that is fully-functional compared to the equivalent wild-type protein.

Embodiment 106. The method of any one of embodiments 78 to 105, wherein the targeted portion of the NMD exon mRNA is within the non-sense mediated RNA decay-inducing exon.

Embodiment 107. The method of any one of embodiments 78 to 105, wherein the targeted portion of the NMD exon mRNA is either upstream or downstream of the non-sense mediated RNA decay-inducing exon.

Embodiment 108. The method of any one of embodiments 78 to 107, wherein the NMD exon mRNA comprises a sequence with at least about 80%, 85%, 90%, 95%, 97%, or 100% sequence identity to any one of SEQ ID NOs: 2, 7-10, 12, and 17-20.

Embodiment 109. The method of any one of embodiments 78 to 107, wherein the NMD exon mRNA is encoded by a genetic sequence with at least about 80%, 85%, 90%, 95%, 97%, or 100% sequence identity to SEQ ID NOs: 1, 3-6, 11, and 13-16.

Embodiment 110. The method of any one of embodiments 78 to 107, wherein the targeted portion of the NMD exon mRNA comprises a sequence with at least 80%, 85%, 90%, 95%, 97%, or 100% sequence identity to a region comprising at least 8 contiguous nucleic acids of SEQ ID NO: SEQ ID NOs: 2, 7-10, 12, and 17-20.

Embodiment 111. The method of any one of embodiments 78 to 110, wherein the agent is an antisense oligomer (ASO) and wherein the ASO comprises a sequence that is at least about 80%, 85%, 90%, 95%, 97%, or 100% identity to any one of SEQ ID NOs: 21-114.

Embodiment 112. The method of any one of embodiments 78 to 105, wherein the targeted portion of the NMD exon mRNA is within the non-sense mediated RNA decay-inducing exon 20× of SCN1A.

Embodiment 113. The method of embodiment 112, wherein the agent is an antisense oligomer (ASO) and wherein the ASO comprises a sequence that is at least about 80%, 85%, 90%, 95%, 97%, or 100% identity to any one of SEQ ID NOs: 42-50, or 231-239.

Embodiment 114. The method of embodiment any one of embodiments 78 to 105, wherein the targeted portion of the NMD exon mRNA is upstream or downstream of the non-sense mediated RNA decay-inducing exon 20× of SCN1A.

Embodiment 115. The method of embodiment 114, wherein the agent is an antisense oligomer (ASO) and wherein the ASO comprises a sequence that is at least about 80%, 85%, 90%, 95%, 97%, or 100% identity to any one of SEQ ID NOs: 21-38, 53-67, 210-227, or 242-256.

Embodiment 116. The method of any one of embodiments 78 to 105, wherein the targeted portion of the NMD exon mRNA comprises an exon-intron junction of exon 20× of SCN1A.

Embodiment 117. The method of embodiment 116, wherein the agent is an antisense oligomer (ASO) and wherein the ASO comprises a sequence that is at least about 80%, 85%, 90%, 95%, 97%, or 100% identity to any one of SEQ ID NOs: 39-41, 51, 52, 228-230, 240, or 241.

Embodiment 118. The method of any one of embodiments 78 to 105, wherein the targeted portion of the NMD exon mRNA is within the non-sense mediated RNA decay-inducing exon 21× of Scn1a.

Embodiment 119. The method of embodiment 118, wherein the agent is an antisense oligomer (ASO) and wherein the ASO comprises a sequence that is at least about 80%, 85%, 90%, 95%, 97%, or 100% identity to any one of SEQ ID NOs: 89-97.

Embodiment 120. The method of embodiment any one of embodiments 78 to 105, wherein the targeted portion of the NMD exon mRNA is either upstream or downstream of the non-sense mediated RNA decay-inducing exon 21× of Scn1a.

Embodiment 121. The method of embodiment 120, wherein the agent is an antisense oligomer (ASO) and wherein the ASO comprises a sequence that is at least about 80%, 85%, 90%, 95%, 97%, or 100% identity to any one of SEQ ID NOs: 68-85 and 100-114.

Embodiment 122. The method of any one of embodiments 78 to 105, wherein the targeted portion of the NMD exon mRNA comprises an exon-intron junction of exon 21× of Scn1a.

Embodiment 123. The method of embodiment 122, wherein the agent is an antisense oligomer (ASO) and wherein the ASO comprises a sequence that is at least about 80%, 85%, 90%, 95%, 97%, or 100% identity to any one of SEQ ID NOs: 86-88 and 98-99.

Embodiment 124. The method of any one of embodiments 78 to 123, wherein the target protein produced is full-length protein, or wild-type protein.

Embodiment 125. The method of any one of embodiments 78 to 124, wherein the total amount of the processed mRNA encoding the target protein or functional RNA produced in the cell contacted with the antisense oligomer is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of the processed mRNA encoding the target protein or functional RNA produced in a control cell.

Embodiment 126. The method of one any of embodiments 78 to 124, wherein the total amount of target protein produced by the cell contacted with the antisense oligomer is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of target protein produced by a control cell.

Embodiment 127. The method of any one of embodiments 78 to 126, wherein the agent is an antisense oligomer (ASO) and wherein the antisense oligomer comprises a backbone modification comprising a phosphorothioate linkage or a phosphorodiamidate linkage.

Embodiment 128. The method of any one of embodiments 78 to 127, wherein the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment 129. The method of any one of embodiments 78 to 128, wherein the agent is an antisense oligomer (ASO) and wherein the antisense oligomer comprises at least one modified sugar moiety.

Embodiment 130. The method of embodiment 129, wherein each sugar moiety is a modified sugar moiety.

Embodiment 131. The method of any one of embodiments 78 to 130, wherein the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment 132. The method of any one of embodiments 78 to 131, wherein the agent is an antisense oligomer (ASO) and wherein the antisense oligomer is at least 80%, at least 85%, at least 90%, at least 95%, at least 98%, at least 99%, or 100%, complementary to the targeted portion of the NMD exon mRNA encoding the protein.

Embodiment 133. The method of any one of embodiments 78 to 132, wherein the method further comprises assessing SCN1A mRNA or protein expression.

Embodiment 134. The method of any one of embodiments 1 to 133, wherein Dravet Syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or sudden unexpected death in epilepsy (SUDEP) is treated and wherein the antisense oligomer binds to a targeted portion of a SCN1A NMD exon mRNA, wherein the targeted portion is within a sequence selected from SEQ ID NOs: 7-10 and 17-20.

Embodiment 135. The method of any one of embodiments 78 to 134, wherein the subject is a human.

Embodiment 136. The method of any one of embodiments 78 to 135, wherein the subject is a non-human animal.

Embodiment 137. The method of any one of embodiments 78 to 136, wherein the subject is a fetus, an embryo, or a child.

Embodiment 138. The method of any one of embodiments 78 to 137, wherein the cells are ex vivo.

Embodiment 139. The method of any one of embodiments 78 to 138, wherein the therapeutic agent is administered by intrathecal injection, intracerebroventricular injection, intraperitoneal injection, intramuscular injection, subcutaneous injection, intravitreal injection, or intravenous injection of the subject.

Embodiment 140. The method of any of embodiments 78 to 139, wherein the method further comprises administering a second therapeutic agent to the subject.

Embodiment 141. The method of embodiment 140, wherein the second therapeutic agent is a small molecule.

Embodiment 142. The method of embodiment 140, wherein the second therapeutic agent is an ASO.

Embodiment 143. The method of embodiment 142, wherein the ASO comprises a sequence that is at least about 80%, 85%, 90%, 95%, 97%, or 100% identity to any one of SEQ ID NOs: 115-161.

Embodiment 144. The method of any one of embodiments 140 to 142, wherein the second therapeutic agent corrects intron retention.

Embodiment 145. An antisense oligomer as used in a method of any of embodiments 78 to 144.

Embodiment 146. An antisense oligomer comprising a sequence with at least about 80%, 85%, 90%, 95%, 97%, or 100% sequence identity to any one of SEQ ID NOs: 21-114.

Embodiment 147. A pharmaceutical composition comprising the antisense oligomer of embodiment 145 or 146 and an excipient.

Embodiment 148. A method of treating a subject in need thereof, comprising administering the pharmaceutical composition of embodiment 147 to the subject, wherein the administering is by intrathecal injection, intracerebroventricular injection, intraperitoneal injection, intramuscular injection, subcutaneous injection, intravitreal injection, or intravenous injection.

Embodiment 149. A composition comprising a therapeutic agent for use in a method of increasing expression of a target protein or a functional RNA by cells to treat a disease or condition associated with a deficient protein or deficient functional RNA in a subject in need thereof, wherein the deficient protein or deficient functional RNA is deficient in amount or activity in the subject, wherein the target protein is:
(a) the deficient protein; or
(b) a compensating protein which functionally augments or replaces the deficient protein or in the subject;
and wherein the functional RNA is:
(c) the deficient RNA; or
(d) a compensating functional RNA which functionally augments or replaces the deficient functional RNA in the subject;

wherein the therapeutic agent enhances exclusion of the non-sense mediated RNA decay-inducing exon from the NMD exon mRNA encoding the target protein or functional RNA, thereby increasing production or activity of the target protein or the functional RNA in the subject.

Embodiment 150. A composition comprising a therapeutic agent for use in a method of treating a disease or condition in a subject in need thereof, the method comprising the step of modulating expression of SCN1A protein by cells of the subject, wherein the cells have an mRNA that contains a non-sense mediated RNA decay-inducing exon (NMD exon mRNA) and encodes SCN1A protein, the method comprising contacting the cells with the therapeutic agent, whereby exclusion of the non-sense mediated RNA decay-inducing exon from the NMD exon mRNA that encodes SCN1A protein is modulated, thereby modulating the level of processed mRNA encoding SCN1A protein, and modulating the expression of SCN1A protein in the cells of the subject.

Embodiment 151. The composition of embodiment 150, wherein the disease or condition is selected from the group consisting of: Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or migraine, familial hemiplegic, 3; and Alzheimer's Diseases.

Embodiment 152. The composition of any one of embodiments 150 to 151, wherein the SCN1A protein and NMD exon mRNA are encoded by the SCN1A gene.

Embodiment 153. The composition of any one of embodiments 149 to 152, wherein the non-sense mediated RNA decay-inducing exon is spliced out from the NMD exon mRNA encoding the SCN1A protein.

Embodiment 154. The composition of any one of embodiments 149 to 153, wherein the SCN1A protein does not comprise an amino acid sequence encoded by the non-sense mediated RNA decay-inducing exon.

Embodiment 155. The composition of any one of embodiments 149 to 154, wherein the SCN1A protein is a full-length SCN1A protein.

Embodiment 156. The composition of any one of embodiments 149 to 155, wherein the therapeutic agent is an antisense oligomer (ASO) complementary to the targeted portion of the NMD exon mRNA.

Embodiment 157. The composition of any of embodiments 149 to 156, wherein the therapeutic agent is an antisense oligomer (ASO) and wherein the antisense oligomer targets a portion of the NMD exon mRNA that is within the non-sense mediated RNA decay-inducing exon.

Embodiment 158. The composition of any of embodiments 149 to 156, wherein the therapeutic agent is an antisense oligomer (ASO) and wherein the antisense oligomer targets a portion of the NMD exon mRNA that is upstream or downstream of the non-sense mediated RNA decay-inducing exon.

Embodiment 159. The composition of any one of embodiments 149 to 158, wherein the target protein is SCN1A.

Embodiment 160. The composition of embodiment 159, wherein the NMD exon mRNA comprises a sequence with at least about 80%, 85%, 90%, 95%, 97%, or 100% sequence identity to any one of SEQ ID NOs: 2, 7-10, 12, and 17-20.

Embodiment 161. The composition of embodiment 159, wherein the NMD exon mRNA is encoded by a genetic sequence with at least about 80%, 85%, 90%, 95%, 97%, or 100% sequence identity to SEQ ID NO: 1, 3-6, 11, and 13-16.

Embodiment 162. The composition of embodiment 159, wherein the targeted portion of the NMD exon mRNA comprises a sequence with at least 80%, 85%, 90%, 95%, 97%, or 100% sequence identity to a region comprising at least 8 contiguous nucleic acids of SEQ ID NO: 2, 7-10, 12, and 17-20.

Embodiment 163. The composition of any one of embodiments 159 to 162, wherein the targeted portion of the NMD exon mRNA (i) is within non-sense mediated RNA decay-inducing exon 20×, (ii) is upstream or downstream of non-sense mediated RNA decay-inducing exon 20×, or (iii) comprises an exon-intron junction of non-sense mediated RNA decay-inducing exon 20×.

Embodiment 164. The composition of any one of embodiments 159 to 163, wherein the therapeutic agent is an antisense oligomer (ASO) and wherein the ASO comprises a sequence that is at least about 80%, 85%, 90%, 95%, 97%, or 100% identity to any one of SEQ ID NOs: 21-114.

Embodiment 165. The composition of any one of embodiments 149 to 164, wherein the disease or condition is induced by a loss-of-function mutation in Nav1.1.

Embodiment 166. The composition of any one of embodiments 149 to 165, wherein the disease or condition is associated with haploinsufficiency of the SCN1A gene, and wherein the subject has a first allele encoding a functional SCN1A, and a second allele from which SCN1A is not produced or produced at a reduced level, or a second allele encoding a nonfunctional SCN1A or a partially functional SCN1A.

Embodiment 167. The composition of any one of embodiments 149 to 166, wherein the disease or condition is encephalopathy, optionally induced by a loss-of-function mutation in Nav1.1.

Embodiment 168. The composition of embodiment 167, wherein the encephalopathy is epileptic encephalopathy.

Embodiment 169. The composition of embodiment 165 or 166, wherein the disease or condition is Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or malignant migrating partial seizures of infancy.

Embodiment 170. The composition of embodiment 168, wherein GEFS+ is epilepsy, generalized, with febrile seizures plus, type 2.

Embodiment 171. The composition of embodiment 168, wherein the Febrile seizure is Febrile seizures, familial, 3A.

Embodiment 172. The composition of embodiment 168, wherein SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

Embodiment 173. The composition of any one of embodiments 165 to 172, wherein the therapeutic agent promotes exclusion of the NMD exon from the processed mRNA encoding SCN1A protein and increases the expression of SCN1A in the cell.

Embodiment 174. The composition of any one of embodiments 165 to 173, wherein the therapeutic agent is an antisense oligomer (ASO) and wherein the ASO comprises a sequence that is at least about 80%, 85%, 90%, 95%, 97%, or 100% complimentary to any one of SEQ ID NOs: 22-24, 26, 27, 29-35, 37-62, or 64-67.

Embodiment 175. The composition of any one of embodiments 149 to 164, wherein the disease or condition is induced by a gain-of-function mutation in Nav1.1.

Embodiment 176. The composition of any one of embodiments 149 to 164 or 175, wherein the subject has an allele from which SCN1A is produced at an increased level, or an allele encoding a mutant SCN1A that induces increased activity of Nav1.1 in the cell.

Embodiment 177. The composition of any one of embodiments 149 to 164, 175, or 176, wherein the disease or condition is migraine.

Embodiment 178. The composition of embodiment 177, wherein the migraine is migraine, familial hemiplegic, 3.

Embodiment 179. The composition of any one of embodiments 149 to 164, 175, or 176, wherein the disease or condition is a Nav1.1 genetic epilepsy.

Embodiment 180. The composition of any one of embodiments 149 to 164, or 175 to 179, wherein the therapeutic agent inhibits exclusion of the NMD exon from the processed mRNA encoding SCN1A protein and decreases the expression of SCN1A in the cell.

Embodiment 181. The composition of any one of embodiments 149 to 164, or 175 to 180, wherein the therapeutic agent is an antisense oligomer (ASO) and wherein the ASO comprises a sequence that is at least about 80%, 85%, 90%, 95%, 97%, or 100% complimentary to any one of SEQ ID NOs: 21, 25, 28, 36, or 63.

Embodiment 182. The composition of any one of embodiments 149 to 181, wherein the processed mRNA encoding the target protein or functional RNA is a full-length mature mRNA, or a wild-type mature mRNA.

Embodiment 183. The composition of any one of embodiments 149 to 182, wherein the target protein produced is full-length protein, or wild-type protein.

Embodiment 184. The composition of any one of embodiments 149 to 183, wherein the therapeutic agent is an antisense oligomer (ASO) and wherein the antisense oligomer comprises a backbone modification comprising a phosphorothioate linkage or a phosphorodiamidate linkage.

Embodiment 185. The composition of any of embodiments 149 to 184 wherein the therapeutic agent is an antisense oligomer (ASO) and wherein said antisense oligomer is an antisense oligonucleotide.

Embodiment 186. The composition of any of embodiments 149 to 185, wherein the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment 187. The composition of any of embodiments 149 to 186, wherein the therapeutic agent is an antisense oligomer (ASO) and wherein the antisense oligomer comprises at least one modified sugar moiety.

Embodiment 188. The composition of embodiment 187, wherein each sugar moiety is a modified sugar moiety.

Embodiment 189. The composition of any of embodiments 149 to 188, wherein the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment 190. A composition comprising an antisense oligomer, the antisense oligomer comprising a sequence of at least 8 contiguous nucleotides that is at least 80%, 85%, 90%, 95%, 97%, or 100% complementary to intron 20 of SCN1A.

Embodiment 191. A composition comprising an antisense oligomer, the antisense oligomer comprising a sequence of at least 8 contiguous nucleotides that is at least 80%, 85%, 90%, 95%, 97%, or 100% complementary to any one of SEQ ID NOs: 7-10.

Embodiment 192. A pharmaceutical composition comprising the therapeutic agent of any of the compositions of embodiments 149 to 191, and an excipient.

Embodiment 193. A method of treating a subject in need thereof, comprising administering the pharmaceutical composition of embodiment 192 to the subject, wherein the administering is by intrathecal injection, intracerebroventricular injection, intraperitoneal injection, intramuscular injection, subcutaneous injection, intravitreal injection, or intravenous injection.

Embodiment 194. A pharmaceutical composition comprising: an antisense oligomer that hybridizes to a target sequence of a SCN1A mRNA transcript, wherein the SCN1A mRNA transcript comprises a non-sense mediated RNA decay-inducing exon, wherein the antisense oligomer induces exclusion of the non-sense mediated RNA decay-inducing exon from the SCN1A mRNA transcript; and a pharmaceutical acceptable excipient.

Embodiment 195. The pharmaceutical composition of embodiment 194, wherein the SCN1A mRNA transcript is a SCN1A NMD exon mRNA transcript.

Embodiment 196. The pharmaceutical composition of embodiment 194 or 195, wherein the targeted portion of the SCN1A NMD exon mRNA transcript (i) is within non-sense mediated RNA decay-inducing exon 20×, (ii) is upstream or downstream of non-sense mediated RNA decay-inducing exon 20×, or (iii) comprises an exon-intron junction of non-sense mediated RNA decay-inducing exon 20×.

Embodiment 197. The pharmaceutical composition of embodiment 194 or 196, wherein the SCN1A NMD exon mRNA transcript is encoded by a genetic sequence with at least about 80%, 85%, 90%, 95%, 96%, 97%, 98%, 99% or 100% sequence identity to any one of SEQ ID NOs: 1, 3-6, 11, and 13-16.

Embodiment 198. The pharmaceutical composition of embodiment 194 or 196, wherein the SCN1A NMD exon mRNA transcript comprises a sequence with at least about 80%, 85%, 90%, 95%, 96%, 97%, 98%, 99% or 100% sequence identity to any one of SEQ ID NOs: 2, 7-10, 12, and 17-20.

Embodiment 199. The pharmaceutical composition of embodiment 194, wherein the antisense oligomer comprises a backbone modification comprising a phosphorothioate linkage or a phosphorodiamidate linkage.

Embodiment 200. The pharmaceutical composition of embodiment 194, wherein the antisense oligomer is an antisense oligonucleotide.

Embodiment 201. The pharmaceutical composition of embodiment 194, wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment 202. The pharmaceutical composition of embodiment 194, wherein the antisense oligomer comprises at least one modified sugar moiety.

Embodiment 203. The pharmaceutical composition of embodiment 194, wherein the antisense oligomer comprises from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment 204. The pharmaceutical composition of embodiment 194 or 195, wherein the antisense oligomer is at least 80%, at least 85%, at least 90%, at least 95%, at least 98%, at least 99%, or is 100% complementary to a targeted portion of the SCN1A NMD exon mRNA transcript.

Embodiment 205. The pharmaceutical composition of embodiment 194 or 195 wherein the targeted portion of the SCN1A NMD exon mRNA transcript is within a sequence selected from SEQ ID NOs: 2, 7-10, 12, and 17-20.

Embodiment 206. The pharmaceutical composition of embodiment 194, wherein the antisense oligomer comprises a nucleotide sequence that is at least about 80%, 85%, 90%, 91%, 92%, 93%, 94%, 95%, 96%, 97%, 98%, or 99% sequence identity to any one of SEQ ID NOs: 21-114.

Embodiment 207. The pharmaceutical composition of embodiment 194, wherein the antisense oligomer comprises a nucleotide sequence selected from SEQ ID NOs: 21-114.

Embodiment 208. The pharmaceutical composition of any one of the embodiments 194 to 207, wherein the pharmaceutical composition is formulated for intrathecal injection, intracerebroventricular injection, intraperitoneal injection, intramuscular injection, subcutaneous injection, intravitreal injection, or intravenous injection.

Embodiment 209. A method of inducing processing of a deficient SCN1A mRNA transcript to facilitate removal of a non-sense mediated RNA decay-inducing exon to produce a fully processed SCN1A mRNA transcript that encodes a functional form of a SCN1A protein, the method comprising:
(a) contacting an antisense oligomer to a target cell of a subject;
(b) hybridizing the antisense oligomer to the deficient SCN1A mRNA transcript, wherein the deficient SCN1A mRNA transcript is capable of encoding the functional form of a SCN1A protein and comprises at least one non-sense mediated RNA decay-inducing exon;
(c) removing the at least one non-sense mediated RNA decay-inducing exon from the deficient SCN1A mRNA transcript to produce the fully processed SCN1A mRNA transcript that encodes the functional form of SCN1A protein; and
(d) translating the functional form of SCN1A protein from the fully processed SCN1A mRNA transcript.

Embodiment 210. A method of treating a subject having a condition caused by a deficient amount or activity of SCN1A protein comprising administering to the subject an antisense oligomer comprising a nucleotide sequence with at least about 80%, 85%, 90%, 91%, 92%, 93%, 94%, 95%, 96%, 97%, 98%, or 99% sequence identity to any one of SEQ ID NOs: 24-114.

Embodiment 211. A method of screening for an agent that increases gene expression of a target protein or functional RNA by a cell, wherein the cell has an mRNA that contains a non-sense mediated RNA decay-inducing exon (NMD exon mRNA), and wherein the NMD exon mRNA encodes the target protein or functional RNA, the method comprising
(a) contacting a test agent that targets the NMD exon mRNA to a first cell;
(b) contacting a control agent to a second cell;
(c) determining a first level in the first cell, wherein the first level is a level of (i) an RNA transcript encoded by the NMD exon mRNA that does not comprise the RNA decay-inducing exon, or (ii) a protein encoded by the NMD exon mRNA that does not comprise an amino acid sequence encoded by the RNA decay-inducing exon;
(d) determining a second level in the second cell, wherein the second level is a level of (i) an RNA transcript encoded by the NMD exon mRNA that does not comprise the RNA decay-inducing exon, or (ii) a protein encoded by the NMD exon mRNA that does not comprise an amino acid sequence encoded by the RNA decay-inducing exon;
wherein the first level is higher than the second level; and
(e) selecting the test agent.

Embodiment 212. A method of screening for an agent that increases gene expression of a target protein or functional RNA by a cell, wherein the cell has an mRNA that contains a non-sense mediated RNA decay-inducing exon (NMD exon mRNA), and wherein the NMD exon mRNA encodes the target protein or functional RNA, the method comprising
(a) contacting a test agent that targets the NMD exon mRNA to a first cell;
(b) contacting a control agent to a second cell;
(c) determining a first level in the first cell, wherein the first level is a level of (i) an RNA transcript encoded by the NMD exon mRNA that comprises the RNA decay-inducing exon, or
(ii) a protein encoded by the NMD exon mRNA that comprises an amino acid sequence encoded by the RNA decay-inducing exon;
(d) determining a second level in the second cell, wherein the second level is a level of (i) an RNA transcript encoded by the NMD exon mRNA that comprises the RNA decay-inducing exon, or (ii) a protein encoded by the NMD exon mRNA that comprises an amino acid sequence encoded by the RNA decay-inducing exon;
wherein the first level is lower than the second level; and
(e) selecting the test agent.

Embodiment 213. The method of embodiment 211 or 212, wherein the method comprises contacting a protein synthesis inhibitor to the first cell and the second cell; wherein the first level is a level of an RNA transcript encoded by the NMD exon mRNA that comprises the RNA decay-inducing exon; and wherein the second level is a level of an RNA transcript encoded by the NMD exon mRNA that comprises the RNA decay-inducing exon.

Embodiment 214. A method of treating Dravet Syndrome (DS), Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP (sudden unexpected death in epilepsy) in a subject in need thereof, by increasing the expression of a target protein or functional RNA by a cell of the subject, wherein the cell has an mRNA that contains a non-sense mediated RNA decay-inducing exon (NMD exon mRNA), and wherein the NMD exon mRNA encodes the target protein or functional RNA, the method comprising contacting the cell of the subject with a therapeutic agent that modulates splicing of the NMD exon mRNA encoding the target protein or functional RNA, whereby the non-sense mediated RNA decay-inducing exon is excluded from the NMD exon mRNA encoding the target protein or functional RNA, thereby increasing the level of processed mRNA encoding the target protein or functional RNA, and increasing the expression of the target protein or functional RNA in the cell of the subject.

Embodiment 215. A method of increasing expression of SCN1A protein by a cell having an mRNA that contains a non-sense mediated RNA decay-inducing exon (NMD exon mRNA) and encodes SCN1A protein, the method comprising contacting the cell an agent that modulates splicing of the NMD exon mRNA encoding SCN1A protein, whereby the non-sense mediated RNA decay-inducing exon is excluded from the NMD exon mRNA encoding SCN1A protein, thereby increasing the level of processed mRNA encoding SCN1A protein, and increasing the expression of SCN1A protein in the cell.

Embodiment 216. The method of embodiment 214 or 215, wherein the agent
(a) binds to a targeted portion of the NMD exon mRNA encoding the target protein or functional RNA;
(b) binds to one or more components of a spliceosome; or
(c) a combination of (a) and (b).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

The present invention will be more specifically illustrated by the following Examples.

However, it should be understood that the present invention is not limited by these examples in any manner.

### Example 1: Identification of NMD-Inducing Exon Inclusion Events in SCN1A Transcripts by RNAseq Using Next Generation Sequencing

Whole transcriptome shotgun sequencing was carried out using next generation sequencing to reveal a snapshot of transcripts produced by the SCN1A gene to identify NIE inclusion events. For this purpose, polyA+ RNA from nuclear and cytoplasmic fractions of HCN (human cortical neurons) was isolated and cDNA libraries constructed using Illumina's TruSeq Stranded mRNA library Prep Kit. The libraries were pair-end sequenced resulting in 100-nucleotide reads that were mapped to the human genome (February 2009, GRCh37/hg19 assembly). The sequencing results for SCN1A are shown in FIG. 2. Briefly, FIG. 2 shows the mapped reads visualized using the UCSC genome browser (operated by the UCSC Genome Informatics Group (Center for Biomolecular Science & Engineering, University of California, Santa Cruz, 1156 High Street, Santa Cruz, CA 95064) and described by, e.g., Rosenbloom, et al., 2015, “The UCSC Genome Browser database: 2015 update,” Nucleic Acids Research 43, Database Issue, doi: 10.1093/nar/gku1177) and the coverage and number of reads can be inferred by the peak signals. The height of the peaks indicates the level of expression given by the density of the reads in a particular region. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence). Inclusion of this exon leads to a frameshift and the introduction of a premature termination codon in exon 21 rendering the transcript a target of NMD.

Exemplary SCN1A gene, pre-mRNA, exon, and intron sequences are summarized in

TABLE 2
The sequence for each exon or intron is summarized in Table 3.
Table 2. List of target SCN1A gene and pre-mRNA sequences.

| Species | SEQ ID NO. | Sequence Type |
|---|---|---|
| Human | SEQ ID NO. 1 | SCN1A gene (NC_000002.12) |
| | SEQ ID NO. 2 | SCN1A pre-mRNA (encoding e.g., |
| | | SCN1A mRNA NM_006920.5) |
| | SEQ ID NO. 3 | Exon 20 gene |
| | SEQ ID NO. 4 | Intron 20 gene |
| | SEQ ID NO. 5 | Exon 21 gene |
| | SEQ ID NO. 6 | Exon 20x gene |
| | SEQ ID NO. 7 | Exon 20 pre-mRNA |
| | SEQ ID NO. 8 | Intron 20 pre-mRNA |
| | SEQ ID NO. 9 | Exon 21 pre-mRNA |
| | SEQ ID NO. 10 | Exon 20x pre-mRNA |
| Mouse | SEQ ID NO. 11 | SCN1A gene (NC_000068.7) |
| | SEQ ID NO. 12 | SCN1A pre-mRNA (encoding e.g., |
| | | SCN1A mRNA NM_001313997.1) |
| | SEQ ID NO. 13 | Exon 21 gene |
| | SEQ ID NO. 14 | Intron 21 gene |
| | SEQ ID NO. 15 | Exon 22 gene |
| | SEQ ID NO. 16 | Exon 21x gene |
| | SEQ ID NO. 17 | Exon 21 pre-mRNA |
| | SEQ ID NO. 18 | Intron 21 pre-mRNA |
| | SEQ ID NO. 19 | Exon 22 pre-mRNA |
| | SEQ ID NO. 20 | Exon 21x pre-mRNA |

TABLE 3
Sequences of target exon or intron in SCN1A pre-mRNA transcripts.

| SEQ ID | | |
|---|---|---|
| NO. | Sequence Type | Sequence |
| SEQ ID | Exon 20 pre- | GUUUCAUUGGUCAGUUUAACAGCAAAUGCCUUGGGUUACUC |
| NO. 7 | mRNA | AGAACUUGGAGCCAUCAAAUCUCUCAGGACACUAAGAGCUC |
| | | UGAGACCUCUAAGAGCCUUAUCUCGAUUUGAAGGGAUGAGG |
| SEQ ID | Intron 20 pre- | guaagaaaaaugaaagaaccugaaguauuguauauagccaaaauuaaacuaaauuaaauuuag |
| NO. 8 | mRNA | aaaaaggaaaaucuaugcaugcaaaaggaauggcaaauucuugcaaaauugcuacuuuauugu |
| | | uuuaucuguugcauauuuacuucuaggugauaugcaagagaaauaggccucucuugaaauga |
| | | uauaauaucauuuaucugcuguccuuauuuaaaugacuuuauuuccuaauccaucuugggag |
| | | uuuccuuacaaaucuauauacaaaaaaaagcugaugcauuauuaaaguacuauguguaaugau |
| | | auaaugguaaucuaaaguaaauucuauaucagguacuuauucuuugugaugauauacuguac |
| | | uuaacgaguuuuccugaaaauaaugugaaucacacaugugccuaaguaugaguguuaagaaa |
| | | aaaaugaaaggaguuguuaaaacuuuugucuguauaaugccaaaguuugcauuauuugaaua |
| | | uauucaagauuagaugguuagauauuaaguguugacugaauuuauaaaacuaguaauacuaa |
| | | cuuaaagauuacauacaaauccacaucauuuuuauaacaauaaaguaaaacacuuauaaugaac |
| | | agaaaauauaauuuugacucauuacuauagguaauuuauacauuaaccuuaacuugcaucuua |
| | | uuggucagagucacacaaaauguuauuuuauccuuuucaaagaugcaauaaucauuuuccau |
| | | caugcauaacagauuagaaauuuugccauuauugacuuauuuuccaugccuuuuuuuacggc |
| | | augaagcauuaguuuauagauauauaauauaaaaaauuaguucugcuuuuuuuuaaaaaaaa |
| | | auauuaucaaaacaaaacacugaauugugugauuccaauagaaaaacacugcucuuucaccuc |
| | | cuaagguguaguuacuuuuauggaaacuaagcuguauuguagacuuccauuugcacuuugua |
| | | gauuguuuauagccuuauguucucuucucaagucuuauuauaaaugucacuuuguaagaacg |
| | | uaggacuugucuucgauuucccuaacauauaugaaaacuuuguccuauuaucgacaacucag |
| | | aacaauauaauacaaguaguccucuuuuauuucucacagagagccucaaauuuucaccaaaau |
| | | guuaacagaaauuaucucugggguguauaagaauuaagucuguuuuccaauuaaaugucacu |
| | | uuguuuuguuucagacuggcaguuucaguucuggagaaaaaaaaugucauuuguguacauuc |
| | | uacuugaaaacauguugccugaaucaaaauaauauauuuuauauggcuugugaaaucugaac |
| | | aaugcuaaacauuugaaaauauuauaaaccuuuuacauuugaccauuugaaguuuauuaaau |
| | | ucauuggucaagugcucagauauuuccauacauuacacuucauuucuauaaaaagcugaucu |
| | | uaucgguauacuuuuaauuuucucagaaauaaccauaucuauaauuauuaaucaauaaugccu |
| | | uuuauauuaaaagagguuaguuuuugaaacuuggaguuuuagacauaaaauccuuauaaaug |
| | | cugauagugauauaacuaauaguuuaaauggucagauuuaugaauauggcucuauuccucau |
| | | aaugacaacauacacacagcacuaaaaugacuaaucucuucaauacguguuuggcauuguaga |
| | | gucaaaauaacguuauaauugauucuauuuuuuauacuucuaguguuuggauauuuuauuu |
| | | uguaaaaauauaaucaugaaugauggugagguuggauauaagaaugaugauuaugauuggga |
| | | agugagauuugaacaugcucagaaacucucauuuaauucuuugcccuagcagcauaaaaucac |
| | | aauagcugcgucaaagcguaacucaggcacucauuuuauuuuuguuguucuguuauuuuuuc |
| | | aaagcaugugcuuuuaugcaacauuacugaauaaagcauguuguacagugcuugauaagaag |
| | | uuagaaaguaacaaauaaauuaucaucacguugcacuuuguguuuugcauguuuuaugcaca |
| | | uuucuggcugacagcuuuuaaacauuuauuguauuucaaauuuccaguccaaauuuuucaac |
| | | uuguaaaauuaaacugagugaauugaugucgugaauaucuaggguaaaauaaaauuuguguu |
| | | uaaauuuguauuuuuaauuuccuaaccuaggaaaucuuaaauaccuucuuuuucaaaagaac |
| | | ucaagucuuaauggauagggaaacagacggagagcaucaugaacaaaaaguaacaccaaaugu |
| | | ucugucauaucagauuucuaacuaauaacaaacuauauauuucuauuuuguauaggauaauc |
| | | uugcuccaacuuggaugggguggagcgcugguuccuccccugagcccuuuauuauggguacu |
| | | guauuaccccuuuugcuaccuuuaauccuugcacugugacuuauguguaguggggugaggg |
| | | agggauugggaaggguacuauuauugcaccacaguagggaaaauacauuauuuacauccuaa |
| | | uccccucuuuucaauugucuuaaauuucauuugaaaaaaaaaaaaaccuuuaugaauuuaccc |
| | | ucuguggauuuuaaccccaaugguugauaucuuuauuaaguuucauugaauaugauuuagu |
| | | uauguguauauggaguuauccaucuuuggggagauuacuggauuggugaggggggggacc |
| | | cugguguagaaugauuaugugaaaaaacaauuuaacuuguuaagcucaugauacuguuugag |
| | | gcauacagccccugcuguuuaguacauuggucuggguccugaaaauuaccaguuagauacca |
| | | ucaguugauuauugauauguaugagcagauacuagggugcaauauuucagguuucauaagac |
| | | uggauuuugugaccacucucauuuuuuauuguguaaguucauaugggguuauuuucaa |
| | | aauguuaacaaggcaaaaauauauuaagaaauaguugaauaagcacaugugaauuguguugu |
| | | aaacaaaaaguuagaauaaaaaaauccacuuauuugaauuaugcagaauagaauacauaccuag |
| | | aaauaaaacaaaaacgucuuaucaugaguauuaagauaaauuuaaggcauaaacucacuucu |
| | | uagaauaaguaacucccaacuaacuuucuaggauuuuaaaacauaacacagugaaaacauacau |
| | | aaacauaacucuacauuuuauuuauucuuaaaguuuaaguguauuauacaagaagaagaguu |
| | | uauauucgagagacagaaaaagucagaauuuuuguuuggaucaccaauauaucauagcuuaca |
| | | aaaaaacugucuuaauuaaaacccacaacauaauuuuuuuagauuuuuaagaaagauucuauu |
| | | auucuucuuuauacuuaaaaauggaugauuccuacuuugcccacuuuuauuuuuauucacau |
| | | agauuuucuuuauuucuauuagagaagcacuagaauucaugaauaguguugauuugaaguuc |
| | | aaaguaauuaauucagauaaaaagacauuucugcauguaugaaaauuucuaaugugaauuug |
| | | cauauuuaauuaucaauccuucauuuaguguagacuuauuuuuaaaaaugcagguaaugaac |
| | | cagaaauagaauugguugugcuagaguagagaaacuuuauuugaugauuguuuugaaaaaaa |
| | | agcuucugagaagaaacaaccucuaguacaguauuaauucauuaagauagcuccuuucucaga |
| | | cauuuccuuucauguagccugaaaguucaauuugaaauuuguucuuuccaauuuauucagac |
| | | uaauucugccuacuuucuuccccccauaagaaccaauuacugcagcuuuauugagacugaaaa |
| | | aaguaauacaccuccuucuuugcugaaccaaggaauggcuuggaacucuugggaaaagacaa |
| | | ucuuuucuaugaucuuucauugucuaauuuaauacaucauaaauaugacuauagcuuuguau |
| | | aauaaacuccccaauacugugccagauguuuucuaagauaaaguuauuuuguucacaaaaa |
| | | aaauaaaacuuuucucugggccaaauguaugccaacuuugcaaaucauauccugaagugcacu |
| | | gcugcagaguacaugcuugcgucauaaauuccauagaguucgcuuuaacucuaaaucaauccc |
| | | caguuucaaaguaaaccucucaaacauauuaccuaagcacaaacuucucccugugcucaguuc |
| | | cuuaauuauucucaucccauauucagaaauaacauuuaaaaauuaugcuuugaucaauaaaua |
| | | cuaaucaaacuuugcuucauuaacccauucauuuuugucaaccauuauuuuauuccuauau |
| | | ucaaagcucucugguauguucuuauauucaagacacucucaaggcccuggaagauucacgaacau |
| | | auguuugcaucuuaaauuuuuagaaaaucuuacaaucugucaggauuacacugaacucuagu |
| | | acagaguaauauggguaccagauaagugggagcaacucuuccacguagacuggaaacagcacu |
| | | aaaugcuauuuauaggcuacuuucugaacuuaacuuguuuuaaccucauuuuucucauaugc |
| | | caaaugagaacgcaauacugaauuaucuguacaguucuguucaguacuagaauucugauucu |
| | | ugaauucaaaggggaaaacauuccucuuuauuuuggaggcuaaacugggggacaaaguuagg |
| | | cuccaugaaagaagugcuauuugaacuaaagccuuuaagaggggagaguauuucagaagagg |
| | | agcuauuagacaaggaauuucaauguaaauggcaucucaaucaccuggcaauuauauuagcac |
| | | acgguuauuauauuaauugaaguggcaugaaguauagaugaccagggaaguuaaaacuggaa |
| | | auauagauuguggagugaugugaauaccaagguaagaaaaauauuuguuaguuaccagagag |
| | | ccaauaaauaacuuucaagugggacuuggggaagauuaauucaucuuacauagauuaaaugaa |
| | | ggagaagguuaggagacagaugacagugcaaguaugaaauaacagagggcaguucuaggugg |
| | | ugacugugagaauggaaaagagguggcaaagcugagaaacguuucaaagaaaaaaugugagac |
| | | agguaaugugaaaagaaaaucgagaaauagguauagauaaucaguguucugcucauacucuaa |
| | | auuggguguugaaggcaaaauacguauuuuaauuaguacucuguguauacacacuagaaaca |
| | | gcauuguaaucuggauaguggacaaaauauucagaaaagaagggaaauaguaacuugauuuc |
| | | aauuuccaaaucucaaucugaaagaaaucuaauucuauucauccauuuaaaauaaauuauau |
| | | aacgagaauuuaugaaguccauuguauuaaugcagacagucagaugagauaaggcaaagugu |
| | | cacgugucagcuugguaguugcaucggccacaucauuugguucugccuggauaacucaacca |
| | | aauuaauuuuucauacucauccccuccaccuuugucauuacugguauucuuauuuucuuugg |
| | | cccacuuaucacacuguuuuauguuccccagaaggccuagaguucuuuacaggcuuuaaacag |
| | | ggaucagaaguauaagaaauuggcucauguauuuuuuuuucagacaggcaguuaaaaaaaau |
| | | uguucuaaaaauacacuggcaucaaauggcaaauagaagauguuuugacgacuacuuccauug |
| | | gaucagacugacaagaauaauacaagcacauagguggaauuaaacuuagcuauuaauguccaa |
| | | guuugaggcagcugccccuuauaagcauuuuagggucuguuuuuagcuucccucuuagccac |
| | | uccugugcagcuccagugggagguauggaggaaaaagcaaggaagccaucccuauguuguuu |
| | | ccaaacaugaacacucaagauuuuuaacuagugguccagaaguaaagagggggaaaacauccu |
| | | ucuauagaaaaaaaaaaaaguagauaauaauugaacacagaacuucaugugaucacaucagau |
| | | uugagaacuauguauggcaucccucuuuuucuuauuuuccuaagaaaugauuucuauuaugu |
| | | uucauuugaaauaaguuuuuugaauuaaacucaguaaaugaaacaacugacaugacuggagc |
| | | uugaaauaaacgaugugaugaucuaaugaaauacauaaugcaaauugucuugcuucuuaugc |
| | | aaaaauuauuagucauagcaaugcaugaauaauuaaagacaauuauauuagguauuuaauaau |
| | | auuuuuuauauuuaucaucugaauuuuuaaguuauuuuaaaaauauauuggucaaaucaacu |
| | | cagguccaaauguuuuaguuuuguucuuuaauauauugccuuuuuaaaaugaguuaaacuuc |
| | | uguauaggcuuuuuaacuuuucuuuauucugauaacacaauucugacuucaucuggcagcaa |
| | | guuccucugauuuuccuuuuccuuuaaccuuuuaaugcuucucccucccuuuuuuuuaaaaa |
| | | cauuuuuguuucauuucuugguuauauugccuauaguuguuuuccuaaguguauugcuuaa |
| | | gaaaaaaaaaugaauuuuaagauuuuuuugaaccuugcuuuuacauauccuagaauaaauagc |
| | | auugauagaaaaaaagaauggaaagaccagagauuacuaggggaauuuuuuuucuuuauuaa |
| | | cagauaagaauucugacuuuucuuuuuuuccauuuguguauuag |
| SEQ ID | Exon 21 pre- | GUGGUUGUGAAUGCCCUUUUAGGAGCAAUUCCAUCCAUCAU |
| NO. 9 | mRNA | GAAUGUGCUUCUGGUUUGUCUUAUAUUCUGGCUAAUUUUCA |
| | | GCAUCAUGGGCGUAAAUUUGUUUGCUGGCAAAUUCUACCAC |
| | | UGUAUUAACACCACAACUGGUGACAGGUUUGACAUCGAAGA |
| | | CGUGAAUAAUCAUACUGAUUGCCUAAAACUAAUAGAAAGAA |
| | | AUGAGACUGCUCGAUGGAAAAAUGUGAAAGUAAACUUUGAU |
| | | AAUGUAGGAUUUGGGUAUCUCUCUUUGCUUCAAGUU |
| SEQ ID | Exon 20x pre- | gauaaucuugcuccacuuggaugggguggagcgcugguuccucccugagcccuuuauuau |
| NO. 10 | mRNA | gg |
| SEQ ID | Exon 21 pre- | GUUUCAUUGGUCAGUUUAACAGCAAAUGCCUUGGGUUACUC |
| NO. 17 | mRNA | UGAACUCGGGGCCAUCAAAUCCCUAAGGACACUAAGAGCUCU |
| | | GAGACCCCUAAGAGCCUUAUCACGAUUUGAAGGGAUGAGG |
| SEQ ID | Intron 21 pre- | guaagaaaaaggaaaacucugcagcguuguauauugucaaagcuaggcugaguucaacuuaac |
| NO. 18 | mRNA | uaacgaaaaacacgugcaugcaaaaggaauggcaacccuuugcaaacuugcuacuuuacccuu |
| | | uucucuguugcauauuuacuucuuggugauaugcaagagaaaaucggccucuuugaaaauga |
| | | uuuaauaucauuuaucugcuuugcuaauuaaaaugaccuuaguucauaaucgaucuugggag |
| | | uuuccuuauaauuccuaauacaaagggggaggggcagauacucucuuaaagaacuaaguuga |
| | | gucauguaauaauuaccuagagauaauuuuguuucauaucguucuccucuaugacagcccau |
| | | caguacuuaagggauccuauggaaaguaaugugaaucacaaauguguaugaauacaaaggaaa |
| | | aaaugaagaauuguuaaauguuuugucuuuacaaugccaaaauucucauuauuugaauauau |
| | | ccaagggcagauauuaaccauugacuggaguauaauaauacugccucaacuguaacuaaauua |
| | | augacauugaauaaguaagacacuaauuuaauuacuauaaauacauacacaucuuaugacaau |
| | | acagcugauaaggaaaaagaacauguauuuuuauucauugccauacauggcucgucaaccuua |
| | | acuuaaaccucgguucucaguuacacagaguuuuauguugcucuuuugagcaaagcauuauu |
| | | ccccucuccauaauucaacacguaucagauuuuugcauuuauuggcucauuguuaugaugau |
| | | uauuucaaagcauuauacaaucauuuauagaagaugugccgugugaaaauuauuuuuuuauu |
| | | aagauccaaaauuuacgcucuuuaaaccaaucagaugaaauguauaaggcaaagagugcucau |
| | | uguccugacacuuacaaaccaaggcuccaacaaacaggcuccucucugcaccacauagagggcu |
| | | uucagccuguguccccccauaaaaaccuauuauaaauuuuauuauacuauacuguaagaaacc |
| | | uguccaauuuuuaauuucucuagcacauaugaaaacuucucuucaguagauccaaguaagcac |
| | | aaaggagcuuugauucucacacaagaaaucacauuuguauuaaaauguaucauaaauuuucu |
| | | cccaauuaugcaaaacuuaaugcuuuuccaauuaaaagagcacuuucguuucagaauagcaa |
| | | uuucaguugucaaggaaaacauuguuuuuauacauuuuauauaaaaauacaugagcuaaauu |
| | | uaaauucacauuuuucaacuuuuuaugguuuuuuuauguuucuuuuucuuucgcauuuuuu |
| | | aacaaccagccauuaccucuccucccugucccccuccuauaguuucucaucucauuccuccu |
| | | cccccuugccucugagaggaugcucccucccucacuaggccucccucuuccccagggcuucaa |
| | | guuccucaaggauuauacacaucuucucccacugagaccaggccaggcaguccucugcuucug |
| | | cccagccuguguauguuccugcuugguagcucagucucuggaagcucccuggggucugggu |
| | | uaguugggacugcuggucuuccuauggaguuacccucccuucaacuucuucaauccuuccc |
| | | cuaauucaaccacagguauccagacuucaguccaaugguugaguguaaauauuggcaucugu |
| | | cucugucagcuguuguuaggggcucagaggacagccaugcuaggcuccugccuacaagcagc |
| | | acaccauaacaucaguaauagugucaggccuuuaaugaauaaugcuacguauauaagguugu |
| | | uagauuauacuucaacuuugaucuuuuagaauauuauuaaauccagucguuuauuuuuuaua |
| | | uguaauauugacuuuccauaacaaaugagucuauuuuccuuuuguguagaaauaacuuuauc |
| | | aauuauuguuaauaaugcuuuugucaauuauuguugauaugcuucucuuuuuuaaaacugg |
| | | agucaucaaaacaaaaauucuggguagauauuacgaagcugacuccuuggucagcuuggcaca |
| | | uagugagaccacaaauaucucaaggucacggcaauuccucaccaccaguuuggcauugugaag |
| | | ucaaaaccaaccuucuguugauucugguuuuuguauuucuaguaugagacauuuucuacuuu |
| | | guaagaguauauaacuguggauggcggcgagguugggcaugaugaugauuguaagcgggaa |
| | | gugagcuagaguaucuucagaaacucucacuuuauccuccuuggcagcauagaaccgcaaucg |
| | | cuguguccgagugucaaccaggcagucauuuuguuuuggguuuuuugucacucuuucaaag |
| | | caugugcuucuacgcaacacuaccaaacacagcaugcugcauagugcuugagaaggaguuaga |
| | | aaguaacaaacgaguuaucaucacguugcccuuuguguuuugcaugucuuaugcacacuuuu |
| | | ggcugacagcuuuugaacauuuauuguauuucaaauuuccaguccaaauuuuuuuuucaacu |
| | | ugugaaauugaacggaaugaaccgaugucgugaauaccuagggucaaauaaaacuuguauuu |
| | | aaauucguaguuuuaauuucccaagcugggaaaaucguaaaaaccuuuuccaaagaacucaag |
| | | ucuuaguugcuagggaaacaggcagugagcaucauauacaaaaaguaacaccaaauguucugu |
| | | cauaucagcuuucuaacuaauaauaaacuauauauuucuauuuuauauaggauaaucuugcu |
| | | ccaacuuggaugggguggagcggugguuccuccccucagcccuuuauuauggguacuguauu |
| | | accccuuuugcuaccuuuaauccuugcacugugacuuauguguagugggauugagggaggga |
| | | gugggaaggguacaauugcaccacaguagggacaauacaggauuuauuuccaaauccacuacu |
| | | uuuaaugagcuuaacuucuuuugggaaaaaaaaaguuaucucugacuuaccaucuguggau |
| | | uauaaccccagaaguacauaucuuuauuacguuucacugaauaugauuuagcuauuuauacu |
| | | ucauuguccauuuauggggaaauuacucaauuggugagggugggggacccugguguaggau |
| | | gcugaugaaaacguuuucauuugucaagcucaugguagugacagagcauauaguccuuauuu |
| | | uuucaacacacugcucuggucccucaaugggccagucacauuccaucaguugaucguugaug |
| | | ugugcgagcaguggcuaaagguacaacaggccagguaucucaggguugccaaugguuaugau |
| | | cauucucaucuuuauugcauaaaaauguguuauuugcagaaaguagcaaggcaagaucccug |
| | | ugaaacaagggaanacaaaaaaaaaaaaagaugugcuuuaaguuauaaaaccaaaacaugugaa |
| | | aagucaacuucauugaaguauaaagaauaggauaugcaugaaaaacaaaaaaaucaugagcac |
| | | uaagaaauugguguauaagccaacuccuuguaagcuaccccaauuaacuucccagaaucuuag |
| | | aaggcaucacagugcaccccaaaauaaaaagccaaacugacacuucugcuuccucuuaaaaugu |
| | | aggagucuuggauaagaaagauaauuuuuauuguuuggaagaaaaaaaaauuguuuggauaa |
| | | uugaggcauuuaucuaucaaaaauauuuaucuuaauaaaauuucacaacacugauuuaguug |
| | | uuggcuuuucuaaaaauuuuuuauauuucauauuaagaacucaugauuuuuacuuuccauuu |
| | | uuuaaauucuuauucacauaugguuuuucucuauuucuuagaaaagcuauagaacccauggu |
| | | uuccgugacuuaaaaaacuaaucuaaaugucuucacuuagaugauacuuucaaaugcacuga |
| | | aauuucuaauaucaacaagaauauuugccugguccuaauuuuucacugauuuaauaaaaagua |
| | | ugaacccuaaagaagaaauagacuugaagaacugguugugugacaauacagaaauucugcugg |
| | | gagauguccuuuuaaaacauuguuagaagagacaaccucuacaauccacccauuaagcauacu |
| | | ucucucuuagacaucuccuuuuauguaccuuauaaccucaauguguucuuuccaauugacuu |
| | | agaccaacacuucccagcgcaucccacaugggagccaauuacugacucucccuagagacugcaa |
| | | agaauuaauauuguagaaccaagggaugguugggcucuugggagaggcaauccguuugugau |
| | | cuuuugcucuggauguuaaugaaaucgcaacuuuaaguggauuuucaguggcaaauccucug |
| | | auccuaugccaaauguucucuaagacaaacauccuuguaaaauaaaugucucacugggccaaa |
| | | ucuauggcaaauuugcacguuuuccugaacugcauuccuauauaguauuugccauccugaau |
| | | ucacuaaugggcauuacuuuuaauucaaaaccagucccuucuucaaaggaaaucucucccauu |
| | | uauuacauuaugcaaacugcuuucuuaugcagugguuaaauccuuagccaggcaaguaugag |
| | | gacuggaauuuggauauccagaacccucagaaaugucggaugggcauaguagcuuacaugua |
| | | auuccagagcuagaaagaugaaacuagcccuguccucaagcucugaauucaguuggugcaaua |
| | | aauaagaaagaauccccccccccgaccccuacaucucuuuccucuacaucuauacauguauuuc |
| | | ccauacagcucuaugcuuccacauauaugcucacauaugugcaugcacacuugcacacauaug |
| | | uacacuugacacauugaagcaucauaaucuaacaauuggaauauaagaaaauauuuaacuuuc |
| | | acacagagcagucagagaaaaccauaagagguugaaacucuugaaaaaacuugcaggauaaaca |
| | | gaaaagaguaugagaugccaauucuggucuacuuucugaaccuaacuuguuuuaccuucauu |
| | | agucuaauuuuccaaaugaaccccaagcaccaaauuguccuuauugcucuuuccaguacuaaa |
| | | uuauaauuccucaauucgaaggcaaacacucucaucuuuguuaaggauguguagaguuagac |
| | | ucaauaaacgacauguauauuugagcuaaagccuggaggagggagauuauuucaagggcaag |
| | | uacuuggcagggaguuucaaagaaagaaggcucucaauucucagacuaacaccuuagcgugga |
| | | gugacugucacagaggagggugaaguguaugucaccaguuagggaagcugaaaggggaaaug |
| | | uaucaggcuugugagaauccuucagcagecaagucuagcaccugagcacaauccccagaacug |
| | | accccacaugguggaaaggagaggaauaauuccugcaaauuuuccugugaccuccacccaagu |
| | | gcuauagaaaaugcaugcaugcccacaugcacacacaaauaaacauaguugcaaacuguuuug |
| | | aggaaaauaaccucacaaacugucgagugauguaaaugccaaagaaagagaaauguuuucuaa |
| | | uggcuagagaaccauuaaggaauuuuucaaaaugggacaugggauagauaaauuuaguaucc |
| | | auacugaaagaaggcaagcaaauaaaaucugauaagaauguaauucuuaguaaccgaggacag |
| | | agcgaagauagagaacagggccaauggccaagguggaaagguuugaaggaagcagcaugaaac |
| | | cuacagacuuguaccaaaauguucagugucaugaguguuaaaagugaaaagcuugcauguua |
| | | guauggauuucauauccucggcagacagagcaccucacugugagugggagaugaaguauuca |
| | | gaaaugagaaacaacuacucaauuucaguguucauaucucaaauccaauaaacaacuuuaggg |
| | | guacaauuuuuuaaaaaauuacauuaaaauguuuuuaaaucucuucuuaauaauuuaaaaau |
| | | uaaauugaaaauaacuuuaaaaaguaauauauacaggaaagccugugugcuaauuuuuuagg |
| | | gaggccauaaagggagauaguugcucauuaauuucuacacaucagecuaucuuuggcuucug |
| | | ccuugauagcgcacucugaauuaucuucuucauguucaucccucaucuuuauuguuacuggu |
| | | uucauuucccuuggccacauagcccacuauuuuguauuccccaaugguauuguuccuuaca |
| | | aaguucagccagggcucagaaguacaaggaauuggcucuuauacuucugucagacaggcaaaa |
| | | acuucuaaaauuauacuauaauaaaaaucaaagagaugauauucauaauuaaacuaacaaaagu |
| | | ggcaggcccccccucccccaacaugaguagaauuaaucugacguccauguucaagucugaaac |
| | | acacuugccaauuaagagcacauuagggccagccuuuaucucccucuuaguuacuaaugugca |
| | | guucaauggugagcuauagagaaggaagccaagacuaccauaugucaaauauaaaaaaaaaaa |
| | | aucccauuuuaaaucuguagucccgaauuaaggacaagagagagggaaauaucuuugacauua |
| | | gaaaauggagaaaauauuuuagcacaggacuuuacucagucacaucagaguugauaaguacgu |
| | | augacaucccucuuuuuccuguuuuccugagaaaaugaucucuaguguuucauuuaagau |
| | | aaguuuauugaauuaaacucaguaaaugaaacaacugacaugacuggagcuugaaauaaacga |
| | | ugugaugaucuacugaaauacaugaugcuaaauugucuugcuucuuaugcaaaaacuacuau |
| | | uaguuauagcaaugcauggauaauuaaggccaaaaauauauuagauguuaaaaauaguuuua |
| | | uauuuauacaucugaauuuuaauuuauauuuaaaguauauugguccaaucaauucaugccca |
| | | aauguuuuaguucuauucuuugagauacuguuuuguuuugggauuuuuuuuuaugagcuaa |
| | | ucucuugccuaggaguuccuacuucucucuccuccuuuuauuuuuucuaauaaacuacacau |
| | | gugucuucauccaggagcuaacuucuccauuuugcuuuuccuuuagcaccuuuuuuauauu |
| | | agauuucuuucuuuucuccaucucuuugcauauugccuauauuucuuuuccuaagcauaaua |
| | | uuuaaaaaagacugaguuuuauguuaagauuauuucugcuuugcucuuacacagauaggaua |
| | | aguagucuugauagaaaauaaaucaaugauuccuagggggaugucuuuuugcuuuuaaucaa |
| | | uaaggauucugacuucucucuuucucuccauuuguguauuag |
| SEQ ID | Exon 22 pre- | GUGGUUGUGAAUGCCCUGUUAGGAGCAAUUCCAUCCAUCAU |
| NO. 19 | mRNA | GAAUGUGCUUCUGGUUUGCCUUAUAUUCUGGCUAAUUUUCA |
| | | GCAUCAUGGGCGUAAAUUUGUUUGCUGGCAAAUUCUACCAC |
| | | UGUGUUAACACCACAACUGGUGACAUAUUUGAGAUCAGCGA |
| | | AGUCAAUAAUCAUUCUGAUUGCCUAAAACUAAUAGAAAGAA |
| | | AUGAGACCGCCCGGUGGAAAAAUGUGAAAGUAAACUUUGAU |
| | | AAUGUAGGAUUUGGGUAUCUUUCUUUGCUUCAAGUU |
| SEQ ID | Exon 21x pre- | gauaaucuugcuccaacuuggaugggguggagcggugguuccuccccucagcccuuuauuau |
| NO. 20 | mRNA | gg |

### Example 2: Confirmation of NIE Via Cycloheximide Treatment

RT-PCR analysis using cytoplasmic RNA from DMSO-treated (CHX−) or cycloheximide-treated (CHX+) mouse Neuro 2A cells (FIG. 3A) and RenCell VM (human neuroprogenitor cells) (FIG. 3B) and primers in exon 20 and exon 23 confirmed the presence of a band corresponding to the NMD-inducing exon (20×). The identity of the product was confirmed by sequencing. Densitometry analysis of the bands was performed to calculate percent exon 20× inclusion of total SCN1A transcript. Treatment of RenCell VM with cycloheximide (CHX+) to inhibit NMD led to a 2-fold increase of the product corresponding to the NMD-inducing exon 20× in the cytoplasmic fraction (cf. light grey bar, CHX−, to dark grey bar, CHX+).

### Example 3: SCN1A Exon 20× Region ASO Walk

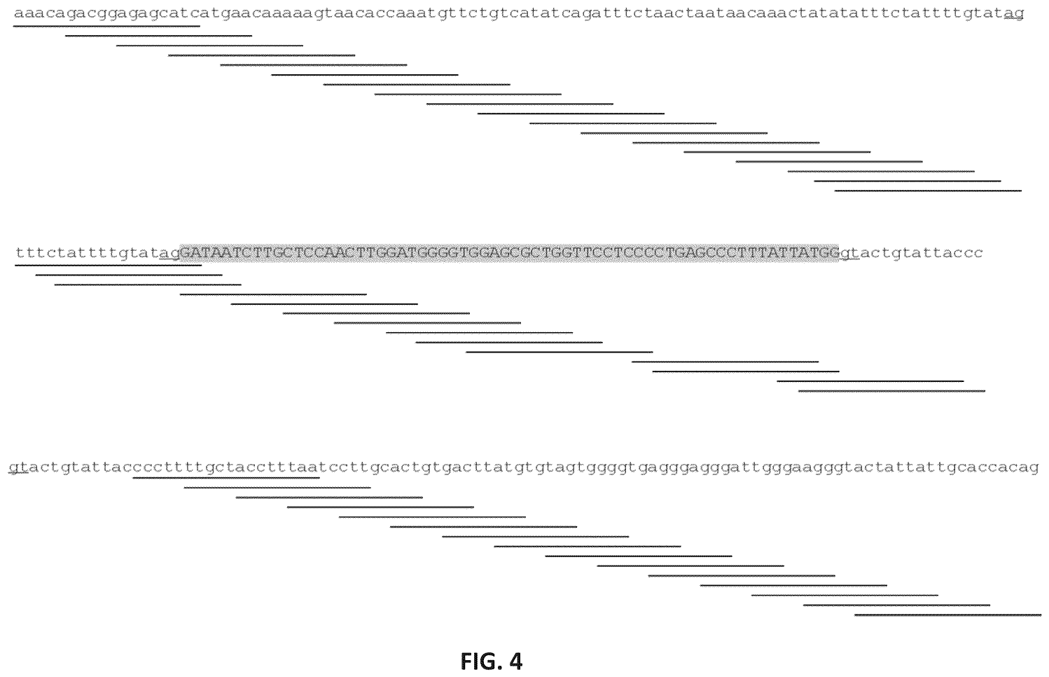

A graphic representation of the ASO walk performed for SCNA exon 20× region targeting sequences immediately upstream of the 3′ splice site, across the 3′splice site, exon 20×, across the 5′ splice site, and downstream of the 5′ splice site using 2′-MOE ASOs, PS backbone, is shown in FIG. 4. ASOs were designed to cover these regions by shifting 5 nucleotides at a time. A list of ASOs targeting SCN1A is summarized in Table 4. Sequences of ASOs are summarized in Table 5a and Table 5b and Table 6a and Table 6b.

TABLE 4
List of ASOs targeting SCN1A

| Gene | Pre-mRNA | ASOs | |
|---|---|---|---|
| SEQ ID NO. | SEQ ID NO. | SEQ ID NO. | NIE |
| SEQ ID NO. 1 | SEQ ID NO. 2 | SEQ ID NOs: | Exon 20x |
| | | 21-67, 210-256 | |
| SEQ ID NO. 11 | SEQ ID NO. 12 | SEQ ID NOs: | Exon 21x |
| | | 68-114, 257-303 | |

TABLE 5a
Sequences of ASOs targeting human SCN1A

| SEQ ID NO. | Sequence name | ASO sequence |
|---|---|---|
| 21 | SCN1A-IVS19X−81 | GATGCTCTCCGTCTGTTT |
| 22 | SCN1A-IVS19X−76 | TTCATGATGCTCTCCGTC |
| 23 | SCN1A-IVS19X−71 | TTTTGTTCATGATGCTCT |
| 24 | SCN1A-IVS19X−66 | TTACTTTTTGTTCATGAT |
| 25 | SCN1A-IVS19X−61 | TGGTGTTACTTTTTGTTC |
| 26 | SCN1A-IVS19X−56 | ACATTTGGTGTTACTTTT |
| 27 | SCN1A-IVS19X−51 | ACAGAACATTTGGTGTTA |
| 28 | SCN1A-IVS19X−46 | ATATGACAGAACATTTGG |
| 29 | SCN1A-IVS19X−41 | ATCTGATATGACAGAACA |
| 30 | SCN1A-IVS19X−36 | TAGAAATCTGATATGACA |
| 31 | SCN1A-IVS19X−31 | TTAGTTAGAAATCTGATA |
| 32 | SCN1A-IVS19X−26 | TGTTATTAGTTAGAAATC |
| 33 | SCN1A-IVS19X−21 | TAGTTTGTTATTAGTTAG |
| 34 | SCN1A-IVS19X−16 | ATATATAGTTTGTTATTA |
| 35 | SCN1A-IVS19X−11 | TAGAAATATATAGTTTGT |
| 36 | SCN1A-IVS19X−6 | CAAAATAGAAATATATAG |
| 37 | SCN1A-IVS19X−3 | ATACAAAATAGAAATATA |
| 38 | SCN1A-IVS19X−1 | CTATACAAAATAGAAATA |
| 39 | SCN1A-I19X/E20X+2 | TCCTATACAAAATAGAAA |
| 40 | SCN1A-I19X/E20X+4 | TATCCTATACAAAATAGA |
| 41 | SCN1A-I19X/E20X+6 | ATTATCCTATACAAAATA |
| 42 | SCN1A-Ex20X+1 | AGTTGGAGCAAGATTATC |
| 43 | SCN1A-Ex20X+6 | ATCCAAGTTGGAGCAAGA |
| 44 | SCN1A-Ex20X+11 | ACCCCATCCAAGTTGGAG |
| 45 | SCN1A-Ex20X+16 | GCTCCACCCCATCCAAGT |
| 46 | SCN1A-Ex20X+21 | CCAGCGCTCCACCCCATC |
| 47 | SCN1A-Ex20X−24 | GAACCAGCGCTCCACCCC |
| 48 | SCN1A-Ex20X−19 | GGGAGGAACCAGCGCTCC |
| 49 | SCN1A-Ex20X−3 | ATAATAAAGGGCTCAGGG |
| 50 | SCN1A-Ex20X−1 | CCATAATAAAGGGCTCAG |
| 51 | SCN1A-E20X/I20X−6 | GTAATACAGTACCCATAA |
| 52 | SCN1A-E20X/I20X−4 | GGGTAATACAGTACCCAT |
| 53 | SCN1A-IVS20X+13 | TTAAAGGTAGCAAAAGGG |
| 54 | SCN1A-IVS20X+18 | AAGGATTAAAGGTAGCAA |
| 55 | SCN1A-IVS20X+23 | AGTGCAAGGATTAAAGGT |
| 56 | SCN1A-IVS20X+28 | GTCACAGTGCAAGGATTA |
| 57 | SCN1A-IVS20X+33 | CATAAGTCACAGTGCAAG |
| 58 | SCN1A-IVS20X+38 | CTACACATAAGTCACAGT |
| 59 | SCN1A-IVS20X+43 | CCCCACTACACATAAGTC |
| 60 | SCN1A-IVS20X+48 | CCTCACCCCACTACACAT |
| 61 | SCN1A-IVS20X+53 | CCCTCCCTCACCCCACTA |
| 62 | SCN1A-IVS20X+58 | CCAATCCCTCCCTCACCC |
| 63 | SCN1A-IVS20X+63 | CCTTCCCAATCCCTCCCT |
| 64 | SCN1A-IVS20X+68 | AGTACCCTTCCCAATCCC |
| 65 | SCN1A-IVS20X+73 | ATAATAGTACCCTTCCCA |
| 66 | SCN1A-IVS20X+78 | GTGCAATAATAGTACCCT |
| 67 | SCN1A-IVS20X+83 | CTGTGGTGCAATAATAGT |

TABLE 5b
Sequences of ASOs targeting human SCN1A

| SEQ ID NO. | Sequence name | ASO sequence |
|---|---|---|
| 210 | SCN1A-IVS19X−81 | GAUGCUCUCCGUCUGUUU |
| 211 | SCN1A-IVS19X−76 | UUCAUGAUGCUCUCCGUC |
| 212 | SCN1A-IVS19X−71 | UUUUGUUCAUGAUGCUCU |
| 213 | SCN1A-IVS19X−66 | UUACUUUUUGUUCAUGAU |
| 214 | SCN1A-IVS19X−61 | UGGUGUUACUUUUUGUUC |
| 215 | SCN1A-IVS19X−56 | ACAUUUGGUGUUACUUUU |
| 216 | SCN1A-IVS19X−51 | ACAGAACAUUUGGUGUUA |
| 217 | SCN1A-IVS19X−46 | AUAUGACAGAACAUUUGG |
| 218 | SCN1A-IVS19X−41 | AUCUGAUAUGACAGAACA |
| 219 | SCN1A-IVS19X−36 | UAGAAAUCUGAUAUGACA |
| 220 | SCN1A-IVS19X−31 | UUAGUUAGAAAUCUGAUA |
| 221 | SCN1A-IVS19X−26 | UGUUAUUAGUUAGAAAUC |
| 222 | SCN1A-IVS19X−21 | UAGUUUGUUAUUAGUUAG |
| 223 | SCN1A-IVS19X−16 | AUAUAUAGUUUGUUAUUA |
| 224 | SCN1A-IVS19X−11 | UAGAAAUAUAUAGUUUGU |
| 225 | SCN1A-IVS19X−6 | CAAAAUAGAAAUAUAUAG |
| 226 | SCN1A-IVS19X−3 | AUACAAAAUAGAAAUAUA |
| 227 | SCN1A-IVS19X−1 | CUAUACAAAAUAGAAAUA |
| 228 | SCN1A-I19X/E20X+2 | UCCUAUACAAAAUAGAAA |
| 229 | SCN1A-I19X/E20X+4 | UAUCCUAUACAAAAUAGA |
| 230 | SCN1A-I19X/E20X+6 | AUUAUCCUAUACAAAAUA |
| 231 | SCN1A-Ex20X+1 | AGUUGGAGCAAGAUUAUC |
| 232 | SCN1A-Ex20X+6 | AUCCAAGUUGGAGCAAGA |
| 233 | SCN1A-Ex20X+11 | ACCCCAUCCAAGUUGGAG |
| 234 | SCN1A-Ex20X+16 | GCUCCACCCCAUCCAAGU |
| 235 | SCN1A-Ex20X+21 | CCAGCGCUCCACCCCAUC |
| 236 | SCN1A-Ex20X−24 | GAACCAGCGCUCCACCCC |
| 237 | SCN1A-Ex20X−19 | GGGAGGAACCAGCGCUCC |
| 238 | SCN1A-Ex20X−3 | AUAAUAAAGGGCUCAGGG |
| 239 | SCN1A-Ex20X−1 | CCAUAAUAAAGGGCUCAG |
| 240 | SCN1A-E20X/I20X−6 | GUAAUACAGUACCCAUAA |
| 241 | SCN1A-E20X/I20X−4 | GGGUAAUACAGUACCCAU |
| 242 | SCN1A-IVS20X+13 | UUAAAGGUAGCAAAAGGG |
| 243 | SCN1A-IVS20X+18 | AAGGAUUAAAGGUAGCAA |
| 244 | SCN1A-IVS20X+23 | AGUGCAAGGAUUAAAGGU |
| 245 | SCN1A-IVS20X+28 | GUCACAGUGCAAGGAUUA |
| 246 | SCN1A-IVS20X+33 | CAUAAGUCACAGUGCAAG |
| 247 | SCN1A-IVS20X+38 | CUACACAUAAGUCACAGU |
| 248 | SCN1A-IVS20X+43 | CCCCACUACACAUAAGUC |
| 249 | SCN1A-IVS20X+48 | CCUCACCCCACUACACAU |
| 250 | SCN1A-IVS20X+53 | CCCUCCCUCACCCCACUA |
| 251 | SCN1A-IVS20X+58 | CCAAUCCCUCCCUCACCC |
| 252 | SCN1A-IVS20X+63 | CCUUCCCAAUCCCUCCCU |
| 253 | SCN1A-IVS20X+68 | AGUACCCUUCCCAAUCCC |
| 254 | SCN1A-IVS20X+73 | AUAAUAGUACCCUUCCCA |
| 255 | SCN1A-IVS20X+78 | GUGCAAUAAUAGUACCCU |
| 256 | SCN1A-IVS20X+83 | CUGUGGUGCAAUAAUAGU |

TABLE 6a
Sequences of ASOs targeting mouse SCN1A

| SEQ ID NO. | Sequence name | ASO sequence |
|---|---|---|
| 68 | mScn1a-IVS20X−81 | GATGCTCACTGCCTGTTT |
| 69 | mScn1a-IVS20X−76 | TATATGATGCTCACTGCC |
| 70 | mScn1a-IVS20X−71 | TTTTGTATATGATGCTCA |
| 71 | mScn1a-IVS20X−66 | TTACTTTTTGTATATGAT |
| 72 | mScn1a-IVS20X−61 | TGGTGTTACTTTTTGTAT |
| 73 | mScn1a-IVS20X−56 | ACATTTGGTGTTACTTTT |
| 74 | mScn1a-IVS20X−51 | ACAGAACATTTGGTGTTA |
| 75 | mScn1a-IVS20X−46 | ATATGACAGAACATTTGG |
| 76 | mScn1a-IVS20X−41 | AGCTGATATGACAGAACA |
| 77 | mScn1a-IVS20X−36 | TAGAAAGCTGATATGACA |
| 78 | mScn1a-IVS20X−31 | TTAGTTAGAAAGCTGATA |
| 79 | mScn1a-IVS20X−26 | TATTATTAGTTAGAAAGC |
| 80 | mScn1a-IVS20X−21 | TAGTTTATTATTAGTTAG |
| 81 | mScn1a-IVS20X−16 | ATATATAGTTTATTATTA |
| 82 | mScn1a-IVS20X−11 | TAGAAATATATAGTTTAT |
| 83 | mScn1a-IVS20X−6 | TAAAATAGAAATATATAG |
| 84 | mScn1a-IVS20X−3 | ATATAAAATAGAAATATA |
| 85 | mScn1a-IVS20X−1 | CTATATAAAATAGAAATA |
| 86 | mScn1a-I20X/E21X+2 | TCCTATATAAAATAGAAA |
| 87 | mScn1a-I20X/E21X+4 | TATCCTATATAAAATAGA |
| 88 | mScn1a-I20X/E21X+6 | ATTATCCTATATAAAATA |
| 89 | mScn1a-Ex21X+1 | AGTTGGAGCAAGATTATC |
| 90 | mScn1a-Ex21X+6 | ATCCAAGTTGGAGCAAGA |
| 91 | mScn1a-Ex21X+11 | ACCCCATCCAAGTTGGAG |
| 92 | mScn1a-Ex21X+16 | GCTCCACCCCATCCAAGT |
| 93 | mScn1a-Ex21X+21 | CCACCGCTCCACCCCATC |
| 94 | mScn1a-Ex21X−24 | GAACCACCGCTCCACCCC |
| 95 | mScn1a-Ex21X−19 | GGGAGGAACCACCGCTCC |
| 96 | mScn1a-Ex21X−3 | ATAATAAAGGGCTGAGGG |
| 97 | mScn1a-Ex21X−1 | CCATAATAAAGGGCTGAG |
| 98 | mScn1a-E21X/I21X−6 | GTAATACAGTACCCATAA |
| 99 | mScn1a-E21X/I21X−4 | GGGTAATACAGTACCCAT |
| 100 | mScn1a-IVS21X+13 | TTAAAGGTAGCAAAAGGG |
| 101 | mScn1a-IVS21X+18 | AAGGATTAAAGGTAGCAA |
| 102 | mScn1a-IVS21X+23 | AGTGCAAGGATTAAAGGT |
| 103 | mScn1a-IVS21X+28 | GTCACAGTGCAAGGATTA |
| 104 | mScn1a-IVS21X+33 | CATAAGTCACAGTGCAAG |
| 105 | mScn1a-IVS21X+38 | CTACACATAAGTCACAGT |
| 106 | mScn1a-IVS21X+43 | TCCCACTACACATAAGTC |
| 107 | mScn1a-IVS21X+48 | CTCAATCCCACTACACAT |
| 108 | mScn1a-IVS21X+53 | CCTCCCTCAATCCCACTA |
| 109 | mScn1a-IVS21X+58 | CACTCCCTCCCTCAATCC |
| 110 | mScn1a-IVS21X+63 | CTTCCCACTCCCTCCCTC |
| 111 | mScn1a-IVS21X+68 | GTACCCTTCCCACTCCCT |
| 112 | mScn1a-IVS21X+73 | CAATTGTACCCTTCCCAC |
| 113 | mScn1a-IVS21X+78 | TGGTGCAATTGTACCCTT |
| 114 | mScn1a-IVS21X+83 | TACTGTGGTGCAATTGTA |

TABLE 6b
Sequences of ASOs targeting mouse SCN1A

| SEQ ID NO. | Sequence name | ASO sequence |
|---|---|---|
| 257 | mScn1a-IVS20X−81 | GAUGCUCACUGCCUGUUU |
| 258 | mScn1a-IVS20X−76 | UAUAUGAUGCUCACUGCC |
| 259 | mScn1a-IVS20X−71 | UUUUGUAUAUGAUGCUCA |
| 260 | mScn1a-IVS20X−66 | UUACUUUUUGUAUAUGAU |
| 261 | mScn1a-IVS20X−61 | UGGUGUUACUUUUUGUAU |
| 262 | mScn1a-IVS20X−56 | ACAUUUGGUGUUACUUUU |
| 263 | mScn1a-IVS20X−51 | ACAGAACAUUUGGUGUUA |
| 264 | mScn1a-IVS20X−46 | AUAUGACAGAACAUUUGG |
| 265 | mScn1a-IVS20X−41 | AGCUGAUAUGACAGAACA |
| 266 | mScn1a-IVS20X−36 | UAGAAAGCUGAUAUGACA |
| 267 | mScn1a-IVS20X−31 | UUAGUUAGAAAGCUGAUA |
| 268 | mScn1a-IVS20X−26 | UAUUAUUAGUUAGAAAGC |
| 269 | mScn1a-IVS20X−21 | UAGUUUAUUAUUAGUUAG |
| 270 | mScn1a-IVS20X−16 | AUAUAUAGUUUAUUAUUA |
| 271 | mScn1a-IVS20X−11 | UAGAAAUAUAUAGUUUAU |
| 272 | mScn1a-IVS20X−6 | UAAAAUAGAAAUAUAUAG |
| 273 | mScn1a-IVS20X−3 | AUAUAAAAUAGAAAUAUA |
| 274 | mScn1a-IVS20X−1 | CUAUAUAAAAUAGAAAUA |
| 275 | mScn1a-I20X/E21X+2 | UCCUAUAUAAAAUAGAAA |
| 276 | mScn1a-I20X/E21X+4 | UAUCCUAUAUAAAAUAGA |
| 277 | mScn1a-I20X/E21X+6 | AUUAUCCUAUAUAAAAUA |
| 278 | mScn1a-Ex21X+1 | AGUUGGAGCAAGAUUAUC |
| 279 | mScn1a-Ex21X+6 | AUCCAAGUUGGAGCAAGA |
| 280 | mScn1a-Ex21X+11 | ACCCCAUCCAAGUUGGAG |
| 281 | mScn1a-Ex21X+16 | GCUCCACCCCAUCCAAGU |
| 282 | mScn1a-Ex21X+21 | CCACCGCUCCACCCCAUC |
| 283 | mScn1a-Ex21X−24 | GAACCACCGCUCCACCCC |
| 284 | mScn1a-Ex21X−19 | GGGAGGAACCACCGCUCC |
| 285 | mScn1a-Ex21X−3 | AUAAUAAAGGGCUGAGGG |
| 286 | mScn1a-Ex21X−1 | CCAUAAUAAAGGGCUGAG |
| 287 | mScn1a-E21X/I21X−6 | GUAAUACAGUACCCAUAA |
| 288 | mScn1a-E21X/I21X−4 | GGGUAAUACAGUACCCAU |
| 289 | mScn1a-IVS21X+13 | UUAAAGGUAGCAAAAGGG |
| 290 | mScn1a-IVS21X+18 | AAGGAUUAAAGGUAGCAA |
| 291 | mScn1a-IVS21X+23 | AGUGCAAGGAUUAAAGGU |
| 292 | mScn1a-IVS21X+28 | GUCACAGUGCAAGGAUUA |
| 293 | mScn1a-IVS21X+33 | CAUAAGUCACAGUGCAAG |
| 294 | mScn1a-IVS21X+38 | CUACACAUAAGUCACAGU |
| 295 | mScn1a-IVS21X+43 | UCCCACUACACAUAAGUC |
| 296 | mScn1a-IVS21X+48 | CUCAAUCCCACUACACAU |
| 297 | mScn1a-IVS21X+53 | CCUCCCUCAAUCCCACUA |
| 298 | mScn1a-IVS21X+58 | CACUCCCUCCCUCAAUCC |
| 299 | mScn1a-IVS21X+63 | CUUCCCACUCCCUCCCUC |
| 300 | mScn1a-IVS21X+68 | GUACCCUUCCCACUCCCU |
| 301 | mScn1a-IVS21X+73 | CAAUUGUACCCUUCCCAC |
| 302 | mScn1a-IVS21X+78 | UGGUGCAAUUGUACCCUU |
| 303 | mScn1a-IVS21X+83 | UACUGUGGUGCAAUUGUA |

### Example 4: SCN1A Exon 20× Region ASO Walk Evaluated by RT-PCR

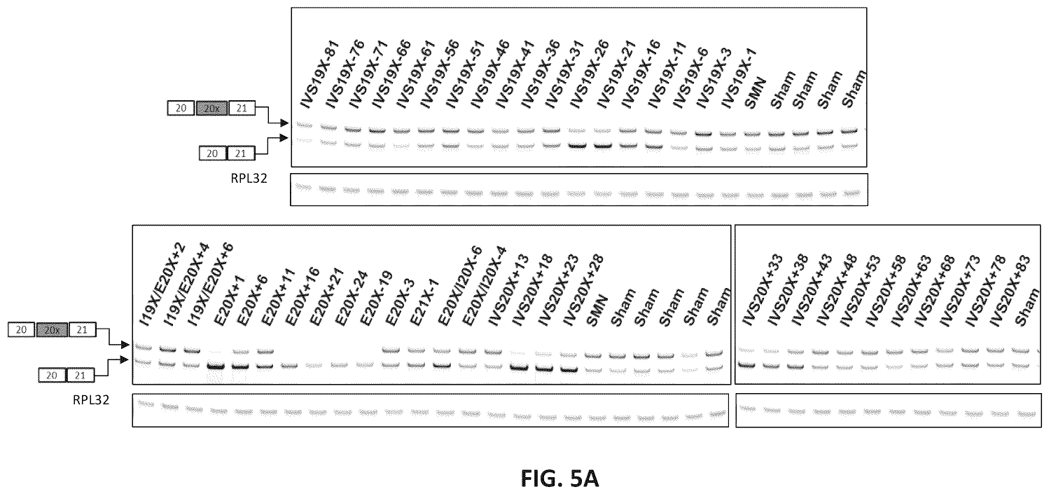

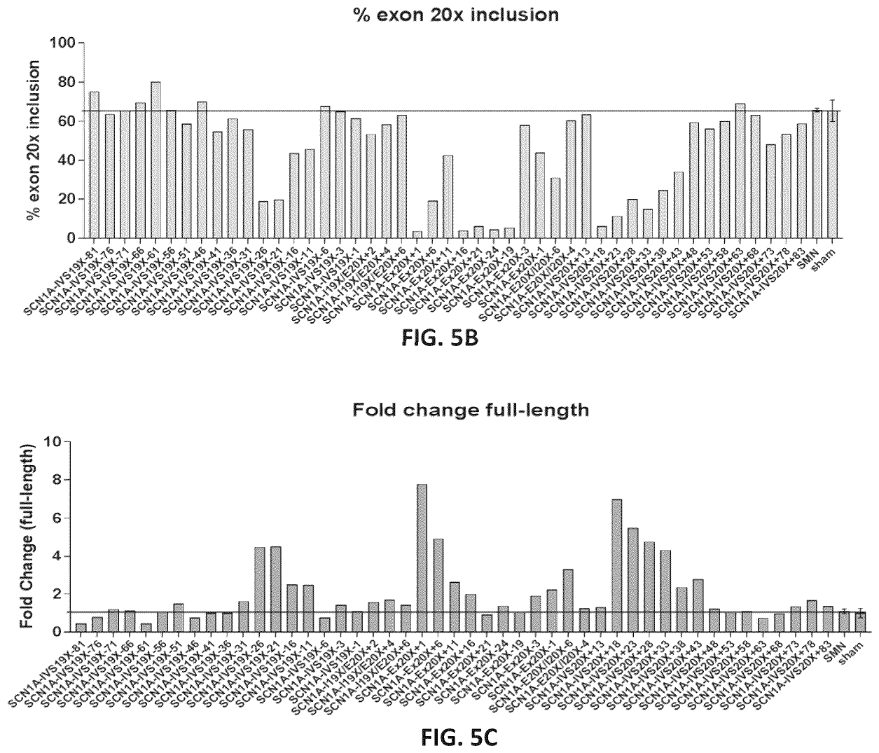

ASO walk sequences can be evaluated by for example RT-PCR. In FIG. 5A, a representative PAGE shows SYBR-safe-stained RT-PCR products of SCN1A mock-treated (Sham), SMN-control ASO treated (SMN), or treated with a 2′-MOE ASO targeting the exon 20× region as described herein in the Example 3 and in the description of FIG. 4, at 20 μM concentration in RenCell VM cells by gymnotic uptake. Two products corresponding to exon 20× inclusion (top band) and full-length (exon 20× exclusion, bottom band) were quantified and percent exon 20× inclusion is plotted in the bar graph (FIG. 5B). The black line indicates no change with respect to Sham. The full-length products were also normalized to RPL32 internal control and fold-change relative to Sham is plotted in the bar graph (FIG. 5C). The black line indicates a ratio of 1 and no change with respect to Sham.

### Example 5: SCN1A Exon 20× Region ASO Walk Evaluated by RT-qPCR

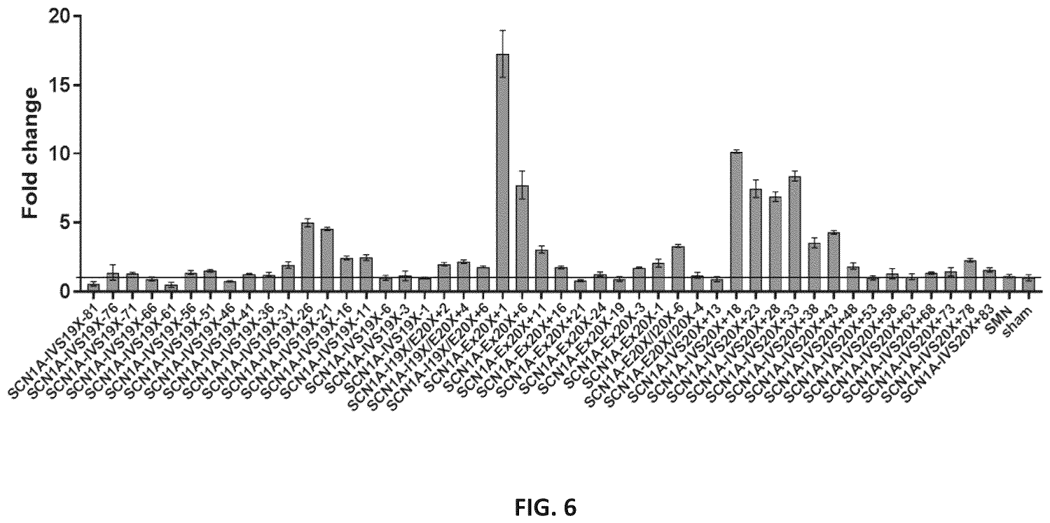

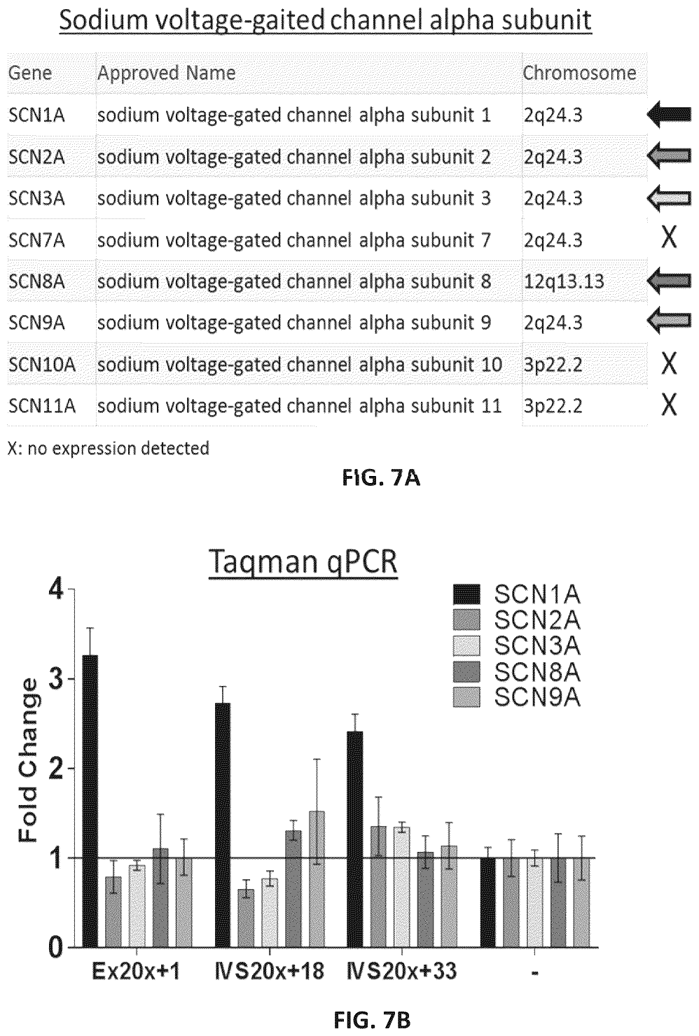

SYBR-green RT-qPCR SCN1A amplification results normalized to RPL32, obtained using the same ASO uptake experiment that were evaluated by SYBR-safe RT-PCR as shown in FIG. 6, are plotted as fold change relative to Sham confirming the SYBR-safe RT-PCR results. The black line indicates a ratio of 1 (no change with respect to sham).

### Example 6: Dose-Dependent Effect of Selected ASO in CXH-Treated Cells

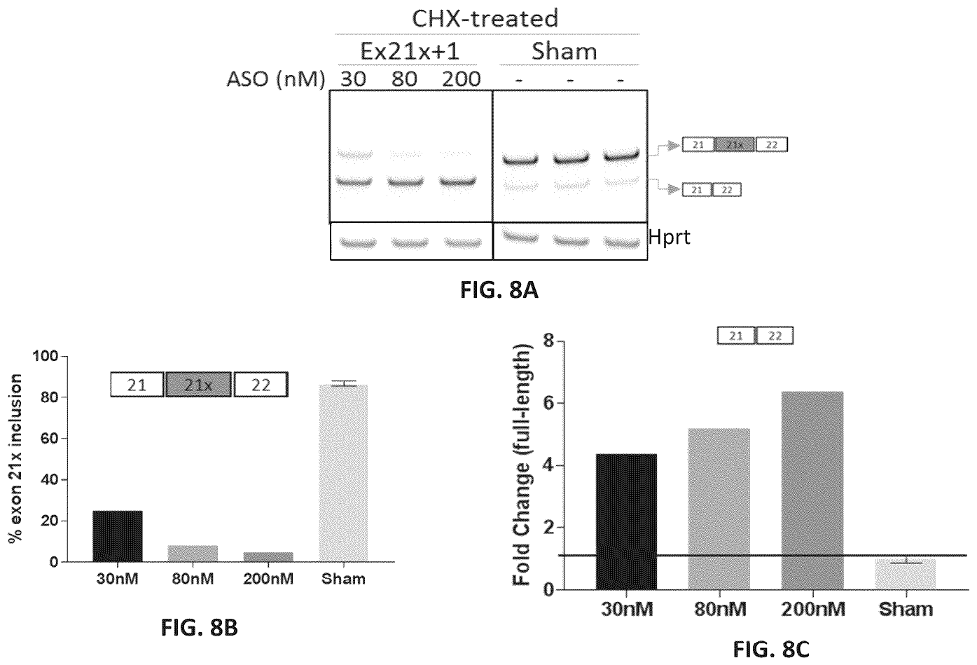

In FIG. 8A, a representative PAGE shows SYBR-safe-stained RT-PCR products of mouse Scn1a mock-treated (Sham, RNAiMAX alone), or treated with Ex21×+1 2′-MOE ASO targeting the exon 21× (mouse nomenclature, corresponds to human exon 20×), at 30 nM, 80 nM, and 200 nM concentrations in Neuro 2A (mouse neuroblastoma) cells by RNAiMAX transfection. Ex21×+1 (mouse nomenclature) and Ex20×+1 (human nomenclature) are identical. Two products corresponding to exon 21× inclusion (top band) and full-length (exon 21× exclusion, bottom band) were quantified and percent exon 21× inclusion is plotted in the bar graph (FIG. 8B). The full-length products were also normalized to HPRT internal control and fold-change relative to Sham is plotted in the bar graph (FIG. 8C). The black line indicates a ratio of 1 and no change with respect to Sham.

### Example 7: Intravitreal (IVT) Injection of Selected ASOs

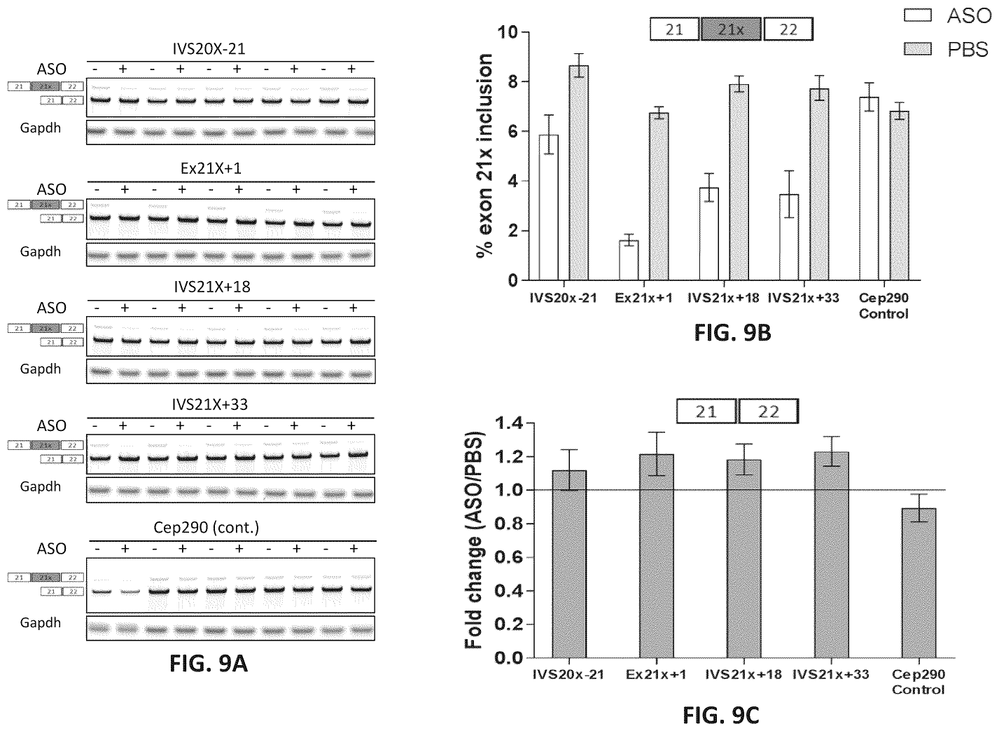

FIG. 9A shows PAGEs of SYBR-safe-stained RT-PCR products of mouse Scn1a from PBS-injected (1 μL) left eye (−) or IVS20×-21, Ex21×+1, IVS21×+18, IVS21×+33 or Cep290 (negative control ASO; Gerard et al, Mol. Ther. Nuc. Ac., 2015) 2′-MOE ASO-injected (1 μL) right eye (+) at 10 mM concentration. Ex21×+1, IVS21×+18, and IVS21×+33 (mouse nomenclature) and Ex20×+1, IVS20×+18, and IVS20×+33 (human nomenclature) are identical. Two products corresponding to exon 21× inclusion (top band) and full-length (exon 21× exclusion, bottom band) were quantified and percent exon 21× inclusion is plotted in FIG. 9B. White bars correspond to ASO-injected eyes and grey bars correspond to PBS-injected eyes, n=5 in each group. The full-length products were normalized to GAPDH internal control and fold-change of ASO-injected eye relative to PBS-injected eye is plotted in FIG. 9C. The black line indicates a ratio of 1 and no change with respect to PBS, n=5 in each group.

### Example 8: Intracerebroventricular (ICV) Injection of Selected ASOs

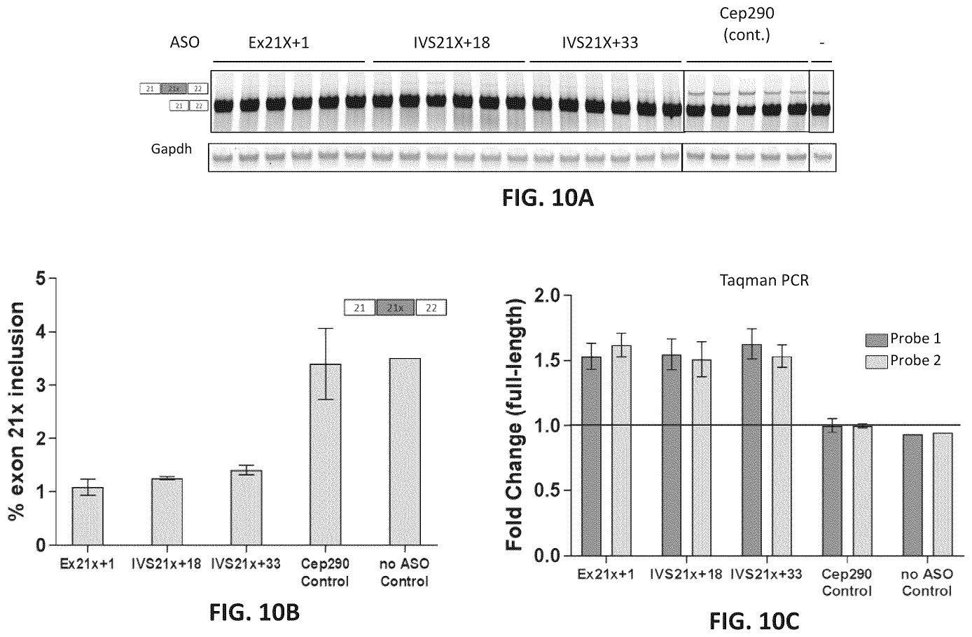

FIG. 10A shows PAGEs of SYBR-safe-stained RT-PCR products of mouse Scn1a from uninjected (−, no ASO control), or 300 μg of Cep290 (negative control ASO; Gerard et al, Mol. Ther. Nuc. Ac., 2015), Ex21×+1, IVS21×+18, IVS21×+33 2′-MOE ASO-injected brains. Ex21×+1, IVS21×+18, and IVS21×+33 (mouse nomenclature) and Ex20×+1, IVS20×+18, and IVS20×+33 (human nomenclature) are identical. Two products corresponding to exon 21× inclusion (top band) and full-length (exon 21× exclusion, bottom band) were quantified and percent exon 21× inclusion was plotted in the bar graph in FIG. 10B, n=6 (each targeting ASO), n=5 (Cep290 ASO), n=1 (uninjected, no ASO control). Tagman PCR was performed using two different probes spanning exons 21 and 22 junction and the products were normalized to GAPDH internal control and fold-change of ASO-injected relative to Cep290-injected brains was plotted in the bar graph in FIG. 10C. The black line indicates a ratio of 1 and no change with respect to Cep290, n=6 (each targeting ASO), n=5 (Cep290 ASO), n=1 (uninjected, no ASO control).

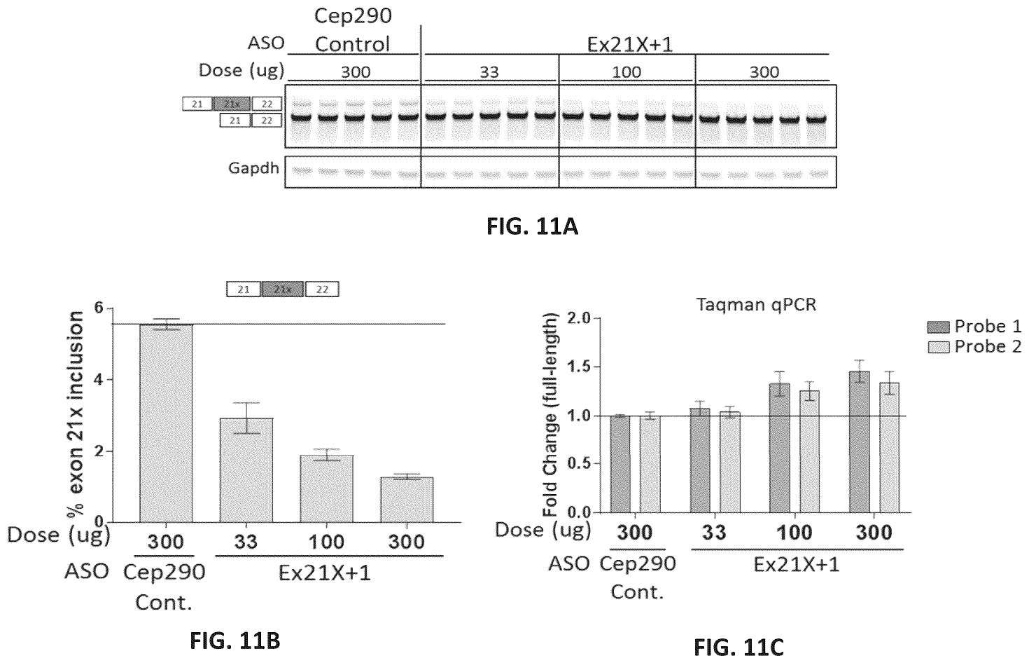

FIGS. 11A-C depict exemplary dose-dependent response from ICV injection of selected ASOs in C57BL6J mice (male, 3 months old). FIG. 11A shows PAGE gels of SYBR-safe-stained RT-PCR products of mouse Scn1a from 300 ug of Cep290 (negative control ASO; Gerard et al, Mol. Ther. Nuc. Ac., 2015), or 33 ug, 100 ug, and 300 ug of Ex21×+1 2′-MOE ASO-injected brains. Ex21×+1 (mouse nomenclature) and Ex20×+1, (human nomenclature) are identical. Two products corresponding to exon 21× inclusion (top band) and full-length (exon 21× exclusion, bottom band) were quantified. FIG. 11B depicts a graph plotting the percent exon 21× inclusion from the data in FIG. 11A, n=5 (each group). FIG. 11C depicts a graph from results of a Tagman qPCR assay performed using two different probes spanning exons 21 and 22 junction. The products were normalized to Gapdh internal control and fold-change of ASO-injected relative to Cep290-injected brains is plotted. The black line indicates a ratio of 1 and no change with respect to Cep290, n=5 (each group).

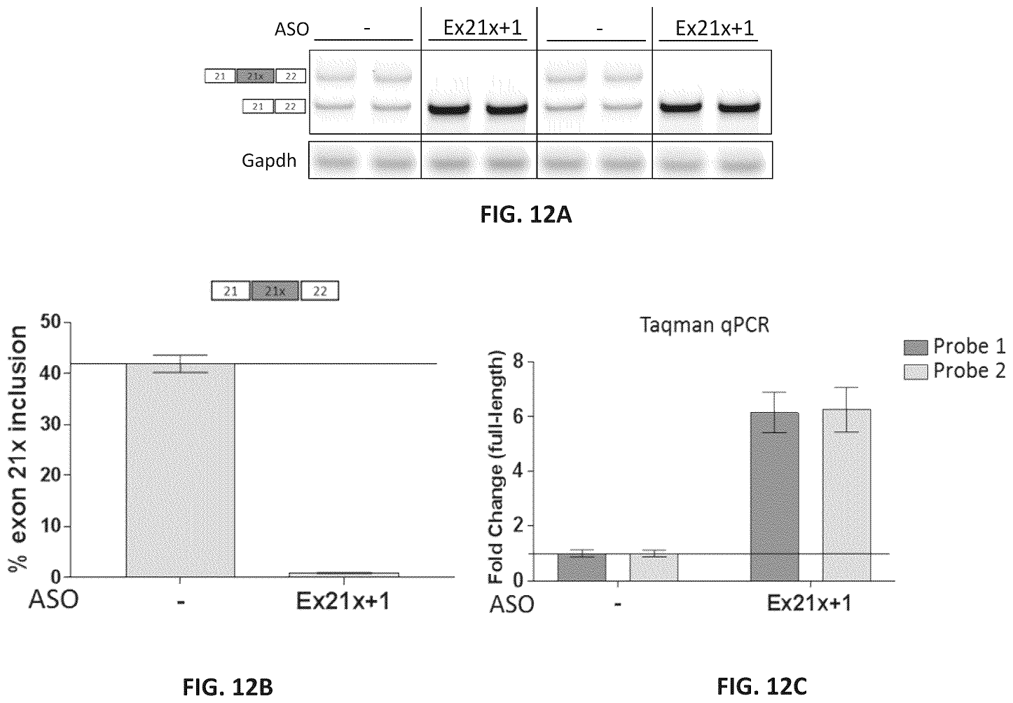

FIGS. 12A-C depict exemplary results from ICV injection of a selected ASO in C57BL6J mice (postnatal day 2). FIG. 12A shows PAGE gels of SYBR-safe-stained RT-PCR products of mouse Scn1a from uninjected (−, no ASO control), or 20 μg Ex21×+1 2′-MOE ASO-injected brains are shown. Two products corresponding to exon 21× inclusion (top band) and full-length (exon 21× exclusion, bottom band) were quantified. Ex21×+1 (mouse nomenclature) and Ex20×+1 (human nomenclature) are identical. FIG. 12B depicts a graph plotting the percent exon 21× inclusion from the data in FIG. 12A, n=4 (each group). FIG. 12C depicts a graph from results of a Tagman qPCR assay performed using two different probes spanning exons 21 and 22 junction. The products were normalized to Gapdh internal control and fold-change of ASO-injected relative to no-ASO-control brains is plotted. The black line indicates a ratio of 1 and no change with respect to no-ASO control, n=4 (each group).

### Example 9: Targeted Augmentation of Nuclear Gene Output for the Treatment of Dravet Syndrome

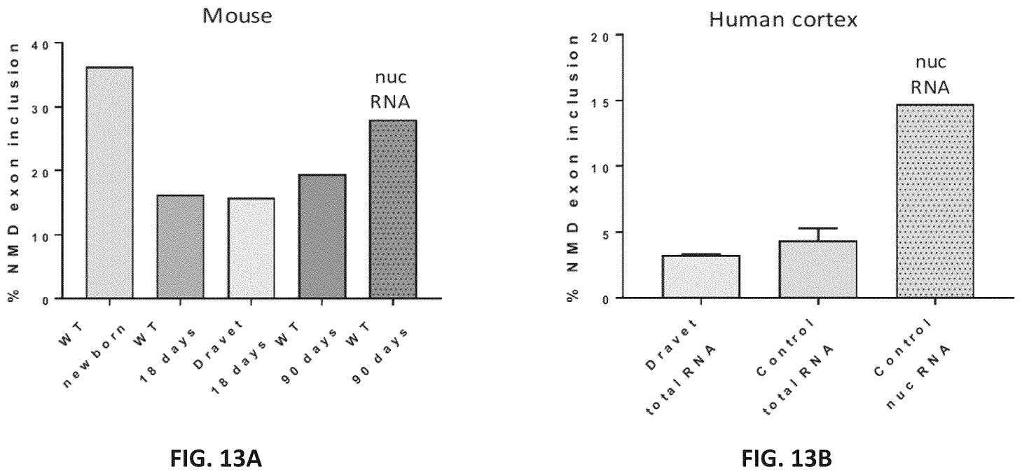

Dravet syndrome (DS) is a devastating childhood genetic disease characterized by severe seizures, cognitive & motor impairments and death. The primary cause of DS is decreased expression of the sodium voltage-gated channel type 1 alpha subunit (Nav1.1). SCN1A non-productive splicing event is conserved between human and mouse. FIG. 13A depicts a graph plotting the percent exon 21× inclusion in the indicated mouse CNS samples. FIG. 13B depicts a graph plotting the percent exon 20× inclusion in the indicated human CNS samples. In this study, an antisense oligonucleotides (ASO) therapy was utilized to increase productive Scn1a mRNA and consequently restore levels of Nav1.1 protein.

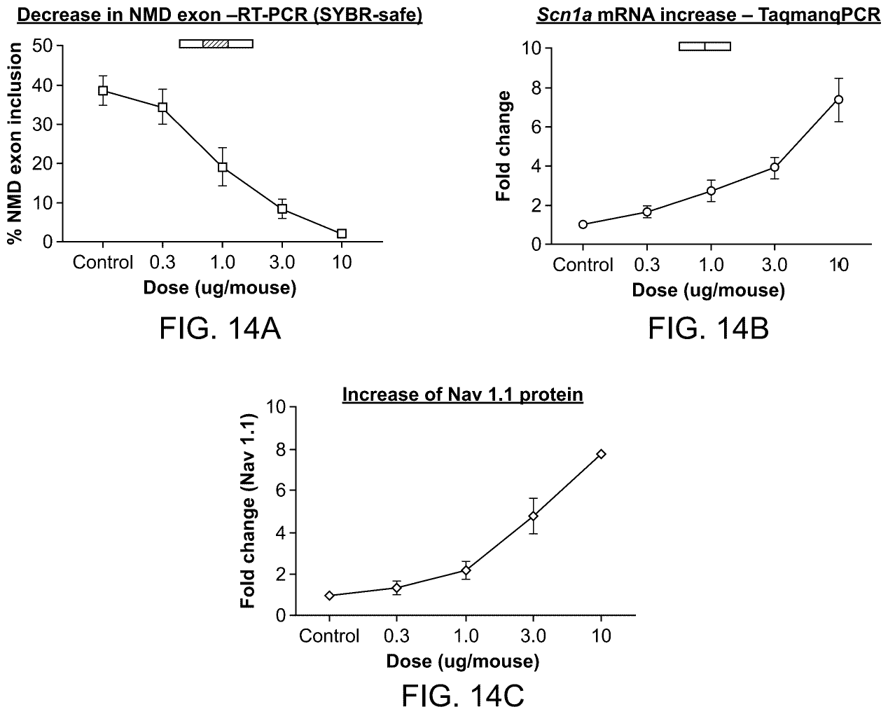

FIG. 14A depicts a graph plotting the percent decrease in exon 21× inclusion at the indicated doses (n=3-6 per group). FIG. 14B depicts a graph plotting the percent increase in Scn1a mRNA at the indicated doses (n=3-6 per group). FIG. 14C depicts a graph plotting the percent increase in Nav 1.1 protein levels at the indicated doses (n=2 per group).

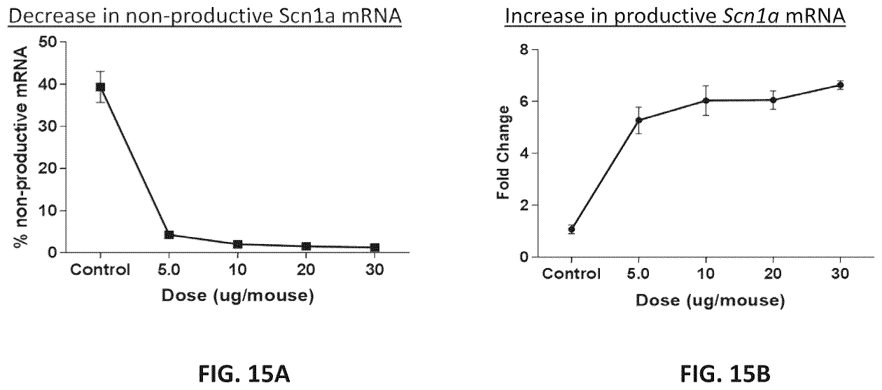

FIG. 15A depicts a graph plotting the percent decrease in exon 21× inclusion at the indicated doses (n=4 per group). FIG. 15B depicts a graph plotting the percent increase in Scn1a mRNA at the indicated doses (n=4 per group).

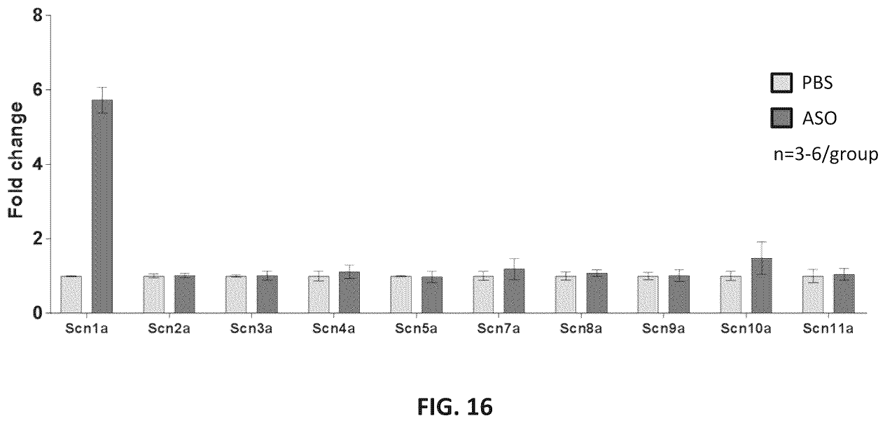

FIG. 16 depicts a selected Scn1a targeting ASO administered at a 10 ug dose via ICV injection in postnatal day 2 mice evaluated at day 5 post-injection by Tagman qPCR of SCN1A, SCN2A, SCN3A, SCN4A, SCN5A, SCN7A, SCN8A, SCN9A, SCN10A, and SCN11A to assess target selectivity. Tagman-qPCR amplification results normalized to Gapdh, obtained using Ex20×+1 ASO, are plotted as fold change relative to PBS injected mice (n=3-6 per group).

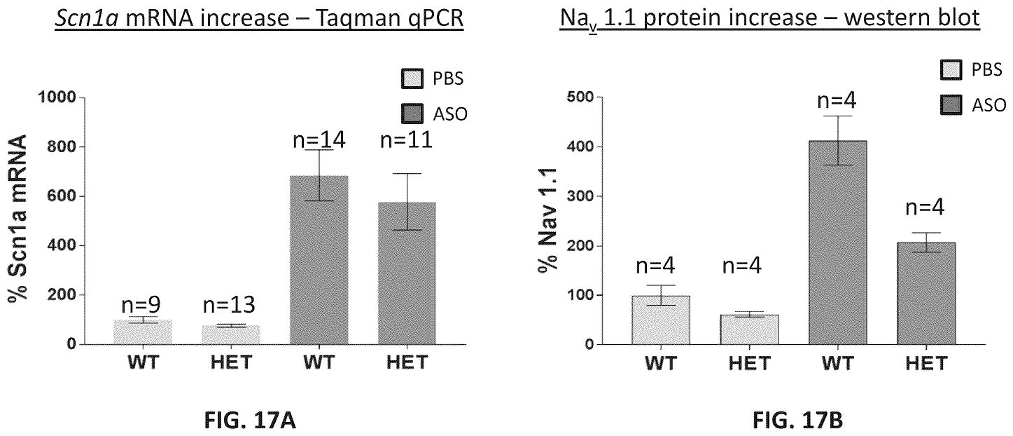

FIGS. 17A-B depict exemplary results from intracerebroventricular (ICV) injection at postnatal day 2 of a selected ASO at the indicated dose in wild type (WT) or heterozygous Dravet mice (HET) F1 mice from 129S-Scn1a^{tmiKea}×C57BL/6J crosses at 3 days post-injection (n=9-14 per group). FIG. 17A depicts a graph from results of a Tagman qPCR assay performed using a probe spanning exons 21 and 22. The products were normalized to Gapdh internal control and fold-change of ASO-injected relative to PBS-injected brains is plotted. FIG. 17B depicts a graph from results of a western blot performed using an anti-Nav1.1 antibody. The products were normalized to Ponceau-stained bands and fold-change of ASO-injected relative to PBS-injected brains is plotted.

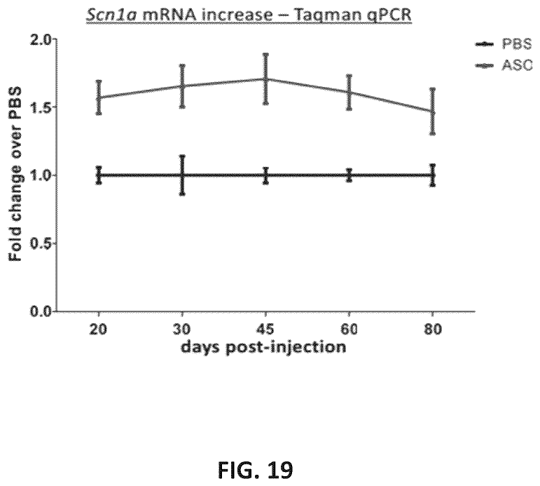

FIG. 19 is a graph plotting increase in Scn1a mRNA level in coronal brain slices of mice over the time post ICV injection of a SCN1A targeting ASO. As depicted, increase in Scn1a mRNA level, as quantified by Tagman qPCR, was maintained for at least 80 days post-injection (n=3-9 per group).

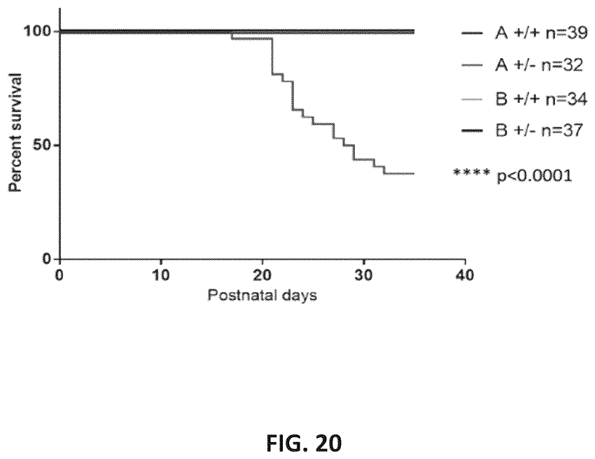

FIG. 20 is an exemplary survival curve demonstrating 100% survival benefit provided by a SCN1A targeting ASO in Dravet mouse model. WT and heterozygous Dravet mice (+/−), F1 offspring from 129S-scn1a^{tmiKea}×C57BL/6J crosses, received a single dose ICV injection of 20 μg PBS or ASO blindly (treatment marked as A or B) on postnatal day 2, and their survival was monitored. As depicted, mice in A +/− group (Dravet mice receiving PBS treatment) started to die from about postnatal day 16, whereas all mice of other three groups, including B +/−(Drave mice receiving ASO treatment) group, survived at least 35 days postnatal (n=32-39 per group).

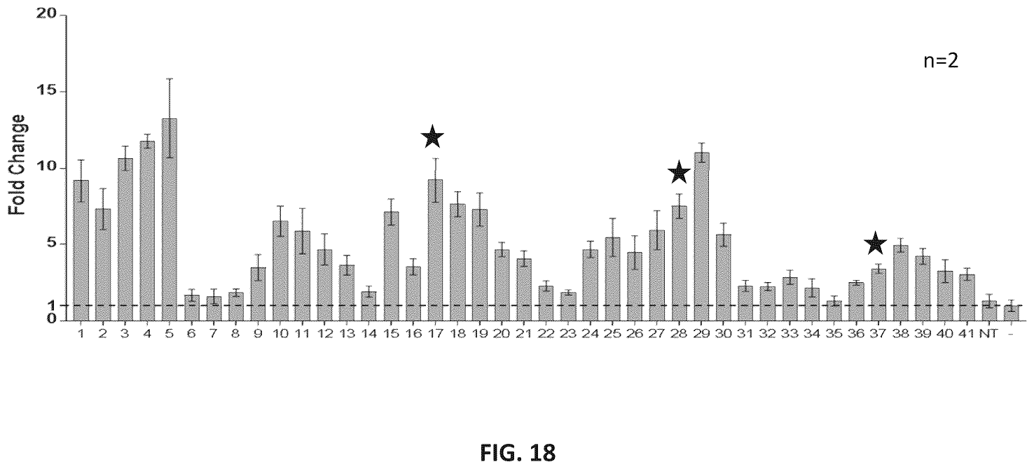

FIG. 18 depicts exemplary results of a SCN1A exon 20× region ASO microwalk in RenCells via free uptake. ASOs were designed to cover regions around three previously identified targeting ASOs in FIG. 6 (marked by stars) by shifting 1 nucleotides at a time (6-41) or by decreasing the length of ASO 17 (1-5). The graph depicts the percent exon 20× inclusion as measured by SYBR-green qPCR. The black line indicates no change with respect to no ASO (−).

Sequences of ASOs are summarized in Table 7a and Table 7b.

TABLE 7a
Sequences of ASOs targeting human SCN1A

| ASO ID | ASOs targeting Sequence 5′-3′ | SEQ ID NO: |
|---|---|---|
| 1 | TTGGAGCAAGATTATC | 304 |
| 2 | GTTGGAGCAAGATTATC | 305 |
| 3 | GTTGGAGCAAGATTAT | 306 |
| 4 | AGTTGGAGCAAGATTAT | 307 |
| 5 | AGTTGGAGCAAGATTA | 308 |
| 6 | GATTATCCTATACAAAAT | 309 |
| 7 | AGATTATCCTATACAAAA | 310 |
| 8 | AAGATTATCCTATACAAA | 311 |
| 9 | CAAGATTATCCTATACAA | 312 |
| 10 | GCAAGATTATCCTATACA | 313 |
| 11 | AGCAAGATTATCCTATAC | 314 |
| 12 | GAGCAAGATTATCCTATA | 315 |
| 13 | GGAGCAAGATTATCCTAT | 316 |
| 14 | TGGAGCAAGATTATCCTA | 317 |
| 15 | GTTGGAGCAAGATTATCC | 318 |
| 16 | TTGGAGCAAGATTATCCT | 319 |
| 18 | AAGTTGGAGCAAGATTAT | 320 |
| 19 | CAAGTTGGAGCAAGATTA | 321 |
| 20 | CCAAGTTGGAGCAAGATT | 322 |
| 21 | TCCAAGTTGGAGCAAGAT | 323 |
| 22 | AGTACCCATAATAAAGGG | 324 |
| 23 | AATACAGTACCCATAATA | 325 |
| 24 | ATTAAAGGTAGCAAAAGG | 326 |
| 25 | GATTAAAGGTAGCAAAAG | 327 |
| 26 | GGATTAAAGGTAGCAAAA | 328 |
| 27 | AGGATTAAAGGTAGCAAA | 329 |
| 29 | CAAGGATTAAAGGTAGCA | 330 |
| 30 | GCAAGGATTAAAGGTAGC | 331 |
| 31 | TGCAAGGATTAAAGGTAG | 332 |
| 32 | GTGCAAGGATTAAAGGTA | 333 |
| 33 | AGTCACAGTGCAAGGATT | 334 |
| 34 | AAGTCACAGTGCAAGGAT | 335 |
| 35 | TAAGTCACAGTGCAAGGA | 336 |
| 36 | ATAAGTCACAGTGCAAGG | 337 |
| 38 | ACATAAGTCACAGTGCAA | 338 |
| 39 | CACATAAGTCACAGTGCA | 339 |
| 40 | ACACATAAGTCACAGTGC | 340 |
| 41 | TACACATAAGTCACAGTG | 341 |

TABLE 7b
Sequences of ASOs targeting human SCN1A

| ASO ID | Sequence 5'-3' | SEQ ID NO: |
|---|---|---|
| 1_U | UUGGAGCAAGAUUAUC | 342 |
| 2_U | GUUGGAGCAAGAUUAUC | 343 |
| 3_U | GUUGGAGCAAGAUUAU | 344 |
| 4_U | AGUUGGAGCAAGAUUAU | 345 |
| 5_U | AGUUGGAGCAAGAUUA | 346 |
| 6_U | GAUUAUCCUAUACAAAAU | 347 |
| 7_U | AGAUUAUCCUAUACAAAA | 348 |
| 8_U | AAGAUUAUCCUAUACAAA | 349 |
| 9_U | CAAGAUUAUCCUAUACAA | 350 |
| 10_U | GCAAGAUUAUCCUAUACA | 351 |
| 11_U | AGCAAGAUUAUCCUAUAC | 352 |
| 12_U | GAGCAAGAUUAUCCUAUA | 353 |
| 13_U | GGAGCAAGAUUAUCCUAU | 354 |
| 14_U | UGGAGCAAGAUUAUCCUA | 355 |
| 15_U | GUUGGAGCAAGAUUAUCC | 356 |
| 16_U | UUGGAGCAAGAUUAUCCU | 357 |
| 18_U | AAGUUGGAGCAAGAUUAU | 358 |
| 19_U | CAAGUUGGAGCAAGAUUA | 359 |
| 20_U | CCAAGUUGGAGCAAGAUU | 360 |
| 21_U | UCCAAGUUGGAGCAAGAU | 361 |
| 22_U | AGUACCCAUAAUAAAGGG | 362 |
| 23_U | AAUACAGUACCCAUAAUA | 363 |
| 24_U | AUUAAAGGUAGCAAAAGG | 364 |
| 25_U | GAUUAAAGGUAGCAAAAG | 365 |
| 26_U | GGAUUAAAGGUAGCAAAA | 366 |
| 27_U | AGGAUUAAAGGUAGCAAA | 367 |
| 29_U | CAAGGAUUAAAGGUAGCA | 368 |
| 30_U | GCAAGGAUUAAAGGUAGC | 369 |
| 31_U | UGCAAGGAUUAAAGGUAG | 370 |
| 32_U | GUGCAAGGAUUAAAGGUA | 371 |
| 33_U | AGUCACAGUGCAAGGAUU | 372 |
| 34_U | AAGUCACAGUGCAAGGAU | 373 |
| 35_U | UAAGUCACAGUGCAAGGA | 374 |
| 36_U | AUAAGUCACAGUGCAAGG | 375 |
| 38_U | ACAUAAGUCACAGUGCAA | 376 |
| 39_U | CACAUAAGUCACAGUGCA | 377 |
| 40_U | ACACAUAAGUCACAGUGC | 378 |
| 41_U | UACACAUAAGUCACAGUG | 379 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-2. (canceled)

3. A modified antisense oligomer, wherein the modified antisense oligomer comprises at least one modified nucleotide, wherein the modified nucleotide comprises a modification of the backbone linkage, a modification of a sugar moiety, or a combination thereof, and wherein the modified antisense oligomer comprises a sequence that is complementary to a target sequence of a pre-mRNA in a cell that contains a non-sense mediated RNA decay inducing exon (NMD exon) and that encodes Nav1.1 protein, whereby hybridization of the modified antisense oligomer to the target sequence modulates splicing of the NMD exon from the pre-mRNA in the cell that contains the NMD exon and that encodes Nav1.1.

4. The modified antisense oligomer of claim 3, wherein the modified antisense oligomer increases a level of processed mRNA encoding the Nav1.1 protein in the cell having the pre-mRNA that contains the NMD exon and that encodes Nav1.1 when the modified antisense oligomer is introduced into the cell.

5. The modified antisense oligomer of claim 3, wherein the modified antisense oligomer increases a level of the Nav1.1 protein in the cell having the pre-mRNA that contains the NMD exon and that encodes Nav1.1 when the modified antisense oligomer is introduced into the cell.

6. The modified antisense oligomer of claim 3, wherein the target sequence:
(a) is within an intron sequence flanking the NMD exon;
(b) comprises at least one nucleotide of the NMD exon; or
(c) is within the NMD exon.

7. The modified antisense oligomer of claim 3, wherein the pre-mRNA that contains the NMD exon and that encodes Nav1.1 comprises a sequence with at least about 95% sequence identity to any one of SEQ ID NOs: 2 or 7-10.

8. The modified antisense oligomer of claim 3, wherein the target sequence of the pre-mRNA that contains the NMD exon and that encodes Nav1.1 comprises a sequence with at least about 95% sequence identity to a region comprising at least 8 contiguous nucleic acids of SEQ ID NO: 2 or 7-10.

9. The modified antisense oligomer of claim 3, wherein the modified nucleotide comprises hypoxanthine, xanthine, 7-methylguanine, 5, 6-dihydrouracil, 5-methylcytosine, or a 5-hydroxymethylcytosine.

10. The modified antisense oligomer of claim 3, wherein the modification of the backbone linkage comprises a phosphorothioate, phosphorodithioate, phosphoroselenoate, phosphorodiselenoate, phosphoroanilothioate, phosphoranilidate, or phosphoramidate linkage.

11. The modified antisense oligomer of claim 3, wherein the modification of the backbone linkage comprises a phosphorothioate linkage.

12. The modified antisense oligomer of claim 3, wherein the modification of the backbone linkage comprises a phosphoramidate linkage.

13. The modified antisense oligomer of claim 3, wherein the modification of the backbone linkage comprises one or more peptide bonds.

14. The modified antisense oligomer of claim 3, wherein the modification of the sugar moiety is selected from the group consisting of a 2′-O-methyl, a 2′-O-aminoethyl, a 2′-Fluoro, and a 2′-O-methoxyethyl modification.

15. The modified antisense oligomer of claim 3, wherein the modification of the sugar moiety is a morpholine ring.

16. The modified antisense oligomer of claim 15, wherein the morpholine ring is a phosphoramidite morpholino (PMO) or a locked nucleic acid (LNA).

17. The modified antisense oligomer of claim 3, wherein the modified antisense oligomer is from 8 to 50 nucleobases in length.

18. The modified antisense oligomer of claim 3, wherein each modified nucleotide of the antisense oligomer comprises one or more identical modifications.

19. The modified antisense oligomer of claim 3, wherein at least two of the modified nucleotides of the antisense oligomer comprise different modifications.

20. The modified antisense oligomer of claim 3, wherein the modified antisense oligomer comprises a 2′-O-methoxyethyl moiety.

21. The modified antisense oligomer of claim 3, wherein each nucleotide of the modified antisense oligomer comprises a 2′-O-methoxyethyl moiety.

22. The modified antisense oligomer of claim 3 wherein the modified antisense oligomer comprises a sequence that has at least about 80% sequence identity to any one of SEQ ID NOs: 21-61, 64-67, 210-250, 253-256, or 304-379.

23. The modified antisense oligomer of claim 3, wherein the modified antisense oligomer comprises a sequence that has at least about 90% sequence identity to any one of SEQ ID NOs: 21-61, 64-67, 210-250, 253-256, or 304-379.

24. The modified antisense oligomer of claim 3, wherein the modified antisense oligomer is a sequence according to any one of SEQ ID NOs: 21-61, 64-67, 210-250, 253-256, or 304-379.
